SEQUENCE LISTING (226 sequences)

SEQ ID NO: 1 - protein, 5 aa, synthetic construct
GYGMS

SEQ ID NO: 2 - protein, 17 aa, synthetic construct
YISGYSYYTYYADSVKG

SEQ ID NO: 3 - protein, 8 aa, synthetic construct
YDGNGFDY

SEQ ID NO: 4 - protein, 13 aa, synthetic construct
SGSSSNIGSNYVY

SEQ ID NO: 5 - protein, 7 aa, synthetic construct
RNNQRPS

SEQ ID NO: 6 - protein, 8 aa, synthetic construct
AADSSDDS

SEQ ID NO: 7 - protein, 117 aa, synthetic construct
EVQLLESGGGLVQPGGSLRLSCAASGFTFSGYGMSWVRQAPGKGLEWVSYISGYSYYTYYADSVKGRFTFSRDNSKNTLYLQMNSLRAEDTAVYYCARYDGNGFDYWGQGTLVTVSS

SEQ ID NO: 8 - protein, 110 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAADSSDDSYVFGGGTKLTVLG

SEQ ID NO: 9 - protein, 242 aa, synthetic construct
EVQLLESGGGLVQPGGSLRLSCAASGFTFSGYGMSWVRQAPGKGLEWVSYISGYSYYTYYADSVKGRFTFSRDNSKNTLYLQMNSLRAEDTAVYYCARYDGNGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADSSDDSYVFGGGTKLTVLG

SEQ ID NO: 10 - protein, 470 aa, synthetic construct
EVQLLESGGGLVQPGGSLRLSCAASGFTFSGYGMSWVRQAPGKGLEWVSYISGYSYYTYYADSVKGRFTFSRDNSKNTLYLQMNSLRAEDTAVYYCARYDGNGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADSSDDSYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 15 bp, synthetic construct
  source 1..15: mol_type = other DNA
ggttatggtatgagc

SEQ ID NO: 12 - DNA, 51 bp, synthetic construct
  source 1..51: mol_type = other DNA
tacatctctggttactcttactacacgtattacgctgattctgtaaaaggt

SEQ ID NO: 13 - DNA, 24 bp, synthetic construct
  source 1..24: mol_type = other DNA
tacgacggtaacggtttcgactac

SEQ ID NO: 14 - DNA, 39 bp, synthetic construct
  source 1..39: mol_type = other DNA
agtggctcttcatctaatattggctctaattatgtctac

SEQ ID NO: 15 - DNA, 21 bp, synthetic construct
  source 1..21: mol_type = other DNA
agaaataaccagcggccaagc

SEQ ID NO: 16 - DNA, 24 bp, synthetic construct
  source 1..24: mol_type = other DNA
gctgctgactcttctgacgactct

SEQ ID NO: 17 - DNA, 351 bp, synthetic construct
  source 1..351: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctctggttactcttactacacgtattacgctgattctgtaaaaggtcggttcaccttctccagagacaattccaagaacacgctgtatctgcaaatgaacagcctgagagccgaggacacggccgtgtattactgtgcgcgttacgacggtaacggtttcgactactggggccagggtacactggtcaccgtgagctca

SEQ ID NO: 18 - DNA, 330 bp, synthetic construct
  source 1..330: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactcttctgacgactcttatgtcttcggcggaggcaccaagctgacggtcctaggt

SEQ ID NO: 19 - DNA, 726 bp, synthetic construct
  source 1..726: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctctggttactcttactacacgtattacgctgattctgtaaaaggtcggttcaccttctccagagacaattccaagaacacgctgtatctgcaaatgaacagcctgagagccgaggacacggccgtgtattactgtgcgcgttacgacggtaacggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactcttctgacgactcttatgtcttcggcggaggcaccaagctgacggtcctaggt

SEQ ID NO: 20 - DNA, 1410 bp, synthetic construct
  source 1..1410: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctctggttactcttactacacgtattacgctgattctgtaaaaggtcggttcaccttctccagagacaattccaagaacacgctgtatctgcaaatgaacagcctgagagccgaggacacggccgtgtattactgtgcgcgttacgacggtaacggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactcttctgacgactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 21 - protein, 5 aa, synthetic construct
GYGMS

SEQ ID NO: 22 - protein, 17 aa, synthetic construct
YISSGSYYTYYADSVKG

SEQ ID NO: 23 - protein, 13 aa, synthetic construct
YRGDNDYYGYFDY

SEQ ID NO: 24 - protein, 13 aa, synthetic construct
SCSSSNIGNNAVS

SEQ ID NO: 25 - protein, 7 aa, synthetic construct
RNNQRPS

SEQ ID NO: 26 - protein, 7 aa, synthetic construct
AADYGSD

SEQ ID NO: 27 - protein, 122 aa, synthetic construct
EVQLLESGGGLVQPGGSLRLSCAASGFTFSGYGMSWVRQAPGKGLEWVSYISSGSYYTYYADSVKGRFTISRDNSKNTLYLQMNSLRAEDTAVYYCARYRGDNDYYGYFDYWGQGTLVTVSS

SEQ ID NO: 28 - protein, 109 aa, synthetic construct
QSVLTQPPSASGTPGQRVTISCSCSSSNIGNNAVSWYQQLPGTAPKLLIYRNNQRPSGVPDRFSGSKSGTSASLAISGLRSEDEADYYCAADYGSDYVFGGGTKLTVLG

SEQ ID NO: 29 - protein, 246 aa, synthetic construct
EVQLLESGGGLVQPGGSLRLSCAASGFTFSGYGMSWVRQAPGKGLEWVSYISSGSYYTYYADSVKGRFTISRDNSKNTLYLQMNSLRAEDTAVYYCARYRGDNDYYGYFDYWGQGTLVTVSSGGGGSGGGGSGGGGSQSVLTQPPSASGTPGQRVTISCSCSSSNIGNNAVSWYQQLPGTAPKLLIYRNNQRPSGVPDRFSGSKSGTSASLAISGLRSEDEADYYCAADYGSDYVFGGGTKLTVLG

SEQ ID NO: 30 - protein, 474 aa, synthetic construct
EVQLLESGGGLVQPGGSLRLSCAASGFTFSGYGMSWVRQAPGKGLEWVSYISSGSYYTYYADSVKGRFTISRDNSKNTLYLQMNSLRAEDTAVYYCARYRGDNDYYGYFDYWGQGTLVTVSSGGGGSGGGGSGGGGSQSVLTQPPSASGTPGQRVTISCSCSSSNIGNNAVSWYQQLPGTAPKLLIYRNNQRPSGVPDRFSGSKSGTSASLAISGLRSEDEADYYCAADYGSDYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 15 bp, synthetic construct
  source 1..15: mol_type = other DNA
ggttatggtatgagc

SEQ ID NO: 32 - DNA, 51 bp, synthetic construct
  source 1..51: mol_type = other DNA
tacatctcttctggttcttactacacgtattacgctgattctgtaaaaggt

SEQ ID NO: 33 - DNA, 39 bp, synthetic construct
  source 1..39: mol_type = other DNA
taccgtggtgacaacgattactacggttacttcgactac

SEQ ID NO: 34 - DNA, 39 bp, synthetic construct
  source 1..39: mol_type = other DNA
agttgctcttcatctaatattggcaataatgctgtctcc

SEQ ID NO: 35 - DNA, 21 bp, synthetic construct
  source 1..21: mol_type = other DNA
agaaataaccagcggccaagc

SEQ ID NO: 36 - DNA, 21 bp, synthetic construct
  source 1..21: mol_type = other DNA
gctgctgactacggttctgac

SEQ ID NO: 37 - DNA, 366 bp, synthetic construct
  source 1..366: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctcttctggttcttactacacgtattacgctgattctgtaaaaggtcggttcaccatctccagagacaattccaagaacacgctgtatctgcaaatgaacagcctgagagccgaggacacggccgtgtattactgtgcgcgttaccgtggtgacaacgattactacggttacttcgactactggggccagggtacactggtcaccgtgagctca

SEQ ID NO: 38 - DNA, 327 bp, synthetic construct
  source 1..327: mol_type = other DNA
cagtctgtgctgactcagccaccctcagcgtctgggacccccgggcagagggtcaccatctcttgtagttgctcttcatctaatattggcaataatgctgtctcctggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactacggttctgactatgtcttcggcggaggcaccaagctgacggtcctaggt

SEQ ID NO: 39 - DNA, 738 bp, synthetic construct
  source 1..738: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctcttctggttcttactacacgtattacgctgattctgtaaaaggtcggttcaccatctccagagacaattccaagaacacgctgtatctgcaaatgaacagcctgagagccgaggacacggccgtgtattactgtgcgcgttaccgtggtgacaacgattactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagttgctcttcatctaatattggcaataatgctgtctcctggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactacggttctgactatgtcttcggcggaggcaccaagctgacggtcctaggt

SEQ ID NO: 40 - DNA, 1422 bp, synthetic construct
  source 1..1422: mol_type = other DNA
gaggtgcagctgttggagtctgggggaggcttggtacagcctggggggtccctgagactctcctgtgcagcctctggattcacctttagcggttatggtatgagctgggtccgccaggctccagggaaggggctggagtgggtctcatacatctcttctggttcttactacacgtattacgctgattctgtaaaaggtcggttcaccatctccagagacaattccaagaacacgctgtatctgcaaatgaacagcctgagagccgaggacacggccgtgtattactgtgcgcgttaccgtggtgacaacgattactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagttgctcttcatctaatattggcaataatgctgtctcctggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgactacggttctgactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 41 - protein, 4 aa, synthetic construct
SYMS

SEQ ID NO: 42 - protein, 17 aa, synthetic construct
SIGSGYYSTYYADSVKG

SEQ ID NO: 43 - protein, 8 aa, synthetic construct
DYYGGFDY

SEQ ID NO: 44 - protein, 13 aa, synthetic construct
SGSSSNIGSNYVY

SEQ ID NO: 45 - protein, 7 aa, synthetic construct
RNNQRPS

SEQ ID NO: 46 - protein, 7 aa, synthetic construct
AAYDSYS

SEQ ID NO: 47 - protein, 116 aa, synthetic construct
EVQLLESGGGLVQPGGSLRLSCAASGFTFSSYMSWVRQAPGKGLEWVSSIGSGYYSTYYADSVKGRFTISRDNSKNTLYLQMNSLRAEDTAVYYCARDYYGGFDYWGQGTLVTVSS

SEQ ID NO: 48 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAYDSYSYVFGGGTKLTVLG

SEQ ID NO: 49 - protein, 240 aa, synthetic construct
EVQLLESGGGLVQPGGSLRLSCAASGFTFSSYMSWVRQAPGKGLEWVSSIGSGYYSTYYADSVKGRFTISRDNSKNTLYLQMNSLRAEDTAVYYCARDYYGGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYDSYSYVFGGGTKLTVLG

SEQ ID NO: 50 - protein, 468 aa, synthetic construct
EVQLLESGGGLVQPGGSLRLSCAASGFTFSSYMSWVRQAPGKGLEWVSSIGSGYYSTYYADSVKGRFTISRDNSKNTLYLQMNSLRAEDTAVYYCARDYYGGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYDSYSYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 12 bp, synthetic construct
  source 1..12: mol_type = other DNA
tcttacatgagc

SEQ ID NO: 52 - DNA, 51 bp, synthetic construct
  source 1..51: mol_type = other DNA
tctatcggttctggttactactctacgtattacgctgattctgtaaaaggt

SEQ ID NO: 53 - DNA, 24 bp, synthetic construct
  source 1..24: mol_type = other DNA
gactactacggtggtttcgactac

SEQ ID NO: 54 - DNA, 39 bp, synthetic construct; identical to SEQ ID NO: 14
  source 1..39: mol_type = other DNA

SEQ ID NO: 55 - DNA, 21 bp, synthetic construct
  source 1..21: mol_type = other DNA
agaaataaccagcggccaagc

SEQ ID NO: 56 - DNA, 21 bp, synthetic construct
  source 1..21: mol_type = other DNA
gctgcttacgactcttactct

SEQ ID NO: 57 - DNA, 348 bp, synthetic construct
  source 1..348: mol_type = other DNA
gaggtgcagctgttggagtctgggggaggcttggtacagcctggggggtccctgagactctcctgtgcagcctctggattcacctttagctcttacatgagctgggtccgccaggctccagggaaggggctggagtgggtctcatctatcggttctggttactactctacgtattacgctgattctgtaaaaggtcggttcaccatctccagagacaattccaagaacacgctgtatctgcaaatgaacagcctgagagccgaggacacggccgtgtattactgtgcgcgtgactactacggtggtttcgactactggggccagggtacactggtcaccgtgagctca

SEQ ID NO: 58 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacgactcttactcttatgtcttcggcggaggcaccaagctgacggtcctaggt

SEQ ID NO: 59 - DNA, 720 bp, synthetic construct
  source 1..720: mol_type = other DNA
gaggtgcagctgttggagtctgggggaggcttggtacagcctggggggtccctgagactctcctgtgcagcctctggattcacctttagctcttacatgagctgggtccgccaggctccagggaaggggctggagtgggtctcatctatcggttctggttactactctacgtattacgctgattctgtaaaaggtcggttcaccatctccagagacaattccaagaacacgctgtatctgcaaatgaacagcctgagagccgaggacacggccgtgtattactgtgcgcgtgactactacggtggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacgactcttactcttatgtcttcggcggaggcaccaagctgacggtcctaggt

SEQ ID NO: 60 - DNA, 1404 bp, synthetic construct
  source 1..1404: mol_type = other DNA
gaggtgcagctgttggagtctgggggaggcttggtacagcctggggggtccctgagactctcctgtgcagcctctggattcacctttagctcttacatgagctgggtccgccaggctccagggaaggggctggagtgggtctcatctatcggttctggttactactctacgtattacgctgattctgtaaaaggtcggttcaccatctccagagacaattccaagaacacgctgtatctgcaaatgaacagcctgagagccgaggacacggccgtgtattactgtgcgcgtgactactacggtggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacgactcttactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 61 - protein, 5 aa, synthetic construct
YYGMS

SEQ ID NO: 62 - protein, 17 aa, synthetic construct
GIGSYSSYTYYADSVKG

SEQ ID NO: 63 - protein, 11 aa, synthetic construct
YASSPDAYFDY

SEQ ID NO: 64 - protein, 13 aa, synthetic construct
SGSSSNIGSNYVY

SEQ ID NO: 65 - protein, 7 aa, synthetic construct
RNNQRPS

SEQ ID NO: 66 - protein, 7 aa, synthetic construct
AAYYNYN

SEQ ID NO: 67 - protein, 120 aa, synthetic construct
EVQLLESGGGLVQPGGSLRLSCAASGFTFSYYGMSWVRQAPGKGLEWVSGIGSYSSYTYYADSVKGRFTISRDNSKNTLYLQMNSLRAEDTAVYYCARYASSPDAYFDYWGQGTLVTVSS

SEQ ID NO: 68 - protein, 109 aa, synthetic construct
QSVLTQPPSASGTPGQRVTISCSGSSSNIGSNYVYWYQQLPGTAPKLLIYRNNQRPSGVPGRFSGSKSGTSASLAISGLRSEDEADYYCAAYYNYNYVFGGGTKLTVLG

SEQ ID NO: 69 - protein, 244 aa, synthetic construct
EVQLLESGGGLVQPGGSLRLSCAASGFTFSYYGMSWVRQAPGKGLEWVSGIGSYSSYTYYADSVKGRFTISRDNSKNTLYLQMNSLRAEDTAVYYCARYASSPDAYFDYWGQGTLVTVSSGGGGSGGGGSGGGGSQSVLTQPPSASGTPGQRVTISCSGSSSNIGSNYVYWYQQLPGTAPKLLIYRNNQRPSGVPGRFSGSKSGTSASLAISGLRSEDEADYYCAAYYNYNYVFGGGTKLTVLG

SEQ ID NO: 70 - protein, 472 aa, synthetic construct
EVQLLESGGGLVQPGGSLRLSCAASGFTFSYYGMSWVRQAPGKGLEWVSGIGSYSSYTYYADSVKGRFTISRDNSKNTLYLQMNSLRAEDTAVYYCARYASSPDAYFDYWGQGTLVTVSSGGGGSGGGGSGGGGSQSVLTQPPSASGTPGQRVTISCSGSSSNIGSNYVYWYQQLPGTAPKLLIYRNNQRPSGVPGRFSGSKSGTSASLAISGLRSEDEADYYCAAYYNYNYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 15 bp, synthetic construct
  source 1..15: mol_type = other DNA
tactatggtatgagc

SEQ ID NO: 72 - DNA, 51 bp, synthetic construct
  source 1..51: mol_type = other DNA
ggtatcggttcttactcttcttacacgtattacgctgattctgtaaaaggt

SEQ ID NO: 73 - DNA, 33 bp, synthetic construct
  source 1..33: mol_type = other DNA
tacgcttcttctccggacgcttacttcgactac

SEQ ID NO: 74 - DNA, 39 bp, synthetic construct; identical to SEQ ID NO: 14
  source 1..39: mol_type = other DNA

SEQ ID NO: 75 - DNA, 21 bp, synthetic construct
  source 1..21: mol_type = other DNA
agaaataaccagcggccaagc

SEQ ID NO: 76 - DNA, 21 bp, synthetic construct
  source 1..21: mol_type = other DNA
gctgcttactacaactacaac

SEQ ID NO: 77 - DNA, 360 bp, synthetic construct
  source 1..360: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcaggtatcggttcttactcttcttacacgtattacgctgattctgtaaaaggtcggttcaccatctccagagacaattccaagaacacgctgtatctgcaaatgaacagcctgagagccgaggacacggccgtgtattactgtgcgcgttacgcttcttctccggacgcttacttcgactactggggccagggtacactggtcaccgtgagctca

SEQ ID NO: 78 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctggccgattctctggctccaagtctggcacctcagcctccctggccatcagtgggctccggtccgaggatgaggccgattattactgtgctgcttactacaactacaactatgtcttcggcggaggcaccaagctgacggtcctaggt

SEQ ID NO: 79 - DNA, 732 bp, synthetic construct
  source 1..732: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcaggtatcggttcttactcttcttacacgtattacgctgattctgtaaaaggtcggttcaccatctccagagacaattccaagaacacgctgtatctgcaaatgaacagcctgagagccgaggacacggccgtgtattactgtgcgcgttacgcttcttctccggacgcttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctggccgattctctggctccaagtctggcacctcagcctccctggccatcagtgggctccggtccgaggatgaggccgattattactgtgctgcttactacaactacaactatgtcttcggcggaggcaccaagctgacggtcctaggt

SEQ ID NO: 80 - DNA, 1416 bp, synthetic construct
  source 1..1416: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcaggtatcggttcttactcttcttacacgtattacgctgattctgtaaaaggtcggttcaccatctccagagacaattccaagaacacgctgtatctgcaaatgaacagcctgagagccgaggacacggccgtgtattactgtgcgcgttacgcttcttctccggacgcttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctggccgattctctggctccaagtctggcacctcagcctccctggccatcagtgggctccggtccgaggatgaggccgattattactgtgctgcttactacaactacaactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 81 - protein, 5 aa, synthetic construct
SYGMS

SEQ ID NO: 82 - protein, 17 aa, synthetic construct
YISGGSYYTYYADSVKG

SEQ ID NO: 83 - protein, 12 aa, synthetic construct
YGYGYYDGSFDY

SEQ ID NO: 84 - protein, 13 aa, synthetic construct
SGSSSNIGSNYVY

SEQ ID NO: 85 - protein, 7 aa, synthetic construct
RNNQRPS

SEQ ID NO: 86 - protein, 7 aa, synthetic construct
AADGPYN

SEQ ID NO: 87 - protein, 121 aa, synthetic construct
EVQLLESGGGLVQPGGSLRLSCAASGFTFSSYGMSWVRQAPGKGLEWVSYISGGSYYTYYADSVKGRFTISRDNSKNTLYLQMNSLRAEDTAVYYCARYGYGYYDGSFDYWGQGTLVTVSS

SEQ ID NO: 88 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAADGPYNYVFGGGTKLTVLG

SEQ ID NO: 89 - protein, 245 aa, synthetic construct
EVQLLESGGGLVQPGGSLRLSCAASGFTFSSYGMSWVRQAPGKGLEWVSYISGGSYYTYYADSVKGRFTISRDNSKNTLYLQMNSLRAEDTAVYYCARYGYGYYDGSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADGPYNYVFGGGTKLTVLG

SEQ ID NO: 90 - protein, 473 aa, synthetic construct
EVQLLESGGGLVQPGGSLRLSCAASGFTFSSYGMSWVRQAPGKGLEWVSYISGGSYYTYYADSVKGRFTISRDNSKNTLYLQMNSLRAEDTAVYYCARYGYGYYDGSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADGPYNYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 15 bp, synthetic construct
  source 1..15: mol_type = other DNA
tcttatggtatgagc

SEQ ID NO: 92 - DNA, 51 bp, synthetic construct
  source 1..51: mol_type = other DNA
tacatctctggtggttcttactacacgtattacgctgattctgtaaaaggt

SEQ ID NO: 93 - DNA, 36 bp, synthetic construct
  source 1..36: mol_type = other DNA
tacggttacggttactacgacggttctttcgactac

SEQ ID NO: 94 - DNA, 39 bp, synthetic construct; identical to SEQ ID NO: 14
  source 1..39: mol_type = other DNA

SEQ ID NO: 95 - DNA, 21 bp, synthetic construct
  source 1..21: mol_type = other DNA
agaaataaccagcggccaagc

SEQ ID NO: 96 - DNA, 21 bp, synthetic construct
  source 1..21: mol_type = other DNA
gctgctgacggtccgtacaac

SEQ ID NO: 97 - DNA, 363 bp, synthetic construct
  source 1..363: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatacatctctggtggttcttactacacgtattacgctgattctgtaaaaggtcggttcaccatctccagagacaattccaagaacacgctgtatctgcaaatgaacagcctgagagccgaggacacggccgtgtattactgtgcgcgttacggttacggttactacgacggttctttcgactactggggccagggtacactggtcaccgtgagctca

SEQ ID NO: 98 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacggtccgtacaactatgtcttcggcggaggcaccaagctgacggtcctaggt

SEQ ID NO: 99 - DNA, 735 bp, synthetic construct
  source 1..735: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatacatctctggtggttcttactacacgtattacgctgattctgtaaaaggtcggttcaccatctccagagacaattccaagaacacgctgtatctgcaaatgaacagcctgagagccgaggacacggccgtgtattactgtgcgcgttacggttacggttactacgacggttctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacggtccgtacaactatgtcttcggcggaggcaccaagctgacggtcctaggt

SEQ ID NO: 100 - DNA, 1419 bp, synthetic construct
  source 1..1419: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatacatctctggtggttcttactacacgtattacgctgattctgtaaaaggtcggttcaccatctccagagacaattccaagaacacgctgtatctgcaaatgaacagcctgagagccgaggacacggccgtgtattactgtgcgcgttacggttacggttactacgacggttctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacggtccgtacaactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 101 - protein, 5 aa, synthetic construct
SYSMS

SEQ ID NO: 102 - protein, 16 aa, synthetic construct
GIGYPYYTYYADSVKG

SEQ ID NO: 103 - protein, 9 aa, synthetic construct
YYYDYGFDY

SEQ ID NO: 104 - protein, 13 aa, synthetic construct
SGSSSNIGSNYVY

SEQ ID NO: 105 - protein, 7 aa, synthetic construct
RNNQRPS

SEQ ID NO: 106 - protein, 7 aa, synthetic construct
AAYRSYD

SEQ ID NO: 107 - protein, 117 aa, synthetic construct
EVQLLESGGGLVQPGGSLRLSCAASGFTFSSYSMSWVRQAPGKGLEWVSGIGYPYYTYYADSVKGRFTISRDNSKNTLYLQMNSLRAEDTAVYYCARYYYDYGFDYWGQGTLVTVSS

SEQ ID NO: 108 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAYRSYDYVFGGGTKLTVLG

SEQ ID NO: 109 - protein, 241 aa, synthetic construct
EVQLLESGGGLVQPGGSLRLSCAASGFTFSSYSMSWVRQAPGKGLEWVSGIGYPYYTYYADSVKGRFTISRDNSKNTLYLQMNSLRAEDTAVYYCARYYYDYGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYRSYDYVFGGGTKLTVLG

SEQ ID NO: 110 - protein, 469 aa, synthetic construct
EVQLLESGGGLVQPGGSLRLSCAASGFTFSSYSMSWVRQAPGKGLEWVSGIGYPYYTYYADSVKGRFTISRDNSKNTLYLQMNSLRAEDTAVYYCARYYYDYGFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYRSYDYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - DNA, 15 bp, synthetic construct
  source 1..15: mol_type = other DNA
tcttattctatgagc

SEQ ID NO: 112 - DNA, 48 bp, synthetic construct
  source 1..48: mol_type = other DNA
ggtatcggttacccttactacacgtattacgctgattctgtaaaaggt

SEQ ID NO: 113 - DNA, 27 bp, synthetic construct
  source 1..27: mol_type = other DNA
tactactacgactacggtttcgactac

SEQ ID NO: 114 - DNA, 39 bp, synthetic construct; identical to SEQ ID NO: 14
  source 1..39: mol_type = other DNA

SEQ ID NO: 115 - DNA, 21 bp, synthetic construct
  source 1..21: mol_type = other DNA
agaaataaccagcggccaagc

SEQ ID NO: 116 - DNA, 21 bp, synthetic construct
  source 1..21: mol_type = other DNA
gctgcttaccgttcttacgac

SEQ ID NO: 117 - DNA, 351 bp, synthetic construct
  source 1..351: mol_type = other DNA
gaggtgcagctgttggagtctgggggaggcttggtacagcctggggggtccctgagactctcctgtgcagcctctggattcacctttagctcttattctatgagctgggtccgccaggctccagggaaggggctggagtgggtctcaggtatcggttacccttactacacgtattacgctgattctgtaaaaggtcggttcaccatctccagagacaattccaagaacacgctgtatctgcaaatgaacagcctgagagccgaggacacggccgtgtattactgtgcgcgttactactacgactacggtttcgactactggggccagggtacactggtcaccgtgagctca

SEQ ID NO: 118 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttaccgttcttacgactatgtcttcggcggaggcaccaagctgacggtcctaggt

SEQ ID NO: 119 - DNA, 723 bp, synthetic construct
  source 1..723: mol_type = other DNA
gaggtgcagctgttggagtctgggggaggcttggtacagcctggggggtccctgagactctcctgtgcagcctctggattcacctttagctcttattctatgagctgggtccgccaggctccagggaaggggctggagtgggtctcaggtatcggttacccttactacacgtattacgctgattctgtaaaaggtcggttcaccatctccagagacaattccaagaacacgctgtatctgcaaatgaacagcctgagagccgaggacacggccgtgtattactgtgcgcgttactactacgactacggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttaccgttcttacgactatgtcttcggcggaggcaccaagctgacggtcctaggt

SEQ ID NO: 120 - DNA, 1407 bp, synthetic construct
  source 1..1407: mol_type = other DNA
gaggtgcagctgttggagtctgggggaggcttggtacagcctggggggtccctgagactctcctgtgcagcctctggattcacctttagctcttattctatgagctgggtccgccaggctccagggaaggggctggagtgggtctcaggtatcggttacccttactacacgtattacgctgattctgtaaaaggtcggttcaccatctccagagacaattccaagaacacgctgtatctgcaaatgaacagcctgagagccgaggacacggccgtgtattactgtgcgcgttactactacgactacggtttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttaccgttcttacgactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 121 - protein, 5 aa, synthetic construct
YYYMS

SEQ ID NO: 122 - protein, 17 aa, synthetic construct
YIGGGGSGTYYADSVKG

SEQ ID NO: 123 - protein, 9 aa, synthetic construct
GPYYGYFDY

SEQ ID NO: 124 - protein, 13 aa, synthetic construct
SGSSSNIGSNYVY

SEQ ID NO: 125 - protein, 7 aa, synthetic construct
RNNQRPS

SEQ ID NO: 126 - protein, 8 aa, synthetic construct
AAYPSYDS

SEQ ID NO: 127 - protein, 118 aa, synthetic construct
EVQLLESGGGLVQPGGSLRLSCAASGFTFSYYYMSWVRQAPGKGLEWVSYIGGGGSGTYYADSVKGRFTISRDNSKNTLYLQMNSLRAEDTAVYYCARGPYYGYFDYWGQGTLVTVSS

SEQ ID NO: 128 - protein, 110 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAYPSYDSYVFGGGTKLTVLG

SEQ ID NO: 129 - protein, 243 aa, synthetic construct
EVQLLESGGGLVQPGGSLRLSCAASGFTFSYYYMSWVRQAPGKGLEWVSYIGGGGSGTYYADSVKGRFTISRDNSKNTLYLQMNSLRAEDTAVYYCARGPYYGYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYPSYDSYVFGGGTKLTVLG

SEQ ID NO: 130 - protein, 471 aa, synthetic construct
EVQLLESGGGLVQPGGSLRLSCAASGFTFSYYYMSWVRQAPGKGLEWVSYIGGGGSGTYYADSVKGRFTISRDNSKNTLYLQMNSLRAEDTAVYYCARGPYYGYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYPSYDSYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - DNA, 15 bp, synthetic construct
  source 1..15: mol_type = other DNA
tactattacatgagc

SEQ ID NO: 132 - DNA, 51 bp, synthetic construct
  source 1..51: mol_type = other DNA
tacatcggtggtggtggttctggtacgtattacgctgattctgtaaaaggt

SEQ ID NO: 133 - DNA, 27 bp, synthetic construct
  source 1..27: mol_type = other DNA
ggtccgtactacggttacttcgactac

SEQ ID NO: 134 - DNA, 39 bp, synthetic construct; identical to SEQ ID NO: 14
  source 1..39: mol_type = other DNA

SEQ ID NO: 135 - DNA, 21 bp, synthetic construct
  source 1..21: mol_type = other DNA
agaaataaccagcggccaagc

SEQ ID NO: 136 - DNA, 24 bp, synthetic construct
  source 1..24: mol_type = other DNA
gctgcttacccgtcttacgactct

SEQ ID NO: 137 - DNA, 354 bp, synthetic construct
  source 1..354: mol_type = other DNA
gaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctca

SEQ ID NO: 138 - DNA, 330 bp, synthetic construct
  source 1..330: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggt

SEQ ID NO: 139 - DNA, 729 bp, synthetic construct
  source 1..729: mol_type = other DNA
gaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggt

SEQ ID NO: 140 - DNA, 1413 bp, synthetic construct
  source 1..1413: mol_type = other DNA
gaggtgcagctgttggagtctgggggaggcttggtacagcctggggggtccctgagactctcctgtgcagcctctggattcacctttagctactattacatgagctgggtccgccaggctccagggaaggggctggagtgggtctcatacatcggtggtggtggttctggtacgtattacgctgattctgtaaaaggtcggttcaccatctccagagacaattccaagaacacgctgtatctgcaaatgaacagcctgagagccgaggacacggccgtgtattactgtgcgcgtggtccgtactacggttacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttacccgtcttacgactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 141 - protein, 5 aa, synthetic construct
YYGMS

SEQ ID NO: 142 - protein, 17 aa, synthetic construct
YISGYSSYTYYADSVKG

SEQ ID NO: 143 - protein, 9 aa, synthetic construct
YNDSGSFDY

SEQ ID NO: 144 - protein, 13 aa, synthetic construct
SGSSSNIGSNYVY

SEQ ID NO: 145 - protein, 7 aa, synthetic construct
RNNQRPS

SEQ ID NO: 146 - protein, 7 aa, synthetic construct
AADAGNR

SEQ ID NO: 147 - protein, 118 aa, synthetic construct
EVQLLESGGGLVQPGGSLRLSCAASGFTFSYYGMSWVRQAPGKGLEWVSYISGYSSYTYYADSVKGRFTISRDNSKNTLYLQMNSLRAEDTAVYYCARYNDSGSFDYWGQGTLVTVSS

SEQ ID NO: 148 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAADAGNRYVFGGGTKLTVLG

SEQ ID NO: 149 - protein, 242 aa, synthetic construct
EVQLLESGGGLVQPGGSLRLSCAASGFTFSYYGMSWVRQAPGKGLEWVSYISGYSSYTYYADSVKGRFTISRDNSKNTLYLQMNSLRAEDTAVYYCARYNDSGSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADAGNRYVFGGGTKLTVLG

SEQ ID NO: 150 - protein, 470 aa, synthetic construct
EVQLLESGGGLVQPGGSLRLSCAASGFTFSYYGMSWVRQAPGKGLEWVSYISGYSSYTYYADSVKGRFTISRDNSKNTLYLQMNSLRAEDTAVYYCARYNDSGSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAADAGNRYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - DNA, 15 bp, synthetic construct
  source 1..15: mol_type = other DNA
tactatggtatgagc

SEQ ID NO: 152 - DNA, 51 bp, synthetic construct
  source 1..51: mol_type = other DNA
tacatctctggttactcttcttacacgtattacgctgattctgtaaaaggt

SEQ ID NO: 153 - DNA, 27 bp, synthetic construct
  source 1..27: mol_type = other DNA
tacaacgactctggttctttcgactac

SEQ ID NO: 154 - DNA, 39 bp, synthetic construct; identical to SEQ ID NO: 14
  source 1..39: mol_type = other DNA

SEQ ID NO: 155 - DNA, 21 bp, synthetic construct
  source 1..21: mol_type = other DNA
agaaataaccagcggccaagc

SEQ ID NO: 156 - DNA, 21 bp, synthetic construct
  source 1..21: mol_type = other DNA
gctgctgacgctggtaaccgt

SEQ ID NO: 157 - DNA, 354 bp, synthetic construct
  source 1..354: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcatacatctctggttactcttcttacacgtattacgctgattctgtaaaaggtcggttcaccatctccagagacaattccaagaacacgctgtatctgcaaatgaacagcctgagagccgaggacacggccgtgtattactgtgcgcgttacaacgactctggttctttcgactactggggccagggtacactggtcaccgtgagctca

SEQ ID NO: 158 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacgctggtaaccgttatgtcttcggcggaggcaccaagctgacggtcctaggt

SEQ ID NO: 159 - DNA, 726 bp, synthetic construct
  source 1..726: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcatacatctctggttactcttcttacacgtattacgctgattctgtaaaaggtcggttcaccatctccagagacaattccaagaacacgctgtatctgcaaatgaacagcctgagagccgaggacacggccgtgtattactgtgcgcgttacaacgactctggttctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacgctggtaaccgttatgtcttcggcggaggcaccaagctgacggtcctaggt

SEQ ID NO: 160 - DNA, 1410 bp, synthetic construct
  source 1..1410: mol_type = other DNA
gaggtgcagctgttggagtctgggggaggcttggtacagcctggggggtccctgagactctcctgtgcagcctctggattcacctttagctactatggtatgagctgggtccgccaggctccagggaaggggctggagtgggtctcatacatctctggttactcttcttacacgtattacgctgattctgtaaaaggtcggttcaccatctccagagacaattccaagaacacgctgtatctgcaaatgaacagcctgagagccgaggacacggccgtgtattactgtgcgcgttacaacgactctggttctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgctgacgctggtaaccgttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 161 - protein, 5 aa, synthetic construct
YYDMS

SEQ ID NO: 162 - protein, 16 aa, synthetic construct
GIYGSGSTYYADSVKG

SEQ ID NO: 163 - protein, 13 aa, synthetic construct
YSYYDSYSDYFDY

SEQ ID NO: 164 - protein, 13 aa, synthetic construct
SGSSSNIGSNYVY

SEQ ID NO: 165 - protein, 7 aa, synthetic construct
RNNQRPS

SEQ ID NO: 166 - protein, 7 aa, synthetic construct
AAYSYGY

SEQ ID NO: 167 - protein, 121 aa, synthetic construct
EVQLLESGGGLVQPGGSLRLSCAASGFTFSYYDMSWVRQAPGKGLEWVSGIYGSGSTYYADSVKGRFTISRDNSKNTLYLQMNSLRAEDTAVYYCARYSYYDSYSDYFDYWGQGTLVTVSS

SEQ ID NO: 168 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAYSYGYYVFGGGTKLTVLG

SEQ ID NO: 169 - protein, 245 aa, synthetic construct
EVQLLESGGGLVQPGGSLRLSCAASGFTFSYYDMSWVRQAPGKGLEWVSGIYGSGSTYYADSVKGRFTISRDNSKNTLYLQMNSLRAEDTAVYYCARYSYYDSYSDYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYSYGYYVFGGGTKLTVLG

SEQ ID NO: 170 - protein, 473 aa, synthetic construct
EVQLLESGGGLVQPGGSLRLSCAASGFTFSYYDMSWVRQAPGKGLEWVSGIYGSGSTYYADSVKGRFTISRDNSKNTLYLQMNSLRAEDTAVYYCARYSYYDSYSDYFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYSYGYYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 15 bp, synthetic construct
  source 1..15: mol_type = other DNA
tactatgatatgagc

SEQ ID NO: 172 - DNA, 48 bp, synthetic construct
  source 1..48: mol_type = other DNA
ggtatctacggttctggttctacgtattacgctgattctgtaaaaggt

SEQ ID NO: 173 - DNA, 39 bp, synthetic construct
  source 1..39: mol_type = other DNA
tactcttactacgactcttactctgactacttcgactac

SEQ ID NO: 174 - DNA, 39 bp, synthetic construct; identical to SEQ ID NO: 14
  source 1..39: mol_type = other DNA

SEQ ID NO: 175 - DNA, 21 bp, synthetic construct
  source 1..21: mol_type = other DNA
agaaataaccagcggccaagc

SEQ ID NO: 176 - DNA, 21 bp, synthetic construct
  source 1..21: mol_type = other DNA
gctgcttactcttacggttac

SEQ ID NO: 177 - DNA, 363 bp, synthetic construct
  source 1..363: mol_type = other DNA
gaggtgcagctgttggagtctgggggaggcttggtacagcctggggggtccctgagactctcctgtgcagcctctggattcacctttagctactatgatatgagctgggtccgccaggctccagggaaggggctggagtgggtctcaggtatctacggttctggttctacgtattacgctgattctgtaaaaggtcggttcaccatctccagagacaattccaagaacacgctgtatctgcaaatgaacagcctgagagccgaggacacggccgtgtattactgtgcgcgttactcttactacgactcttactctgactacttcgactactggggccagggtacactggtcaccgtgagctca

SEQ ID NO: 178 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactcttacggttactatgtcttcggcggaggcaccaagctgacggtcctaggt

SEQ ID NO: 179 - DNA, 735 bp, synthetic construct
  source 1..735: mol_type = other DNA
gaggtgcagctgttggagtctgggggaggcttggtacagcctggggggtccctgagactctcctgtgcagcctctggattcacctttagctactatgatatgagctgggtccgccaggctccagggaaggggctggagtgggtctcaggtatctacggttctggttctacgtattacgctgattctgtaaaaggtcggttcaccatctccagagacaattccaagaacacgctgtatctgcaaatgaacagcctgagagccgaggacacggccgtgtattactgtgcgcgttactcttactacgactcttactctgactacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactcttacggttactatgtcttcggcggaggcaccaagctgacggtcctaggt

SEQ ID NO: 180 - DNA, 1419 bp, synthetic construct
  source 1..1419: mol_type = other DNA
gaggtgcagctgttggagtctgggggaggcttggtacagcctggggggtccctgagactctcctgtgcagcctctggattcacctttagctactatgatatgagctgggtccgccaggctccagggaaggggctggagtgggtctcaggtatctacggttctggttctacgtattacgctgattctgtaaaaggtcggttcaccatctccagagacaattccaagaacacgctgtatctgcaaatgaacagcctgagagccgaggacacggccgtgtattactgtgcgcgttactcttactacgactcttactctgactacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactcttacggttactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 181 - protein, 5 aa, synthetic construct
SYGMS

SEQ ID NO: 182 - protein, 17 aa, synthetic construct
SIYYYSGGTYYADSVKG

SEQ ID NO: 183 - protein, 11 aa, synthetic construct
NDYRNNSDFDY

SEQ ID NO: 184 - protein, 13 aa, synthetic construct
SGSSSNIGSNYVY

SEQ ID NO: 185 - protein, 7 aa, synthetic construct
RNNQRPS

SEQ ID NO: 186 - protein, 7 aa, synthetic construct
AAYYPYY

SEQ ID NO: 187 - protein, 120 aa, synthetic construct
EVQLLESGGGLVQPGGSLRLSCAASGFTFSSYGMSWVRQAPGKGLEWVSSIYYYSGGTYYADSVKGRFTISRDNSKNTLYLQMNSLRAEDTAVYYCARNDYRNNSDFDYWGQGTLVTVSS

SEQ ID NO: 188 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAYYPYYYVFGGGTKLTVLG

SEQ ID NO: 189 - protein, 244 aa, synthetic construct
EVQLLESGGGLVQPGGSLRLSCAASGFTFSSYGMSWVRQAPGKGLEWVSSIYYYSGGTYYADSVKGRFTISRDNSKNTLYLQMNSLRAEDTAVYYCARNDYRNNSDFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYYPYYYVFGGGTKLTVLG

SEQ ID NO: 190 - protein, 472 aa, synthetic construct
EVQLLESGGGLVQPGGSLRLSCAASGFTFSSYGMSWVRQAPGKGLEWVSSIYYYSGGTYYADSVKGRFTISRDNSKNTLYLQMNSLRAEDTAVYYCARNDYRNNSDFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAAYYPYYYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - DNA, 15 bp, synthetic construct
  source 1..15: mol_type = other DNA
tcttatggtatgagc

SEQ ID NO: 192 - DNA, 51 bp, synthetic construct
  source 1..51: mol_type = other DNA
tctatctactactactctggtggtacgtattacgctgattctgtaaaaggt

SEQ ID NO: 193 - DNA, 33 bp, synthetic construct
  source 1..33: mol_type = other DNA
aacgactaccgtaacaactctgacttcgactac

SEQ ID NO: 194 - DNA, 39 bp, synthetic construct; identical to SEQ ID NO: 14
  source 1..39: mol_type = other DNA

SEQ ID NO: 195 - protein, 21 aa, synthetic construct
AGAAATAACCAGCGGCCAAGC

SEQ ID NO: 196 - DNA, 21 bp, synthetic construct
  source 1..21: mol_type = other DNA
gctgcttactacccgtactac

SEQ ID NO: 197 - DNA, 360 bp, synthetic construct
  source 1..360: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctca

SEQ ID NO: 198 - DNA, 327 bp, synthetic construct
  source 1..327: mol_type = other DNA
cagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggt

SEQ ID NO: 199 - DNA, 732 bp, synthetic construct
  source 1..732: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggt

SEQ ID NO: 200 - DNA, 1416 bp, synthetic construct
  source 1..1416: mol_type = other DNA
gaggtgcagctgttggagtctgggggaggcttggtacagcctggggggtccctgagactctcctgtgcagcctctggattcacctttagctcttatggtatgagctgggtccgccaggctccagggaaggggctggagtgggtctcatctatctactactactctggtggtacgtattacgctgattctgtaaaaggtcggttcaccatctccagagacaattccaagaacacgctgtatctgcaaatgaacagcctgagagccgaggacacggccgtgtattactgtgcgcgtaacgactaccgtaacaactctgacttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgcttactacccgtactactatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 201 - protein, 5 aa, synthetic construct
SYHMS

SEQ ID NO: 202 - protein, 17 aa, synthetic construct
GIGYGHGGTYYADSVKG

SEQ ID NO: 203 - protein, 9 aa, synthetic construct
YDYDYSFDY

SEQ ID NO: 204 - protein, 13 aa, synthetic construct
SGSSSNIGSNYVY

SEQ ID NO: 205 - protein, 7 aa, synthetic construct
RNNQRPS

SEQ ID NO: 206 - protein, 7 aa, synthetic construct
AVSSPYS

SEQ ID NO: 207 - protein, 118 aa, synthetic construct
EVQLLESGGGLVQPGGSLRLSCAASGFTFSSYHMSWVRQAPGKGLEWVSGIGYGHGGTYYADSVKGRFTISRDNSKNTLYLQMNSLRAEDTAVYYCARYDYDYSFDYWGQGTLVTVSS

SEQ ID NO: 208 - protein, 109 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAVSSPYSYVFGGGTKLTVLG

SEQ ID NO: 209 - protein, 242 aa, synthetic construct
EVQLLESGGGLVQPGGSLRLSCAASGFTFSSYHMSWVRQAPGKGLEWVSGIGYGHGGTYYADSVKGRFTISRDNSKNTLYLQMNSLRAEDTAVYYCARYDYDYSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAVSSPYSYVFGGGTKLTVLG

SEQ ID NO: 210 - protein, 470 aa, synthetic construct
EVQLLESGGGLVQPGGSLRLSCAASGFTFSSYHMSWVRQAPGKGLEWVSGIGYGHGGTYYADSVKGRFTISRDNSKNTLYLQMNSLRAEDTAVYYCARYDYDYSFDYWGQGTLVTVSSGGGGSGGGGSGGGGSQSVLTQPPSASGTPGQRVTISCSGSSSNIGSNYVYWYQQLPGTAPKLLIYRNNQRPSGVPDRFSGSKSGTSASLAISGLRSEDEADYYCAVSSPYSYVFGGGTKLTVLGGQAGQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 211 - DNA, 15 bp, synthetic construct
  source 1..15: mol_type = other DNA
tcttatcacatgagc

SEQ ID NO: 212 - DNA, 51 bp, synthetic construct
  source 1..51: mol_type = other DNA
ggtatcggttacggtcacggtggtacgtattacgctgattctgtaaaaggt

SEQ ID NO: 213 - DNA, 27 bp, synthetic construct
  source 1..27: mol_type = other DNA
tacgactacgactactctttcgactac

SEQ ID NO: 214 - DNA, 39 bp, synthetic construct
  source 1..39: mol_type = other DNA
tcctgctcttcatctaatattggctctaattatgtctac

SEQ ID NO: 215 - DNA, 21 bp, synthetic construct
  source 1..21: mol_type = other DNA
agaaataaccagcggccaagc

SEQ ID NO: 216 - DNA, 21 bp, synthetic construct
  source 1..21: mol_type = other DNA
gctgtttcttctccgtactct

SEQ ID NO: 217 - DNA, 354 bp, synthetic construct
  source 1..354: mol_type = other DNA
gaggtgcagctgttggagtctgggggaggcttggtacagcctggggggtccctgagactctcctgtgcagcctctggattcacctttagctcttatcacatgagctgggtccgccaggctccagggaaggggctggagtgggtctcaggtatcggttacggtcacggtggtacgtattacgctgattctgtaaaaggtcggttcaccatctccagagacaattccaagaacacgctgtatctgcaaatgaacagcctgagagccgaggacacggccgtgtattactgtgcgcgttacgactacgactactctttcgactactggggccagggtacactggtcaccgtgagctca

SEQ ID NO: 218 - DNA, 327 bp, synthetic construct
  source 1..327: mol_type = other DNA
cagtctgtgctgactcagccaccctcagcgtctgggacccccgggcagagggtcaccatctcttgttcctgctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgtttcttctccgtactcttatgtcttcggcggaggcaccaagctgacggtcctaggt

SEQ ID NO: 219 - DNA, 726 bp, synthetic construct
  source 1..726: mol_type = other DNA
gaggtgcagctgttggagtctgggggaggcttggtacagcctggggggtccctgagactctcctgtgcagcctctggattcacctttagctcttatcacatgagctgggtccgccaggctccagggaaggggctggagtgggtctcaggtatcggttacggtcacggtggtacgtattacgctgattctgtaaaaggtcggttcaccatctccagagacaattccaagaacacgctgtatctgcaaatgaacagcctgagagccgaggacacggccgtgtattactgtgcgcgttacgactacgactactctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgtttcttctccgtactcttatgtcttcggcggaggcaccaagctgacggtcctaggt

SEQ ID NO: 220 - DNA, 1410 bp, synthetic construct
  source 1..1410: mol_type = other DNA
gaggtgcagctgttggagtctgggggaggcttggtacagcctggggggtccctgagactctcctgtgcagcctctggattcacctttagctcttatcacatgagctgggtccgccaggctccagggaaggggctggagtgggtctcaggtatcggttacggtcacggtggtacgtattacgctgattctgtaaaaggtcggttcaccatctccagagacaattccaagaacacgctgtatctgcaaatgaacagcctgagagccgaggacacggccgtgtattactgtgcgcgttacgactacgactactctttcgactactggggccagggtacactggtcaccgtgagctcaggtggaggcggttcaggcggaggtggatccggcggtggcggatcgcagtctgtgctgactcagccaccctcagcgtctgggacccccgggcagagggtcaccatctcttgtagtggctcttcatctaatattggctctaattatgtctactggtaccagcagctcccaggaacggcccccaaactcctcatctatagaaataaccagcggccaagcggggtccctgaccgattctctggctccaagtctggcacctcagcctccctggccatcagtgggctccggtccgaggatgaggccgattattactgtgctgtttcttctccgtactcttatgtcttcggcggaggcaccaagctgacggtcctaggtggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 221 - DNA, 1184 bp, synthetic construct
  source 1..1184: mol_type = other DNA
tgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 222 - DNA, 684 bp, synthetic construct
  source 1..684: mol_type = other DNA
ggccaggccggccagacaacgacacctgctcccagaccgcctactcccgccccaaccattgcatctcagccactctctctgagacccgaagcgtgtagacctgcggccgggggcgctgtccacacaagaggcttagacttcgcctgcgatatctatatctgggccccactcgcaggcacttgtggagtgctgctgctttcactcgtgataaccctgtactgcaaaagggggagaaagaagctgctgtatatttttaaacaaccatttatgagacctgttcagactacccaggaagaagacggttgtagttgcagattccccgaggaggaagaaggaggttgcgagttgagagtaaagttcagcagatccgcagatgcccctgcttaccagcagggtcaaaaccagctttacaacgagctgaatttaggtagaagagaggaatatgacgtgttggataaaagaagaggaagagacccggaaatgggcggcaagcctcgaagaaaaaatccccaagagggactctacaatgagctgcagaaggacaaaatggctgaagcctacagcgagatcggcatgaagggagaaagacgcagagggaaagggcatgatgggctttatcagggcttgtccaccgctacaaaggatacttatgacgcactacacatgcaggccctgccaccccgt

SEQ ID NO: 223 - protein, 361 aa, synthetic construct
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 224 - protein, 226 aa, synthetic construct
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQC

SEQ ID NO: 225 - protein, 226 aa, synthetic construct
PGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

SEQ ID NO: 226 - protein, 270 aa, synthetic construct
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGK

(19) United States
(12) Patent Application Publication
(10) Pub. No.: US 2024/0392023 A1
(43) Pub. Date: Nov. 28, 2024

(54) ANTI-CD30 ANTIBODY AND CHIMERIC ANTIGEN RECEPTOR COMPRISING THEREOF

(71) Applicants: ABCLON INC., Seoul (KR); The Trustees of the University of Pennsylvania, Philadelphia, PA (US)

(72) Inventors: Jong-Seo LEE, Gyeonggi-do (KR); Jong-Hoon KIM, Gyeonggi-do (KR); Ki Hyun KIM, Seoul (KR); Hyun-Jong LEE, Incheon (KR); Marco RUELLA, Ardmore, PA (US); Puneeth GURUPRASAD, Philadelphia, PA (US)

(21) Appl. No.: 18/198,937

(22) Filed: May 18, 2023

(51) Int. Cl. C07K 16/28; A61K 39/00; C07K 14/705; C07K 14/725

(52) U.S. Cl. CPC C07K 16/2878; A61K 39/4631; C07K 14/7051; C07K 14/70517; C07K 14/70578; A61K 2039/505; A61K 2239/13; A61K 2239/21; A61K 2239/22; C07K 2317/622; C07K 2317/73; C07K 2317/92; C07K 2319/02; C07K 2319/03; C07K 2319/33

(57) ABSTRACT

Disclosed herein are a novel antibody and an antigen binding fragment thereof that targets CD30 to treat cancer, a chimeric antigen receptor, and a use thereof. The antibody, which binds specifically to CD30 that is highly expressed in cancer cells (specifically blood cancer), is very low in sequence homology with the CDR sequences of conventional CD30 targeting antibodies and thus has a unique sequence. Cells that express the chimeric antigen receptor including the anti-CD30 antibody or the antigen binding fragment responds to CD30-expressing positive cells to induce immune cell activity, thus finding advantageous applications as a CAR-immune cell product.

### SEQUENCE LISTING

This application contains references to amino acid sequences and/or nucleic acid sequences which have been submitted concurrently herewith as the sequence listing XML file entitled “000341 us_SequenceListing.XML”, file size 236 kilobytes, created on 18 May 2023. The aforementioned sequence listing is hereby incorporated by reference in its entirety pursuant to 37 C.F.R. § 1.52(e)(5).

### FIELD

The present disclosure relates to a novel antibody or an antigen binding fragment thereof for use in targeting CD30 to treat cancer, a chimeric antigen receptor including same, and uses thereof.

### BACKGROUND

B cell malignancies are tumors generated in B cells, which are a type of cell lineage responsible for the immune system of the body. Such a B cell malignancy breaks a normal immune system to decrease the immunity against antigens invading from the outside, finally causing the death of patients. For example, acute lymphocytic leukemia (ALL), which is one of B cell malignancies, refers to a disease in which the lymphoid line of white blood cells becomes malignant, grows in the bone marrow, and spreads to peripheral blood, thus invading the liver, the spleen, the lymph, the cerebrum, the cerebellum, the spinal cord, and so on. Acute lymphocytic leukemia is predicted to have a global incidence of 161,000 and a death number of 110,000 in 2015, and both outbreaks of acute lymphocytic leukemia and deaths from acute lymphocytic leukemia are more prevalent in men than women. Representative of therapies for acute lymphocytic leukemia are chemotherapy, targeted therapy, and allogeneic stem cell transplantation. These therapies have been improved to carry the survival rate of child patients to over 85%.

CD30 is attracting attention as a target for these diseases. The CD30 cell surface molecule is a member of the tumor necrosis factor receptor (TNF-R) superfamily. CD30 is a type I transmembrane glycoprotein with six (human) or three (murine and rat) cysteine-rich repeats with a central hinge sequence. CD30 exists as a 120 kDa membrane molecule which develops from an intercellular precursor protein of 90 kDa.

CD30 is preferentially expressed by activated lymphoid cells. Specifically, stimulation of CD30 in lymphoid cells has been shown to induce pleiotropic biological effects, including proliferation, activation, differentiation, and cell death, depending on cell type, stage of differentiation and presence of other stimuli. CD30 was originally identified by the monoclonal antibody Ki-1, which is reactive with antigens expressed on Hodgkin and Reed-Sternberg cells of Hodgkin's disease. Accordingly, CD30 is widely used as a clinical marker for Hodgkin's lymphoma and related hematological malignancies.

CD30 was subsequently shown to be expressed on a subset of non-Hodgkin's lymphomas (NHL), including Burkitt's lymphoma, anaplastic large-cell lymphomas (ALCL), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), and entroblastic/centrocytic (cb/cc) follicular lymphomas, as well as several virally-transformed lines such as human T-Cell Lymphotrophic Virus I or II transformed T-cells, and Epstein-Barr Virus transformed B-cells.

Since the percentage of CD30-positive cells in normal individuals is quite small, the expression of CD30 in tumor cells renders it an important target for antibody mediated therapy to specifically target therapeutic agents against CD30-positive neoplastic cells. However, while the results obtained to date clearly establish CD30 as a useful target for immunotherapy, they also show that currently available murine antibodies do not constitute ideal therapeutic agents. Accordingly, the need exists for anti-CD antibodies effective for treating or preventing diseases related with CD30-expressing cells.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a plot quantitatively analyzing binding affinity of 11 Fc-linked antibodies for CD30.

FIG. 2 shows graphs quantitatively analyzing the binding affinity of the CD30-binding antibodies for CD30-overexpressing CD30-293T cells.

FIG. 3 is a graph showing the specific binding affinity of the 11 antibodies for CD30 as measured by ELISA for the TNFR family members including CD30 (CD27, CD270, CD134, CD137, CD357, and CD30).

FIG. 4 shows the binding of the 11 antibodies to the chimeric extracellular domains of CD30 protein (CD30 (F), CD30 (D1L), CD30 (LD2), and CD30 (D1D2)) to identify a binding site on the extracellular domain of CD30 protein (A. schematic view of CD30 chimeric extracellular domain protein, B. data of binding to chimeric extracellular domain protein as measured by ELISA).

FIG. 5 is a schematic view of CAR structures including an CD30 antibody and an HRS antibody used as a control.

FIG. 6 shows cell proliferation (A) and viability (B) of CAR-T including CD30 antibodies.

FIG. 7 shows results of an assay for cell proliferation ability of CD30 CAR-T containing T1-159.

FIG. 8 shows ratios of expressing anti-CD30 CAR in the CAR-T constructed in the present disclosure.

FIG. 9 shows plots of cytotoxicity effects of CD30-CAR-T on the CD30-positive cells SUDHL1 (A) and CD30-293T (B).

FIG. 10 shows results of an assay for tumor death effects of CD30 CAR-T containing T1-36 or T1-159.

FIG. 11 shows the presence or absence of an off-target effect of the CD30 CAR-T containing T1-159 by examining whether CD30 CAR-T containing T1-159 has cell death effects on CD30⁻ cells.

FIG. 12 are plots of IFN release from CD30-CAR-T for the CD30-positive cells SUDHL1 (A) and CD30-293T (B).

FIG. 13 shows results of an assay for anticancer activity of T1-159 against HDLM-2 cell-implanted NSG mice as measured for tumor volumes.

### DETAILED DESCRIPTION

The present inventors have conducted intensive and thorough research into development of a novel antibody specifically binding to CD30 and a chimeric antigen receptor using the same, in order to treat tumorigenic diseases and immune diseases. As a result, the present inventors have discovered, among other things, that an anti-CD30 antibody or an antigen-binding fragment thereof that specifically binds to CD30 antigen or an effector cell expressing a chimeric antigen receptor comprising same retains cytotoxic activity and antitumoral effects, which leads to the present disclosure.

Therefore, an aspect of the present disclosure is to provide an anti-CD30 antibody or an antigen-binding fragment thereof.

Another aspect of the present disclosure is to provide a nucleic acid molecule encoding the anti-CD30 antibody or the antigen-binding fragment thereof.

Another aspect of the present disclosure is to provide a CD30-specific chimeric antigen receptor comprising: an extracellular domain comprising an anti-CD30 antibody or an antigen-binding fragment thereof; a transmembrane domain; and an intracellular signaling domain.

In an aspect thereof, the present disclosure provides an anti-CD30 antibody or an antigen binding fragment thereof, comprising an immunoglobulin heavy-chain variable region domain and an immunoglobulin light-chain variable region domain, wherein:
i) the heavy-chain variable region domain comprises HCDR1, HCDR2, and HCDR3, wherein the HCDR1, HCDR2, and HCDR3 comprise the amino acid sequence of SEQ ID NO: 1, 2, and 3, respectively; and the light-chain variable region domain comprises LCDR1, LCDR2, and LCDR3, wherein the LCDR1, LCDR2, and LCDR3 comprise the amino acid sequence of SEQ ID NO: 4, 5, and 6, respectively;
ii) the heavy-chain variable region domain comprises HCDR1, HCDR2, and HCDR3, wherein the HCDR1, HCDR2, and HCDR3 comprise the amino acid sequence of SEQ ID NO: 21, 22, and 23, respectively; and the light-chain variable region domain comprises LCDR1, LCDR2, and LCDR3, wherein the LCDR1, LCDR2, and LCDR3 comprise the amino acid sequence of SEQ ID NO: 24, 25, and 26, respectively;
iii) the heavy-chain variable region domain comprises HCDR1, HCDR2, and HCDR3, wherein the HCDR1, HCDR2, and HCDR3 comprise the amino acid sequence of SEQ ID NO: 41, 42, and 43, respectively; and the light-chain variable region domain comprises LCDR1, LCDR2, and LCDR3, wherein the LCDR1, LCDR2, and LCDR3 comprise the amino acid sequence of SEQ ID NO: 44, 45, and 46, respectively;
iv) the heavy-chain variable region domain comprises HCDR1, HCDR2, and HCDR3, wherein the HCDR1, HCDR2, and HCDR3 comprise the amino acid sequence of SEQ ID NO: 61, 62, and 63, respectively; and the light-chain variable region domain comprises LCDR1, LCDR2, and LCDR3, wherein the LCDR1, LCDR2, and LCDR3 comprise the amino acid sequence of SEQ ID NO: 64, 65, and 66, respectively;
v) the heavy-chain variable region domain comprises HCDR1, HCDR2, and HCDR3, wherein the HCDR1, HCDR2, and HCDR3 comprise the amino acid sequence of SEQ ID NO: 81, 82, and 83, respectively; and the light-chain variable region domain comprises LCDR1, LCDR2, and LCDR3, wherein the LCDR1, LCDR2, and LCDR3 comprise the amino acid sequence of SEQ ID NO: 84, 85, and 86, respectively;
vi) the heavy-chain variable region domain comprises HCDR1, HCDR2, and HCDR3, wherein the HCDR1, HCDR2, and HCDR3 comprise the amino acid sequence of SEQ ID NO: 101, 102, and 103, respectively; and the light-chain variable region domain comprises LCDR1, LCDR2, and LCDR3, wherein the LCDR1, LCDR2, and LCDR3 comprise the amino acid sequence of SEQ ID NO: 104, 105, and 106, respectively;
vii) the heavy-chain variable region domain comprises HCDR1, HCDR2, and HCDR3, wherein the HCDR1, HCDR2, and HCDR3 comprise the amino acid sequence of SEQ ID NO: 121, 122, and 123, respectively; and the light-chain variable region domain comprises LCDR1, LCDR2, and LCDR3, wherein the LCDR1, LCDR2, and LCDR3 comprise the amino acid sequence of SEQ ID NO: 124, 125, and 126, respectively;
viii) the heavy-chain variable region domain comprises HCDR1, HCDR2, and HCDR3, wherein the HCDR1, HCDR2, and HCDR3 comprise the amino acid sequence of SEQ ID NO: 141, 142, and 143, respectively; and the light-chain variable region domain comprises LCDR1, LCDR2, and LCDR3, wherein the LCDR1, LCDR2, and LCDR3 comprise the amino acid sequence of SEQ ID NO: 144, 145, and 146, respectively;
ix) the heavy-chain variable region domain comprises HCDR1, HCDR2, and HCDR3, wherein the HCDR1, HCDR2, and HCDR3 comprise the amino acid sequence of SEQ ID NO: 161, 162, and 163, respectively; and the light-chain variable region domain comprises LCDR1, LCDR2, and LCDR3, wherein the LCDR1, LCDR2, and LCDR3 comprise the amino acid sequence of SEQ ID NO: 164, 165, and 166, respectively;
x) the heavy-chain variable region domain comprises HCDR1, HCDR2, and HCDR3, wherein the HCDR1, HCDR2, and HCDR3 comprise the amino acid sequence of SEQ ID NO: 181, 182, and 183, respectively; and the light-chain variable region domain comprises LCDR1, LCDR2, and LCDR3, wherein the LCDR1, LCDR2, and LCDR3 comprise the amino acid sequence of SEQ ID NO: 184, 185, and 186, respectively; or
xi) the heavy-chain variable region domain comprises HCDR1, HCDR2, and HCDR3, wherein the HCDR1, HCDR2, and HCDR3 comprise the amino acid sequence of SEQ ID NO: 201, 202, and 203, respectively; and the light-chain variable region domain comprises LCDR1, LCDR2, and LCDR3, wherein the LCDR1, LCDR2, and LCDR3 comprise the amino acid sequence of SEQ ID NO: 204, 205, and 206, respectively.

Intensive and thorough research conducted by the present inventors into the development of a novel antibody binding specifically to CD30 and a chimeric antigen receptor using same for use in treating tumorigenic diseases or immune diseases resulted in the finding that the anti-CD30 antibody binding selectively to the CD30 antigen and an effector cell expressing a chimeric antigen receptor including a fragment of the antibody bring about cytolytic and antitumor effects.

As used herein, the term “CD30”, also known as a member of the TNF receptor (TNF-R) superfamily, refers to a cell membrane protein of the tumor necrosis factor receptor family. This family of molecules has variable homology among its members and includes nerve growth factor receptor (NGFR), CD120 (a), CD120 (b), CD27, CD40, and CD95. These molecules are typically characterized by the presence of multiple cysteine-rich repeats in the extracytoplasmic region. Members of this family are considered crucial for regulating proliferation and differentiation of lymphocytes.

As used herein, the term “antibody” used in context of CD30 refers to an antibody specific for CD30 and is intended to encompass not only a whole antibody form, but also an antigen-binding fragment thereof.

A whole antibody includes two full length light chain and two full length heavy chains where each light chain is linked to the heavy chain by disulfide bonds. The heavy chain constant region is divided into isotypes of gamma (γ), mu (μ), alpha (α), delta (δ), and epsilon (ε) types, which are further subtyped into gamma1 (γ1), gamma2 (γ2), gamma3 (γ3), gamma4 (γ4), alpha 1 (α1), and alpha 2 (α2). The light chain constant region is divided into kappa (κ) and lambda (λ) types.

As used herein, the term “antigen-binding fragment” refers to a fragment retaining the function of binding to an antigen and includes Fab, F(ab′), F(ab′)₂, and Fv. Of them, Fab (fragment antigen binding) is composed of one constant and one variable domain of each of the heavy and the light chain, the constant domain of the heavy chain being the first constant domain (CH1), and thus contains one antigen-binding site. Fab′ is different from Fab in that the former comprises a hinge region including at least one cysteine residue at the C-terminal of the CH1 domain of a heavy chain. F(ab′)₂ is produced by a disulfide bond formation between cysteine residues in the hinge region of Fab′. Fv is an antibody fragment composed only of variable regions of a heavy and a light chain, which may be produced by a recombinant technology disclosed in the art. In Fv (two-chain Fv), variable regions of a light and heavy chain are linked by a non-covalent bond, and in a single chain Fv, variable regions of a light and heavy chain are linked by a covalent bond through a peptide linker or it may form a dimer structure like a two chain FV through a direct linkage at the C-terminal. These antibody fragments can be obtained through a proteinase treatment (for example, a whole antibody may be treated with a papain to obtain Fab fragments or with pepsin to obtain F(ab′)₂ fragment) or preferably constructed using a recombinant DNA technology.

Herein, examples of the antibody include a monoclonal antibody, a multispecific antibody, a human antibody, a humanized antibody, a chimeric antibody, a single-chain Fv (scFv), a single-chain antibody, an Fab, an F(ab′), a disulfide-linked Fv (sdFv), an anti-idiotype (anti-Id) antibody, and an epitope-binding fragment thereof, but are not limited thereto.

The term “heavy chain”, as used herein, refers to a full-length chain comprising three constant regions CH1, CH2 and CH3 and one variable region VH comprising an amino acid sequence which is sufficient for conferring specificity to an antigen as well fragments thereof. Also the term “light chain” as used herein refers to a full length chain comprising one constant region CL and one variable region VL comprising an amino acid sequence which is sufficient for conferring specificity to an antigen as well fragments thereof.

The term “variable region” or “variable domain”, as used herein, refers to a domain on a heavy or a light chain of an antibody, which is responsible for binding the antibody to an antigen. Variable domains on the heavy and the light chain of a native antibody (VH and VL, respectively) are generally similar in structure and each include four conserved framework regions (FRs) and three hypervariable regions (HVRs) (Kindt et al., Kuby Immunology, 6^{th} edition, W. H. Freeman and Co., page 91 (2007)).

As used herein, the term “CDR” (complementarity determining region) refers to an amino acid sequence of the hypervariable regions on the immunoglobulin heavy and light chains (Kabat et al., Sequences of Proteins of Immunological Interest, 4th Ed., U. S. Department of Health and Human Services, National Institutes of Health (1987)). Three CDRs are included in each of the heavy chain (CDRH1, CDRH2, and CDRH3) and the light chain (CDRL1, CDRL2, and CDRL3). CDRs provide important contact residues with which the antibody binds to an antigen or an epitope.

As used herein, the term “framework” or “FR” refers to variable domain residues other than hypervariable region (HVR) residues. The FR of the variable domain generally consists of four FR domains: FR1, FR2, FR3, and FR4. Thus, the HVR and FR sequences generally appear in the following sequence in VH:

FRH1 (framework region 1 of heavy chain)-CDRH1 (complementarity determining region 1 of heavy chain)-FRH2-CDRH2-FRH3-CDRH3-FRH4.

Also, HVR and FR sequences in VL (or Vk) are arranged in the order as follows: FRL1 (framework region 1 of light chain)-CDRL1 (complementarity determining region 1 of light chain)-FRL2-CDRL2-FRL3-CDRL3-FRL4.

By the term “specifically binding” or wordings relevant thereto, it is intended that an antibody or a constituent thereof, such as an antigen binding fragment or scFv, forms a complex with an antigen that is relatively stable under physiologic conditions. Specific binding can be characterized by an equilibrium dissociation constant of at least about 1×10⁻⁶ M or less (e.g., less KD means more strong binding). Methods for determining whether two molecules specifically bind are well known in the art and include, for example, equilibrium dialysis, surface plasmon resonance, and the like.

The term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

The term “human antibody” or “humanized antibody”, as used herein, refers to an antibody which possesses an amino acid sequence which corresponds to that of an antibody produced by a human or a human cell or derived from a non-human source that utilizes human antibody repertoires or other human antibody-encoding sequences.

As used herein, the term “chimeric antibody” refers to an antibody in which a portion of the heavy and/or light chain is derived from a particular source or species, while the remainder of the heavy and/or light chain is derived from a different source or species.

In the context of specifically recognizing CD30, variants of the amino acid sequences, as recognized by a person skilled in the art, may fall within the scope of the anti-CD30 antibody or an antigen-binding fragment thereof according to the present disclosure. For example, a variation may be given to the amino acid sequence of an antibody in order to improve the binding affinity and/or other biological properties of the antibody. The variation includes a deletion, an addition, and/or a substitution of an amino acid residue on the amino acid sequence of the antibody.

Such amino acid variations may be provided on the basis of a relative similarity of amino acid side chains, e.g., hydrophobicity, hydrophilicity, charge, and size. As analyzed for size, shape, and type of amino acid side chains, it is clear that all of arginine, lysine, and histidine residues are positively charged; alanine, glycine, and serine are similar in size; phenylalanine, tryptophan, and tyrosine have similar shapes. Accordingly, based on this consideration, arginine, lysine and histidine; alanine, glycine and serine; and phenylalanine, tryptophan and tyrosine may be considered to be biologically functional equivalents.

In making such variations, the hydropathic index of amino acids may be considered. Each amino acid has been assigned a hydropathic index on the basis of their hydrophobicity and charge characteristics: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art. It is known that similar biological activity is retained only upon substitution of certain amino acids for other amino acids having a similar hydropathic index. In making variations based upon the hydropathic index, the substitution of amino acids whose hydropathic indices are within ±2 is preferred, those which are within ±1 are particularly preferred, and those within ±0.5 are even more particularly preferred.

It is also understood in the art that substitutions between amino acids having similar hydrophilicity values may result in the generation of proteins having biologically equivalent activities. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4).

In making variations based upon similar hydrophilicity values, the substitution of amino acids whose hydrophilicity values are within ±2 is preferred, those which are within ±1 are particularly preferred, and those within ±0.5 are even more particularly preferred.

The amino acid exchanges in proteins that do not substantially change the activity of the molecule are well known in the art (H. Neurath, R. L. Hill, The Proteins, Academic Press, New York, 1979). The most common exchanges are found between amino acid residues: Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In an embodiment of the present disclosure, the anti-CD30 antibody or the antigen binding fragment thereof comprises a heavy chain variable region domain and a light chain variable region domain, wherein:
i) the heavy-chain variable region domain comprises the amino acid sequence of SEQ ID NO: 7; and the light-chain variable region domain comprises the amino acid sequence of SEQ ID NO: 8;
ii) the heavy-chain variable region domain comprises the amino acid sequence of SEQ ID NO: 27; and the light-chain variable region domain comprises the amino acid sequence of SEQ ID NO: 28;
iii) the heavy-chain variable region domain comprises the amino acid sequence of SEQ ID NO: 47; and the light-chain variable region domain comprises the amino acid sequence of SEQ ID NO: 48;
iv) the heavy-chain variable region domain comprises the amino acid sequence of SEQ ID NO: 67; and the light-chain variable region domain comprises the amino acid sequence of SEQ ID NO: 68;
v) the heavy-chain variable region domain comprises the amino acid sequence of SEQ ID NO: 87; and the light-chain variable region domain comprises the amino acid sequence of SEQ ID NO: 88;
vi) the heavy-chain variable region domain comprises the amino acid sequence of SEQ ID NO: 107; and the light-chain variable region domain comprises the amino acid sequence of SEQ ID NO: 108;
vii) the heavy-chain variable region domain comprises the amino acid sequence of SEQ ID NO: 127; and the light-chain variable region domain comprises the amino acid sequence of SEQ ID NO: 128;
viii) the heavy-chain variable region domain comprises the amino acid sequence of SEQ ID NO: 147; and the light-chain variable region domain comprises the amino acid sequence of SEQ ID NO: 148;
ix) the heavy-chain variable region domain comprises the amino acid sequence of SEQ ID NO: 167; and the light-chain variable region domain comprises the amino acid sequence of SEQ ID NO: 168;
x) the heavy-chain variable region domain comprises the amino acid sequence of SEQ ID NO: 187; and the light-chain variable region domain comprises the amino acid sequence of SEQ ID NO: 188; or
xi) the heavy-chain variable region domain comprises the amino acid sequence of SEQ ID NO: 207; and the light-chain variable region domain comprises the amino acid sequence of SEQ ID NO: 208.

In an embodiment of the present disclosure, the anti-CD30 antibody or the antigen binding fragment thereof comprises scFv, wherein the scFv comprises an amino acid sequence selected from the group consisting of the amino acid sequence of SEQ ID NO: 9, the amino acid sequence of SEQ ID NO: 29, the amino acid sequence of SEQ ID NO: 49, the amino acid sequence of SEQ ID NO: 69, the amino acid sequence of SEQ ID NO: 89, the amino acid sequence of SEQ ID NO: 109, the amino acid sequence of SEQ ID NO: 129, the amino acid sequence of SEQ ID NO: 149, the amino acid sequence of SEQ ID NO: 169, the amino acid sequence of SEQ ID NO: 189, and the amino acid sequence of SEQ ID NO: 209.

In an embodiment of the present disclosure, the antigen binding fragment is Fab, Fab′, F(ab′)₂, Fv, scFV, or chemically linked F(ab′) 2.

An aspect of the present invention provides a nucleic acid molecule coding for the anti-CD30 antibody or an antigen-binding fragment thereof.

The term “nucleic acid molecule”, as used herein, is intended to encompass DNA (gDNA and cDNA) and RNA molecules. Nucleotides are the basic building block of the nucleic acid molecule and include sugar or base-modified analogues as well as natural nucleotides (Scheit, Nucleotide Analogs, John Wiley, New York (1980); Uhlman and Peyman, Chemical Reviews, 90:543-584 (1990)).

It should be understood to a person skilled in the art that the nucleotide sequence coding for the antibody, the antigen-biding fragment thereof, or the chimeric antigen receptor polypeptide according to the present disclosure is any nucleotide sequence that encode an amino acid sequence constituting the chimeric antigen receptor molecule and is not limited to particular nucleotide sequences.

The reason is that even if the nucleotide sequence undergoes mutation, the expression of the mutated nucleotide sequence into a protein may not cause a change in the protein sequence. This is called the degeneracy of codons. Therefore, the nucleotide sequence includes nucleotide sequences containing functionally equivalent codons, codons encoding the same amino acids (e.g., arginine or serine are six different codons due to the degeneracy of codons), or codons containing biologically equivalent amino acids.

According to an embodiment of the present disclosure, nucleotide sequences of nucleic acids coding for polypeptides of heavy chain CDRs, light chain CDRs, heavy chain variable regions, light chain variable regions, heavy chains, or light chains in the antibody to CD30 or the antigen-binding fragment thereof according to the present disclosure are listed in the sequence listing appended.

The nucleic acid molecule of the present disclosure which encodes the anti-CD30 antibody or the antigen-binding fragment thereof is construed to encompass nucleotide sequences having substantial identity to the nucleic acid molecule. In this context, the term “substantial identity” refers to an identity of at least 80%, more preferably at least 90%, and most preferably at least 95% between the nucleotide sequence of the present disclosure and any other sequences as analyzed by commonly used algorithm when alignment therebetween is made as much correspondingly as possible.

Considering the above-described mutations having biologically equivalent activity, it should be construed that nucleic acid molecules encoding the antibody or the antigen-binding fragment; or the chimeric antigen receptor polypeptide according to the present disclosure also include sequences having substantial identity therewith. In this regard, the substantial identity refers to an identity of at least 61%, more preferably at least 70%, still more preferably 80%, and most preferably at least 90% between the nucleotide sequence of the present disclosure and any other sequences as analyzed by commonly used algorithm when alignment therebetween is made as much correspondingly as possible. Methods of alignment for sequence comparison are known in the art. Various methods and algorithms for alignment are disclosed in Smith and Waterman, Adv. Appl. Math. 2:482 (1981); Needleman and Wunsch, J. Mol. Bio. 48:443 (1970); Pearson and Lipman, Methods in Mol. Biol. 24:307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992) and Pearson et al., Meth. Mol. Biol. 24:307-31 (1994). The NCBI Basic Local Alignment Search Tool (BLAST; Altschul, et al., J. Mol. Biol. 215:403-10 (1990)) is available from, for example, the NBCI (National Center for Biological Information), and can be used in connection with sequence analysis programs, such as blastp, blasm, blastx, tblastn and tblastx, on the Internet. The use of the program in comparing sequence similarity can be available on the BLAST help page at the NCBI website. The BLAST is available from, for example, the BLAST page of the NBCI website on the Internet. The use of the program in comparing sequence similarity can be available on the BLAST help page at the NCBI website.

In an embodiment of the present disclosure, the nucleic acid molecule comprises nucleotide sequences selected from the group consisting of:
i) the nucleotide sequence of SEQ ID NO: 11, the nucleotide sequence of SEQ ID NO: 12, the nucleotide sequence of SEQ ID NO: 13, the nucleotide sequence of SEQ ID NO: 14, the nucleotide sequence of SEQ ID NO: 15, and the nucleotide sequence of SEQ ID NO: 16, wherein the nucleotide sequence encodes HCDR1, HCDR2, HCDR3, LCDR1, LCDR2, and LCDR3, respectively;
ii) the nucleotide sequence of SEQ ID NO: 31, the nucleotide sequence of SEQ ID NO: 32, the nucleotide sequence of SEQ ID NO: 33, the nucleotide sequence of SEQ ID NO: 34, the nucleotide sequence of SEQ ID NO: 35, and the nucleotide sequence of SEQ ID NO: 36, wherein the nucleotide sequence encodes HCDR1, HCDR2, HCDR3, LCDR1, LCDR2, and LCDR3, respectively;
iii) the nucleotide sequence of SEQ ID NO: 51, the nucleotide sequence of SEQ ID NO: 52, the nucleotide sequence of SEQ ID NO: 53, the nucleotide sequence of SEQ ID NO: 54, the nucleotide sequence of SEQ ID NO: 55, and the nucleotide sequence of SEQ ID NO: 56, wherein the nucleotide sequence encodes HCDR1, HCDR2, HCDR3, LCDR1, LCDR2, and LCDR3, respectively;
iv) the nucleotide sequence of SEQ ID NO: 71, the nucleotide sequence of SEQ ID NO: 72, the nucleotide sequence of SEQ ID NO: 73, the nucleotide sequence of SEQ ID NO: 74, the nucleotide sequence of SEQ ID NO: 75, and the nucleotide sequence of SEQ ID NO: 76, wherein the nucleotide sequence encodes HCDR1, HCDR2, HCDR3, LCDR1, LCDR2, and LCDR3, respectively;
v) the nucleotide sequence of SEQ ID NO: 91, the nucleotide sequence of SEQ ID NO: 92, the nucleotide sequence of SEQ ID NO: 93, the nucleotide sequence of SEQ ID NO: 94, the nucleotide sequence of SEQ ID NO: 95, and the nucleotide sequence of SEQ ID NO: 96, wherein the nucleotide sequence encodes HCDR1, HCDR2, HCDR3, LCDR1, LCDR2, and LCDR3, respectively;
vi) the nucleotide sequence of SEQ ID NO: 111, the nucleotide sequence of SEQ ID NO: 112, the nucleotide sequence of SEQ ID NO: 113, the nucleotide sequence of SEQ ID NO: 114, the nucleotide sequence of SEQ ID NO: 115, and the nucleotide sequence of SEQ ID NO: 116, wherein the nucleotide sequence encodes HCDR1, HCDR2, HCDR3, LCDR1, LCDR2, and LCDR3, respectively;
vii) the nucleotide sequence of SEQ ID NO: 131, the nucleotide sequence of SEQ ID NO: 132, the nucleotide sequence of SEQ ID NO: 133, the nucleotide sequence of SEQ ID NO: 134, the nucleotide sequence of SEQ ID NO: 135, and the nucleotide sequence of SEQ ID NO: 136, wherein the nucleotide sequence encodes HCDR1, HCDR2, HCDR3, LCDR1, LCDR2, and LCDR3, respectively;
viii) the nucleotide sequence of SEQ ID NO: 151, the nucleotide sequence of SEQ ID NO: 152, the nucleotide sequence of SEQ ID NO: 153, the nucleotide sequence of SEQ ID NO: 154, the nucleotide sequence of SEQ ID NO: 155, and the nucleotide sequence of SEQ ID NO: 156, wherein the nucleotide sequence encodes HCDR1, HCDR2, HCDR3, LCDR1, LCDR2, and LCDR3, respectively;
ix) the nucleotide sequence of SEQ ID NO: 171, the nucleotide sequence of SEQ ID NO: 172, the nucleotide sequence of SEQ ID NO: 173, the nucleotide sequence of SEQ ID NO: 174, the nucleotide sequence of SEQ ID NO: 175, and the nucleotide sequence of SEQ ID NO: 176, wherein the nucleotide sequence encodes HCDR1, HCDR2, HCDR3, LCDR1, LCDR2, and LCDR3, respectively;
x) the nucleotide sequence of SEQ ID NO: 191, the nucleotide sequence of SEQ ID NO: 192, the nucleotide sequence of SEQ ID NO: 193, the nucleotide sequence of SEQ ID NO: 194, the nucleotide sequence of SEQ ID NO: 195, and the nucleotide sequence of SEQ ID NO: 196, wherein the nucleotide sequence encodes HCDR1, HCDR2, HCDR3, LCDR1, LCDR2, and LCDR3, respectively; and
xi) the nucleotide sequence of SEQ ID NO: 211, the nucleotide sequence of SEQ ID NO: 212, the nucleotide sequence of SEQ ID NO: 213, the nucleotide sequence of SEQ ID NO: 214, the nucleotide sequence of SEQ ID NO: 215, and the nucleotide sequence of SEQ ID NO: 216, wherein the nucleotide sequence encodes HCDR1, HCDR2, HCDR3, LCDR1, LCDR2, and LCDR3, respectively.

In an embodiment of the present disclosure, the nucleic acid molecule comprises nucleotide sequences selected from the group consisting of:
i) the nucleotide sequence of SEQ ID NO: 17 and the nucleotide sequence of SEQ ID NO: 18, wherein the nucleotide sequence encodes heavy chain variable domain and light chain variable domain, respectively;
ii) the nucleotide sequence of SEQ ID NO: 37 and the nucleotide sequence of SEQ ID NO: 38, wherein the nucleotide sequence encodes heavy chain variable domain and light chain variable domain, respectively;
iii) the nucleotide sequence of SEQ ID NO: 57 and the nucleotide sequence of SEQ ID NO: 58, wherein the nucleotide sequence encodes heavy chain variable domain and light chain variable domain, respectively;
iv) the nucleotide sequence of SEQ ID NO: 77 and the nucleotide sequence of SEQ ID NO: 78, wherein the nucleotide sequence encodes heavy chain variable domain and light chain variable domain, respectively;
v) the nucleotide sequence of SEQ ID NO: 97 and the nucleotide sequence of SEQ ID NO: 98, wherein the nucleotide sequence encodes heavy chain variable domain and light chain variable domain, respectively;
vi) the nucleotide sequence of SEQ ID NO: 117 and the nucleotide sequence of SEQ ID NO: 118, wherein the nucleotide sequence encodes heavy chain variable domain and light chain variable domain, respectively;
vii) the nucleotide sequence of SEQ ID NO: 137 and the nucleotide sequence of SEQ ID NO: 138, wherein the nucleotide sequence encodes heavy chain variable domain and light chain variable domain, respectively;
viii) the nucleotide sequence of SEQ ID NO: 157 and the nucleotide sequence of SEQ ID NO: 158, wherein the nucleotide sequence encodes heavy chain variable domain and light chain variable domain, respectively;
ix) the nucleotide sequence of SEQ ID NO: 177 and the nucleotide sequence of SEQ ID NO: 178, wherein the nucleotide sequence encodes heavy chain variable domain and light chain variable domain, respectively;
x) the nucleotide sequence of SEQ ID NO: 197 and the nucleotide sequence of SEQ ID NO: 198, wherein the nucleotide sequence encodes heavy chain variable domain and light chain variable domain, respectively; and
xi) the nucleotide sequence of SEQ ID NO: 217 and the nucleotide sequence of SEQ ID NO: 218, wherein the nucleotide sequence encodes heavy chain variable domain and light chain variable domain, respectively.

In an embodiment of the present disclosure, the nucleic acid molecule comprises a nucleotide sequence selected from the group consisting of the nucleotide sequence of SEQ ID NO: 19, the nucleotide sequence of SEQ ID NO: 39, the nucleotide sequence of SEQ ID NO: 59, the nucleotide sequence of SEQ ID NO: 79, the nucleotide sequence of SEQ ID NO: 99, the nucleotide sequence of SEQ ID NO: 119, the nucleotide sequence of SEQ ID NO: 139, the nucleotide sequence of SEQ ID NO: 159, the nucleotide sequence of SEQ ID NO: 179, the nucleotide sequence of SEQ ID NO: 199, and the nucleotide sequence of SEQ ID NO: 219, wherein the nucleotide sequence encodes scFv.

Another aspect of the present disclosure provides a recombinant vector carrying the nucleic acid molecule.

According to another aspect thereof, the present disclosure provides a host cell transformed with the recombinant vector.

So long as it allows a vector to be cloned thereto and expressed sequentially, any host cell can be used in the present disclosure. Such host cells are well known in the art. For example, eukaryotic host cells suitable for the vector include monkey kidney cell 7 (COS7), NSO cells, SP2/0, Chinese hamster ovary (CHO) cells, W138, baby hamster kidney (BHK) cells, MDCK, myeloma cell lines, HuT 78 cells, and HEK-293 cells, but are not limited thereto.

According to another aspect thereof, the present disclosure provides a CD30-specific chimeric antigen receptor comprising an anti-CD30 antibody or an antigen-binding fragment thereof; a transmembrane domain; and an intracellular signaling domain.

The anti-CD30 antibody or an antigen binding fragment thereof comprises an immunoglobulin heavy-chain variable region domain and an immunoglobulin light-chain variable region domain, wherein:
i) the heavy-chain variable region domain comprises HCDR1, HCDR2, and HCDR3, wherein the HCDR1, HCDR2, and HCDR3 comprise the amino acid sequence of SEQ ID NO: 1, 2, and 3, respectively; and the light-chain variable region domain comprises LCDR1, LCDR2, and LCDR3, wherein the LCDR1, LCDR2, and LCDR3 comprise the amino acid sequence of SEQ ID NO: 4, 5, and 6, respectively;
ii) the heavy-chain variable region domain comprises HCDR1, HCDR2, and HCDR3, wherein the HCDR1, HCDR2, and HCDR3 comprise the amino acid sequence of SEQ ID NO: 21, 22, and 23, respectively; and the light-chain variable region domain comprises LCDR1, LCDR2, and LCDR3, wherein the LCDR1, LCDR2, and LCDR3 comprise the amino acid sequence of SEQ ID NO: 24, 25, and 26, respectively;
iii) the heavy-chain variable region domain comprises HCDR1, HCDR2, and HCDR3, wherein the HCDR1, HCDR2, and HCDR3 comprise the amino acid sequence of SEQ ID NO: 41, 42, and 43, respectively; and the light-chain variable region domain comprises LCDR1, LCDR2, and LCDR3, wherein the LCDR1, LCDR2, and LCDR3 comprise the amino acid sequence of SEQ ID NO: 44, 45, and 46, respectively;
iv) the heavy-chain variable region domain comprises HCDR1, HCDR2, and HCDR3, wherein the HCDR1, HCDR2, and HCDR3 comprise the amino acid sequence of SEQ ID NO: 61, 62, and 63, respectively; and the light-chain variable region domain comprises LCDR1, LCDR2, and LCDR3, wherein the LCDR1, LCDR2, and LCDR3 comprise the amino acid sequence of SEQ ID NO: 64, 65, and 66, respectively;
v) the heavy-chain variable region domain comprises HCDR1, HCDR2, and HCDR3, wherein the HCDR1, HCDR2, and HCDR3 comprise the amino acid sequence of SEQ ID NO: 81, 82, and 83, respectively; and the light-chain variable region domain comprises LCDR1, LCDR2, and LCDR3, wherein the LCDR1, LCDR2, and LCDR3 comprise the amino acid sequence of SEQ ID NO: 84, 85, and 86, respectively;
vi) the heavy-chain variable region domain comprises HCDR1, HCDR2, and HCDR3, wherein the HCDR1, HCDR2, and HCDR3 comprise the amino acid sequence of SEQ ID NO: 101, 102, and 103, respectively; and the light-chain variable region domain comprises LCDR1, LCDR2, and LCDR3, wherein the LCDR1, LCDR2, and LCDR3 comprise the amino acid sequence of SEQ ID NO: 104, 105, and 106, respectively;
vii) the heavy-chain variable region domain comprises HCDR1, HCDR2, and HCDR3, wherein the HCDR1, HCDR2, and HCDR3 comprise the amino acid sequence of SEQ ID NO: 121, 122, and 123, respectively; and the light-chain variable region domain comprises LCDR1, LCDR2, and LCDR3, wherein the LCDR1, LCDR2, and LCDR3 comprise the amino acid sequence of SEQ ID NO: 124, 125, and 126, respectively;
viii) the heavy-chain variable region domain comprises HCDR1, HCDR2, and HCDR3, wherein the HCDR1, HCDR2, and HCDR3 comprise the amino acid sequence of SEQ ID NO: 141, 142, and 143, respectively; and the light-chain variable region domain comprises LCDR1, LCDR2, and LCDR3, wherein the LCDR1, LCDR2, and LCDR3 comprise the amino acid sequence of SEQ ID NO: 144, 145, and 146, respectively;
ix) the heavy-chain variable region domain comprises HCDR1, HCDR2, and HCDR3, wherein the HCDR1, HCDR2, and HCDR3 comprise the amino acid sequence of SEQ ID NO: 161, 162, and 163, respectively; and the light-chain variable region domain comprises LCDR1, LCDR2, and LCDR3, wherein the LCDR1, LCDR2, and LCDR3 comprise the amino acid sequence of SEQ ID NO: 164, 165, and 166, respectively;
x) the heavy-chain variable region domain comprises HCDR1, HCDR2, and HCDR3, wherein the HCDR1, HCDR2, and HCDR3 comprise the amino acid sequence of SEQ ID NO: 181, 182, and 183, respectively; and the light-chain variable region domain comprises LCDR1, LCDR2, and LCDR3, wherein the LCDR1, LCDR2, and LCDR3 comprise the amino acid sequence of SEQ ID NO: 184, 185, and 186, respectively; or
xi) the heavy-chain variable region domain comprises HCDR1, HCDR2, and HCDR3, wherein the HCDR1, HCDR2, and HCDR3 comprise the amino acid sequence of SEQ ID NO: 201, 202, and 203, respectively; and the light-chain variable region domain comprises LCDR1, LCDR2, and LCDR3, wherein the LCDR1, LCDR2, and LCDR3 comprise the amino acid sequence of SEQ ID NO: 204, 205, and 206, respectively.

In an embodiment of the present disclosure, the anti-CD30 antibody or the antigen binding fragment thereof comprises a heavy chain variable region domain and a light chain variable region domain, wherein:
i) the heavy-chain variable region domain comprises the amino acid sequence of SEQ ID NO: 7; and the light-chain variable region domain comprises the amino acid sequence of SEQ ID NO: 8;
ii) the heavy-chain variable region domain comprises the amino acid sequence of SEQ ID NO: 27; and the light-chain variable region domain comprises the amino acid sequence of SEQ ID NO: 28;
iii) the heavy-chain variable region domain comprises the amino acid sequence of SEQ ID NO: 47; and the light-chain variable region domain comprises the amino acid sequence of SEQ ID NO: 48;
iv) the heavy-chain variable region domain comprises the amino acid sequence of SEQ ID NO: 67; and the light-chain variable region domain comprises the amino acid sequence of SEQ ID NO: 68;
v) the heavy-chain variable region domain comprises the amino acid sequence of SEQ ID NO: 87; and the light-chain variable region domain comprises the amino acid sequence of SEQ ID NO: 88;
vi) the heavy-chain variable region domain comprises the amino acid sequence of SEQ ID NO: 107; and the light-chain variable region domain comprises the amino acid sequence of SEQ ID NO: 108;
vii) the heavy-chain variable region domain comprises the amino acid sequence of SEQ ID NO: 127; and the light-chain variable region domain comprises the amino acid sequence of SEQ ID NO: 128;
viii) the heavy-chain variable region domain comprises the amino acid sequence of SEQ ID NO: 147; and the light-chain variable region domain comprises the amino acid sequence of SEQ ID NO: 148;
ix) the heavy-chain variable region domain comprises the amino acid sequence of SEQ ID NO: 167; and the light-chain variable region domain comprises the amino acid sequence of SEQ ID NO: 168;
x) the heavy-chain variable region domain comprises the amino acid sequence of SEQ ID NO: 187; and the light-chain variable region domain comprises the amino acid sequence of SEQ ID NO: 188; or
xi) the heavy-chain variable region domain comprises the amino acid sequence of SEQ ID NO: 207; and the light-chain variable region domain comprises the amino acid sequence of SEQ ID NO: 208.

As used herein, the term “chimeric antigen receptor (CAR)” refers to an artificially constructed hybrid protein or polypeptide in which an antigen-binding domain (e.g., single-chain variable fragment (scFv)) of an antibody is linked to a T-cell signaling or T-cell activating domain. Taking advantage of the antigen-binding function of a monoclonal antibody, chimeric antigen receptors give T cells the new ability to retarget a specific protein in a non-MHC-restricted manner.

Non-MHC-restricted antigen recognition provides CAR-expressing T cells with an ability to recognize irrespective of antigen processing, thus avoiding main tumor escape mechanisms. In addition, when expressed in T cells, CAR does advantageously not dimerize with intrinsic T-cell receptor (TCR) alpha and beta chains.

The chimeric antigen receptor of the present disclosure comprises an extracellular domain containing an antibody induced against CD30, known as a B lymphocyte antigen, or against an antigen-binding fragment thereof. In the present disclosure, the antibody induced against CD30 or an antigen-binding fragment thereof is as defined above for the anti-CD30 antibody or the antigen-binding fragment thereof.

In an embodiment of the present disclosure, the anti-CD30 antibody or the antigen binding fragment thereof comprises scFv, wherein the scFv comprises an amino acid sequence selected from the group consisting of the amino acid sequence of SEQ ID NO: 9, the amino acid sequence of SEQ ID NO: 29, the amino acid sequence of SEQ ID NO: 49, the amino acid sequence of SEQ ID NO: 69, the amino acid sequence of SEQ ID NO: 89, the amino acid sequence of SEQ ID NO: 109, the amino acid sequence of SEQ ID NO: 129, the amino acid sequence of SEQ ID NO: 149, the amino acid sequence of SEQ ID NO: 169, the amino acid sequence of SEQ ID NO: 189, and the amino acid sequence of SEQ ID NO: 209.

In an embodiment of the present disclosure, the CD30-specific chimeric antigen receptor comprises scFv CAR, wherein the scFv CAR comprises an amino acid sequence selected from the group consisting of the amino acid sequence of SEQ ID NO:10, the amino acid sequence of SEQ ID NO: 30, the amino acid sequence of SEQ ID NO: 50, the amino acid sequence of SEQ ID NO: 70, the amino acid sequence of SEQ ID NO: 90, the amino acid sequence of SEQ ID NO: 110, the amino acid sequence of SEQ ID NO: 130, the amino acid sequence of SEQ ID NO: 150, the amino acid sequence of SEQ ID NO: 170, the amino acid sequence of SEQ ID NO: 190, and the amino acid sequence of SEQ ID NO: 210.

According to an embodiment of the present disclosure, the intracellular signaling domain includes functional signaling of 4-1BB, CD28, OX40, CD3 zeta, or a combination thereof.

According to an embodiment of the present disclosure, the chimeric antigen receptor of the present disclosure is expressed on cell surfaces. Hence, the chimeric antigen receptor may comprise a transmembrane domain. The transmembrane domain may be derived from natural or synthetic sources known in the art. By way of example, the transmembrane domain may be a transmembrane domain of the protein selected from the group consisting of alpha, beta, or zeta chains of T-cell receptor, CD27, CD28, CD3 epsilon, CD45, CD4, CD5, CD8 (CD8α, CD8β, CD8γ), CD9, CD16, CD22, CD33, CD37, CD64, CD80, CD86, CD134, CD137, and CD154 (CD154α, CD154β, CD154ζ), but is not limited thereto.

The term “intracellular signaling domain”, as used herein, refers to a functional protein domain that produces a 2^{nd} messenger or functions as an effector in response to the 2^{nd} messenger to intracellularly transfer information so as to regulate cellular activity via a defined signaling pathway.

According to another embodiment of the present disclosure, the chimeric antigen receptor of the present disclosure may comprise an intracellular signaling domain. The intracellular signaling domain is responsible for intracellular signaling following the binding of extracellular ligand binding domain to the target (e.g., CD30) resulting in the activation of the immune cell and immune response. In other words, the signal transducing domain is responsible for the activation of at least one of the normal effector functions of the immune cell in which the chimeric antigen receptor is expressed. For example, the effector function of a T cell can be a cytolytic activity or helper activity including the secretion of cytokines. Preferred examples of signal transducing domain for use in a chimeric antigen receptor can be the cytoplasmic sequences of the T cell receptor and co-receptors that act in concert to initiate signal transduction following antigen receptor engagement, as well as any derivate or variant of these sequences and any synthetic sequence that has the same functional capability.

According to an exemplary embodiment, the intracellular signaling domain of the chimeric antigen receptor is a domain derived from CD3ζ (CD3 zeta) chain.

According to another exemplary embodiment of the present disclosure, the intracellular signaling domain of chimeric antigen receptor further comprises at least one intracellular (cytoplasmic) region of a costimulatory molecule selected from the group consisting of OX40 (CD134), CD2, CD27, CD28, CDS, ICAM-1, LFA-1 (CD11a/CD18), and 4-1BB (CD137). The intracellular signaling domain may be obtained or derived from an intracellular signaling molecule and may include an entirety or a part of the molecule from which the intracellular signaling domain is derived, as well as the domain described above.

According to an exemplary embodiment of the present disclosure, the costimulatory molecule (domain) comprises a functional signaling domain obtained from a protein selected from the group consisting of CD28, OX40, 4-1BB (CD137), and/or ICOS (CD278) and, more particularly, a functional signaling domain of CD28 and/or OX40.

According to another embodiment of the present disclosure, the intracellular signaling domain comprises a functional signaling domain of 4-1BB, CD28, OX40, CD3 zeta, or a combination thereof. Most particularly, the intracellular signaling domain is a functional signaling domain of CD3 zeta.

The transmembrane domain and intracellular signaling domain in the chimeric antigen receptor of the present disclosure may be at least one combination selected from among the transmembrane domains and intracellular signaling domains described above. For example, the chimeric antigen receptor of the present disclosure may comprise the CD8a transmembrane domain and the intracellular signaling domains of CD28 and CD3ζ.

Another aspect of the present disclosure provides a nucleic acid molecule encoding the chimeric antigen receptor described above.

The above-mentioned anti-CD30 antibody or the antigen-binding fragment (polypeptide) thereof, the nucleic acid molecule coding therefor, the chimeric antigen receptor comprising the anti-CD30 antibody or the antigen-binding fragment thereof, and the nucleic acid molecule coding for the chimeric antigen receptor are each in an isolated state.

As used herein, the term “isolated” means altered or removed from the natural/native state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

According to another aspect thereof, the present disclosure provides a recombinant vector carrying the above-mentioned nucleic acid molecule. For the “vector” to be described hereinafter, the antibody or the antigen-binding fragment thereof, or the nucleic acid molecule encoding a chimeric antigen receptor are commonly applied.

The term “vector” is intended to encompass a transfer vector and an expression vector.

As used herein, the term “transfer vector” refers to a composition of matter which comprises an isolated nucleic acid and which can be used to deliver the isolated nucleic acid into the interior of a cell. Examples of the transfer vector include, but are not limited to, linear polynucleotides, polynucleotides associated with ionic or amphiphilic compounds, plasmids, and viruses. More particularly, the transfer vector includes an autonomously replicating plasmid or virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector”, as used herein, refers to a vector comprising a recombinant polynucleotide comprising expression control sequences operatively linked to a nucleotide sequence to be expressed in a host cell. An expression vector comprises sufficient cis-acting elements for expression; other elements for expression can be supplied by the host cell or in an in vitro expression system. Expression vectors include plasmids; cosmids; and viruses, such as bacteriophages, adenoviruses, lentiviruses, retroviruses, and adeno-associated viruses, which all incorporate the recombinant polynucleotide. According to an exemplary embodiment of the present disclosure, a nucleic acid molecule coding for the antibody or antigen-binding fragment, or the chimeric antigen receptor is operatively linked to a promoter in the vector of the present disclosure. As used herein, the term “operatively linked” means a functional linkage between a regulatory sequence for nucleic acid expression (example: a promoter, a signal sequence, or array of positions to which transcriptional factors bind) and other nucleic acid sequences, and by which the regulatory sequences are able to control the transcription and/or translation of the other nucleic acid sequence.

The recombinant vector system of the present disclosure can be constructed using various methods known in the art. With respect to concrete methods, reference may be made to Sambrook et al., Molecular Cloning, A Laboratory Manual, Cold Spring Harbor Laboratory Press (2001), which is incorporated herein by reference.

The vector of the present disclosure may be constructed as a vector for gene cloning, for protein expression, or for gene transfer. Also, the vectors of the present disclosure may be constructed for eukaryotic or prokaryotic cells serving as host cells.

For example, when the present vector is an expression vector in a eukaryotic cell, promoters derived from genomes of mammalian cells (e.g., a metallothionein promoter, β-actin promoter, human hemoglobin promoter and human muscle creatinine promoter) or promoters derived from mammalian viruses (e.g., an adenovirus late promoter, a vaccinia virus 7.5K promoter, SV40 promoter, cytomegalovirus promoter, a tk promoter of HSV, a promoter of mouse mammary tumor virus (MMTV), a LTR promoter of HIV, a promoter of moloney virus, a promoter of Epstein Barr Virus (EBV), a promoter of Rous Sarcoma Virus (RSV)) may be use. Generally, the vectors include a polyadenylate sequence as a transcriptional termination sequence.

According to an embodiment of the present disclosure, when used as a transfer vector, the vector may be “retroviral vector”. Retroviruses provide a convenient platform for gene delivery systems. A selected gene can be inserted into a vector and packaged in retroviral particles. The recombinant virus can then be delivered to cells of the subject either in vivo or in vitro. A number of retroviral systems are known in the art. In some exemplary embodiments, the retroviral vector may be a pMT retroviral vector, which is an MLV-based retroviral vector, but is not limited thereto.

According to an embodiment of the present disclosure, the vector may be a lentivirus vector or an adenovirus vector.

The recombinant vector of the present disclosure may be fused with additional nucleotide sequences to facilitate the isolation and purification of the polypeptide expressed from the vector. The nucleotide sequences to be fused with the present vector include, for example, glutathione S-transferase (Pharmacia, USA), maltose binding protein (NEB, USA), FLAG (IBI, USA) and 6×His (hexahistidine; Qiagen, USA) and the like. The expression vector of the present disclosure may also comprise a selectable marker gene and/or a reporter gene as a selection marker for evaluating the expression of the antibody or the antigen-binding fragment and the CAR polypeptide containing the antibody. The selectable marker gene may be an antibiotic resistant gene typically used in the art, examples of which include genes resistant to ampicillin, gentamicin, carbenicillin, chloramphenicol, streptomycin, kanamycin, geneticin, neomycin, and tetracycline. The reporter gene may be exemplified by luciferase, beta-galactosidase, chloramphenicol acetyl transferase, and green fluorescent protein genes.

Methods of introducing and expressing genes into a cell are known in the art. In the context of an expression vector, the vector can be readily introduced into a host cell, e.g., mammalian, bacterial, yeast, or insect cell by any method known in the art. For example, the expression vector can be transferred into a host cell by physical, chemical, or biological means. The physical means include calcium phosphate precipitation, lipofection, particle bombardment, microinjection, electroporation, and the like. The chemical means include colloidal dispersion systems, such as macromolecule complexes, nanocapsules, microspheres, beads, and lipid-based systems including oil-in-water emulsions, micelles, mixed micelles, and liposomes. Within the biological means are the use of DNA or RNA vectors such as lentivirus, retrovirus, and the like.

According to an aspect thereof, the present disclosure provides an effector cell expressing the chimeric antigen receptor.

In one embodiment of the present disclosure, the effector cell refers to a cell of hematopoietic origin functionally involved in the initiation and/or execution of an innate and/or adaptive immune response.

The immune cell according to the present disclosure may be derived from a stem cell. The stem cells may be adult stem cell, non-human embryonic stem cells, cord blood stem cells, bone marrow stem cells, induced pluripotent stem cells, or hematopoietic stem cells. More particularly, the immune cells may be selected from the group consisting of dendritic cells, killer dendritic cells, mast cells, NK-cells, B-cells or inflammatory T-lymphocytes, cytotoxic T-lymphocytes, regulatory T-lymphocytes, and helper T-lymphocytes, but are not limited thereto.

The effector cells include a population of autologous or allogeneic cells. In other words, the effector cells include a population of autologous or allogeneic cells expressing CAR specific for CD30.

According to an embodiment of the present disclosure, the effector cells include a population of cells transduced or transfected with a vector carrying a nucleic acid molecular coding for a CD30-specific CAR. The transfection or transduction can be achieved by various means known in the art as described above, without limitations.

Hence, according to an exemplary embodiment of the present disclosure, after being delivered into the effector cells, e.g., T lymphocytes or natural killer cells, the nucleic acid molecule coding for the CD30-specific CAR is transcribed into mRNA from which a CD30-specific CAR polypeptide is then translated, and expressed on the cell surface.

Also, another aspect of the present disclosure provides a pharmaceutical composition comprising the anti-CD30 antibody of the present disclosure or an antigen-binding fragment thereof or a pharmaceutical composition comprising a cell expressing the chimeric antigen receptor of the present disclosure.

The pharmaceutical composition may be provided in the form of a pharmaceutical composition comprising: the anti-CD30 antibody of the present disclosure or an antigen-binding fragment thereof or the chimeric antigen receptor-expressing cell; and a pharmaceutically acceptable carrier.

When administered in the form of a pharmaceutical composition, the cell expressing the chimeric antigen receptor of the present disclosure may be a cell derived from an animal allogenic to the subject, or a cell autologous cell.

The pharmaceutical composition of the present disclosure may comprise a population of cells expressing the chimeric antigen receptor of the present disclosure.

The pharmaceutical composition of the present disclosure comprises the anti-CD30 antibody of the present disclosure or an antigen-binding fragment thereof or the chimeric antigen receptor-expressing cell as an effective ingredient. Accordingly, the common descriptions between them are omitted in order to avoid undue redundancy leading to the complexity of this specification.

As will be proven in the following Example, when the chimeric antigen receptor T cells containing the CD30 antibody fragment of the present disclosure and a CD30 antigen-expressing cell line are co-cultured, the CD30 antigen on the surface of the CD30-positive cell line is recognized to induce the activation of the chimeric antigen receptor. Thus, the pharmaceutical composition of the present disclosure is expected to find advantageous applications in the treatment of CD30 antigen-related diseases.

The pharmaceutically acceptable carrier contained in the pharmaceutical composition of the present disclosure is ordinarily used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further comprise a lubricant, a wetting agent, a sweetening agent, a flavoring agent, an emulsifier, a suspending agent, a preservative, and the like in addition to the above ingredients. Suitable pharmaceutically acceptable carriers and preparations are described in detail in Remington's Pharmaceutical Sciences (19th ed., 1995).

The pharmaceutical composition of the present disclosure may be administered orally or parenterally, for example, by intravenous injection, subcutaneous injection, intramuscular injection, intraperitoneal injection, intrasternal injection, intratumoral injection, topical administration, intranasal administration, intrapulmonary administration, and rectal administration.

The appropriate dose of the pharmaceutical composition of the present disclosure varies depending on factors, such as a formulating method, a manner of administration, patient's age, body weight, gender, and morbidity, food, a time of administration, a route of administration, an excretion rate, and response sensitivity. An ordinarily skilled practitioner can easily determine and prescribe an effective dose for desired treatment or prevention. According to a preferable embodiment of the present disclosure, the daily dose of the pharmaceutical composition of the present disclosure is 0.0001-100 mg/kg. As used herein, the term “pharmaceutically effective amount” refers to an amount sufficient to prevent or treat the above-described diseases.

As used herein, the term “prevention” refers to a prophylactic or protective treatment of a disease or a disease condition. As used herein, the term “treatment” refers to a reduction, suppression, relief, or eradication of a disease condition.

The pharmaceutical composition of the present disclosure may be formulated into a unit dosage form or may be prepared in a multi-dose container by using a pharmaceutically acceptable carrier and/or excipient according to a method that can be easily implemented by a person having an ordinary skill in the art to which the present disclosure pertains. Here, the formulation may be in the form of a solution in an oily or aqueous medium, a suspension, an emulsion, an extract, a pulvis, a suppository, a powder, a granule, a tablet, or a capsule, and may further contain a dispersant or a stabilizer.

The pharmaceutical composition of the present disclosure may further comprise other pharmaceutically active agents or drugs, for example, chemotherapeutic agents such as asparaginase, busulfane, carboplatin, cisplatin, daunorubicin, doxorubicin, fluorouracil, gemcitabine, hydroxyurea, methotrexate, paclitaxel, rituximab, vinblastine, vincristine, and the like; targeted therapeutic agents such as bevacizumab, olaparib, and the like; or immune checkpoint inhibitors such as nivolumab, pembrolizumab, and the like, in addition to the above-described chimeric antigen receptor-expressing cells, or may be administered in combination therewith.

According to another aspect thereof, the present disclosure provides a method for prevention or treatment of a CD30-expressing cell-associated disease, the method comprising a step of administering to a subject in need thereof a composition comprising an anti-CD30 antibody or an antigen-binding fragment thereof; or a composition comprising a cell expressing the chimeric antigen receptor.

Accordingly, human antibodies of the present invention can be used to treat and/or prevent a variety of CD30 mediated diseases by administering the anti-CD30 antibody or the effector cell harboring the chimeric antigen receptor to patients suffering from various CD30-mediated diseases. Exemplary diseases that can be treated (e.g., ameliorated) or prevented include, but are not limited to, tumorigenic diseases and autoimmune diseases. Examples of tumorigenic diseases which can be treated and/or prevented include B cell malignancy selected from the group consisting of chronic lymphocytic leukemia (CLL), acute lymphocytic leukemia (ALL), pro-lymphocytic leukemia, hairy cell leukemia, common acute lymphocytic leukemia (CALLA), null-acute lymphoblastic leukemia, non-Hodgkin's lymphoma, diffuse large B cell lymphoma (DLBCL), anaplastic large-cell lymphoma, multiple myeloma, follicular lymphoma, splenic lymphoma, marginal zone lymphoma, mantle cell lymphoma, indolent B cell lymphoma, and Hodgkin lymphoma, Hodgkin's disease, anaplastic large cell lymphoma (ALCL), adult T-cell lymphoma (ATL), angioimmunoblastic lymphadenopathy (AILD)-like T cell lymphoma, peripheral T-cell lymphoma, cutaneous T-cell lymphoma (CTCL), NK/T cell lymphoma, HIV associated body cavity-based lymphomas, embryonal carcinomas, undifferentiated carcinomas of the rhino-pharynx (e.g., Schmincke's tumor), Castleman's disease, Kaposi's Sarcoma and other T-cell or B-cell lymphomas. Germ cell tumor may also be included. Examples of autoimmune diseases which can be treated and/or prevented include rheumatoid arthritis, systemic lupus erythematosus, systemic sclerosis, atopic dermatitis, Graves' disease, Hashimoto's thyroiditis, Wegner's granulomatosis, Omen's syndrome, chronic renal failure, acute infectious mononucleosis, HIV and herpes virus associated diseases.

In an embodiment of the present invention, the subject is a mammalian animal or a human.

Since the method for the prevention or treatment of cancer or inflammatory disease according to the present disclosure employs the above-described antibody or antigen-binding fragment; or the chimeric antigen receptor-expressing effector cell as an effective ingredient, the overlapping descriptions thereof are omitted to avoid undue complexity of the specification.

Features and advantages of the present disclosure are summarized as follows:
(a) The present disclosure provides an anti-CD30 antibody or an antigen-binding fragment thereof.
(b) The present disclosure provides a nucleic acid molecule encoding an anti-CD30 antibody or an antigen-binding fragment thereof.
(c) The present disclosure provides a CD30-specific chimeric antigen receptor comprising: an anti-CD30 antibody or an antigen-binding fragment thereof; a transmembrane domain; and intracellular signaling domain.
(d) The antibody of the present disclosure binds specifically to CD30 that is highly expressed in cancer cells (particularly, blood cancer) and has very low CDR sequence homology to conventional CD30 target antibodies. Thus, the antibody of the present disclosure has a characteristic sequence. Inducing immune cell activation in response to stimulation with CD30-positive cells, cells expressing the chimeric antigen receptor containing the anti-CD30 antibody or the antigen-binding fragment (e.g., scFv) of the present disclosure can be advantageously used as a CAR-immune cell therapy product.

Information about the amino acid and nucleotide sequences used in this specification is provided below.

| No. | Type | Name | Sequence |
|---|---|---|---|
| 1 | Protein | CDRH1 of anti CD30 | GYGMS |
| | | antibody (T1-2) | |
| 2 | Protein | CDRH2 of anti CD30 | YISGYSYYTYYADSVKG |
| | | antibody (T1-2) | |
| 3 | Protein | CDRH3 of anti CD30 | YDGNGFDY |
| | | antibody (T1-2) | |
| 4 | Protein | CDRL1 of anti CD30 | SGSSSNIGSNYVY |
| | | antibody (T1-2) | |
| 5 | Protein | CDRL2 of anti CD30 | RNNQRPS |
| | | antibody (T1-2) | |
| 6 | Protein | CDRL3 of anti CD30 | AADSSDDS |
| | | antibody (T1-2) | |
| 7 | Protein | Heavy chain variable | EVQLLESGGGLVQPGGSLRLSCAASGFTFSGYGMSWVR |
| | | domain of anti CD30 | QAPGKGLEWVSYISGYSYYTYYADSVKGRFTFSRDNSKN |
| | | antibody (T1-2) | TLYLQMNSLRAEDTAVYYCARYDGNGFDYWGQGTLVTV |
| | | | SS |
| 8 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAADSSDDSYVFGGGTKLTVLG |
| | | (T1-2) | |
| 9 | Protein | scFv of anti CD30 | EVQLLESGGGLVQPGGSLRLSCAASGFTFSGYGMSWVR |
| | | antibody | QAPGKGLEWVSYISGYSYYTYYADSVKGRFTFSRDNSKN |
| | | (T1-2) | TLYLQMNSLRAEDTAVYYCARYDGNGFDYWGQGTLVTV |
| | | | SSGGGGSGGGGSGGGGSQSVLTQPPSASGTPGQRVTI |
| | | | SCSGSSSNIGSNYVYWYQQLPGTAPKLLIYRNNQRPSGV |
| | | | PDRFSGSKSGTSASLAISGLRSEDEADYYCAADSSDDSY |
| | | | VFGGGTKLTVLG |
| 10 | Protein | scFv CAR of anti | EVQLLESGGGLVQPGGSLRLSCAASGFTFSGYGMSWVR |
| | | CD30 antibody | QAPGKGLEWVSYISGYSYYTYYADSVKGRFTFSRDNSKN |
| | | (T1-2) | TLYLQMNSLRAEDTAVYYCARYDGNGFDYWGQGTLVTV |
| | | | SSGGGGSGGGGSGGGGSQSVLTQPPSASGTPGQRVTI |
| | | | SCSGSSSNIGSNYVYWYQQLPGTAPKLLIYRNNQRPSGV |
| | | | PDRFSGSKSGTSASLAISGLRSEDEADYYCAADSSDDSY |
| | | | VFGGGTKLTVLGGQAGQTTTPAPRPPTPAPTIASQPLSLR |
| | | | PEACRPAAGGAVHTRGLDFACDIYIWAPLAGTCGVLLLSL |
| | | | VITLYCKRGRKKLLYIFKQPFMRPVQTTQEEDGCSCRFPE |
| | | | EEEGGCELRVKFSRSADAPAYQQGQNQLYNELNLGRRE |
| | | | EYDVLDKRRGRDPEMGGKPRRKNPQEGLYNELQKDKM |
| | | | AEAYSEIGMKGERRRGKGHDGLYQGLSTATKDTYDALH |
| | | | MQALPPR |
| 11 | DNA | Nucleotide sequence | GGTTATGGTATGAGC |
| | | for encoding CDRH1 | |
| | | of anti CD30 antibody | |
| | | (T1-2) | |
| 12 | DNA | Nucleotide sequence | TACATCTCTGGTTACTCTTACTACACGTATTACGCTGAT |
| | | for encoding CDRH2 | TCTGTAAAAGGT |
| | | of anti CD30 antibody | |
| | | (T1-2) | |
| 13 | DNA | Nucleotide sequence | TACGACGGTAACGGTTTCGACTAC |
| | | for encoding CDRH3 | |
| | | of anti CD30 antibody | |
| | | (T1-2) | |
| 14 | DNA | Nucleotide sequence | AGTGGCTCTTCATCTAATATTGGCTCTAATTATGTCTAC |
| | | for encoding CDRL1 of | |
| | | anti CD30 antibody | |
| | | (T1-2) | |
| 15 | DNA | Nucleotide sequence | AGAAATAACCAGCGGCCAAGC |
| | | for encoding CDRL2 of | |
| | | anti CD30 antibody | |
| | | (T1-2) | |
| 16 | DNA | Nucleotide sequence | GCTGCTGACTCTTCTGACGACTCT |
| | | for encoding CDRL3 of | |
| | | anti CD30 antibody | |
| | | (T1-2) | |
| 17 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCGGTTATGGTATGAGCTGGGTCC |
| | | of anti CD30 antibody | GCCAGGCTCCAGGGAAGGGGCTGGAGTGGGTCTCAT |
| | | (T1-2) | ACATCTCTGGTTACTCTTACTACACGTATTACGCTGATT |
| | | | CTGTAAAAGGTCGGTTCACCTTCTCCAGAGACAATTCC |
| | | | AAGAACACGCTGTATCTGCAAATGAACAGCCTGAGAGC |
| | | | CGAGGACACGGCCGTGTATTACTGTGCGCGTTACGAC |
| | | | GGTAACGGTTTCGACTACTGGGGCCAGGGTACACTGG |
| | | | TCACCGTGAGCTCA |
| 18 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 antibody | GCAGCTCCCAGGAACGGCCCCCAAACTCCTCATCTAT |
| | | (T1-2) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACTCTTCTGACGACTCTTATGTCTTC |
| | | | GGCGGAGGCACCAAGCTGACGGTCCTAGGT |
| 19 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCGGTTATGGTATGAGCTGGGTCC |
| | | (T1-2) | GCCAGGCTCCAGGGAAGGGGCTGGAGTGGGTCTCAT |
| | | | ACATCTCTGGTTACTCTTACTACACGTATTACGCTGATT |
| | | | CTGTAAAAGGTCGGTTCACCTTCTCCAGAGACAATTCC |
| | | | AAGAACACGCTGTATCTGCAAATGAACAGCCTGAGAGC |
| | | | CGAGGACACGGCCGTGTATTACTGTGCGCGTTACGAC |
| | | | GGTAACGGTTTCGACTACTGGGGCCAGGGTACACTGG |
| | | | TCACCGTGAGCTCAGGTGGAGGCGGTTCAGGCGGAG |
| | | | GTGGATCCGGCGGTGGCGGATCGCAGTCTGTGCTGAC |
| | | | TCAGCCACCCTCAGCGTCTGGGACCCCCGGGCAGAG |
| | | | GGTCACCATCTCTTGTAGTGGCTCTTCATCTAATATTG |
| | | | GCTCTAATTATGTCTACTGGTACCAGCAGCTCCCAGGA |
| | | | ACGGCCCCCAAACTCCTCATCTATAGAAATAACCAGCG |
| | | | GCCAAGCGGGGTCCCTGACCGATTCTCTGGCTCCAAG |
| | | | TCTGGCACCTCAGCCTCCCTGGCCATCAGTGGGCTCC |
| | | | GGTCCGAGGATGAGGCCGATTATTACTGTGCTGCTGA |
| | | | CTCTTCTGACGACTCTTATGTCTTCGGCGGAGGCACCA |
| | | | AGCTGACGGTCCTAGGT |
| 20 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCGGTTATGGTATGAGCTGGGTCC |
| | | antibody | GCCAGGCTCCAGGGAAGGGGCTGGAGTGGGTCTCAT |
| | | (T1-2) | ACATCTCTGGTTACTCTTACTACACGTATTACGCTGATT |
| | | | CTGTAAAAGGTCGGTTCACCTTCTCCAGAGACAATTCC |
| | | | AAGAACACGCTGTATCTGCAAATGAACAGCCTGAGAGC |
| | | | CGAGGACACGGCCGTGTATTACTGTGCGCGTTACGAC |
| | | | GGTAACGGTTTCGACTACTGGGGCCAGGGTACACTGG |
| | | | TCACCGTGAGCTCAGGTGGAGGCGGTTCAGGCGGAG |
| | | | GTGGATCCGGCGGTGGCGGATCGCAGTCTGTGCTGAC |
| | | | TCAGCCACCCTCAGCGTCTGGGACCCCCGGGCAGAG |
| | | | GGTCACCATCTCTTGTAGTGGCTCTTCATCTAATATTG |
| | | | GCTCTAATTATGTCTACTGGTACCAGCAGCTCCCAGGA |
| | | | ACGGCCCCCAAACTCCTCATCTATAGAAATAACCAGCG |
| | | | GCCAAGCGGGGTCCCTGACCGATTCTCTGGCTCCAAG |
| | | | TCTGGCACCTCAGCCTCCCTGGCCATCAGTGGGCTCC |
| | | | GGTCCGAGGATGAGGCCGATTATTACTGTGCTGCTGA |
| | | | CTCTTCTGACGACTCTTATGTCTTCGGCGGAGGCACCA |
| | | | AGCTGACGGTCCTAGGTGGCCAGGCCGGCCAGACAA |
| | | | CGACACCTGCTCCCAGACCGCCTACTCCCGCCCCAAC |
| | | | CATTGCATCTCAGCCACTCTCTCTGAGACCCGAAGCGT |
| | | | GTAGACCTGCGGCCGGGGGCGCTGTCCACACAAGAG |
| | | | GCTTAGACTTCGCCTGCGATATCTATATCTGGGCCCCA |
| | | | CTCGCAGGCACTTGTGGAGTGCTGCTGCTTTCACTCGT |
| | | | GATAACCCTGTACTGCAAAAGGGGGAGAAAGAAGCTG |
| | | | CTGTATATTTTTAAACAACCATTTATGAGACCTGTTCAG |
| | | | ACTACCCAGGAAGAAGACGGTTGTAGTTGCAGATTCCC |
| | | | CGAGGAGGAAGAAGGAGGTTGCGAGTTGAGAGTAAAG |
| | | | TTCAGCAGATCCGCAGATGCCCCTGCTTACCAGCAGG |
| | | | GTCAAAACCAGCTTTACAACGAGCTGAATTTAGGTAGA |
| | | | AGAGAGGAATATGACGTGTTGGATAAAAGAAGAGGAA |
| | | | GAGACCCGGAAATGGGCGGCAAGCCTCGAAGAAAAAA |
| | | | TCCCCAAGAGGGACTCTACAATGAGCTGCAGAAGGAC |
| | | | AAAATGGCTGAAGCCTACAGCGAGATCGGCATGAAGG |
| | | | GAGAAAGACGCAGAGGGAAAGGGCATGATGGGCTTTA |
| | | | TCAGGGCTTGTCCACCGCTACAAAGGATACTTATGACG |
| | | | CACTACACATGCAGGCCCTGCCACCCCGT |
| 21 | Protein | CDRH1 of anti CD30 | GYGMS |
| | | antibody (T1-7) | |
| 22 | Protein | CDRH2 of anti CD30 | YISSGSYYTYYADSVKG |
| | | antibody (T1-7) | |
| 23 | Protein | CDRH3 of anti CD30 | YRGDNDYYGYFDY |
| | | antibody (T1-7) | |
| 24 | Protein | CDRL1 of anti CD30 | SCSSSNIGNNAVS |
| | | antibody (T1-7) | |
| 25 | Protein | CDRL2 of anti CD30 | RNNQRPS |
| | | antibody (T1-7) | |
| 26 | Protein | CDRL3 of anti CD30 | AADYGSD |
| | | antibody (T1-7) | |
| 27 | Protein | Heavy chain variable | EVQLLESGGGLVQPGGSLRLSCAASGFTFSGYGMSWVR |
| | | domain of anti CD30 | QAPGKGLEWVSYISSGSYYTYYADSVKGRFTISRDNSKN |
| | | antibody (T1-7) | TLYLQMNSLRAEDTAVYYCARYRGDNDYYGYFDYWGQG |
| | | | TLVTVSS |
| 28 | Protein | Light chain variable | QSVLTQPPSASGTPGQRVTISCSCSSSNIGNNAVSWYQQ |
| | | domain of anti CD30 | LPGTAPKLLIYRNNQRPSGVPDRFSGSKSGTSASLAISGL |
| | | antibody (T1-7) | RSEDEADYYCAADYGSDYVFGGGTKLTVLG |
| 29 | Protein | scFv of anti CD30 | EVQLLESGGGLVQPGGSLRLSCAASGFTFSGYGMSWVR |
| | | antibody | QAPGKGLEWVSYISSGSYYTYYADSVKGRFTISRDNSKN |
| | | (T1-7) | TLYLQMNSLRAEDTAVYYCARYRGDNDYYGYFDYWGQG |
| | | | TLVTVSSGGGGSGGGGSGGGGSQSVLTQPPSASGTPG |
| | | | QRVTISCSCSSSNIGNNAVSWYQQLPGTAPKLLIYRNNQ |
| | | | RPSGVPDRFSGSKSGTSASLAISGLRSEDEADYYCAADY |
| | | | GSDYVFGGGTKLTVLG |
| 30 | Protein | scFv CAR of anti | EVQLLESGGGLVQPGGSLRLSCAASGFTFSGYGMSWVR |
| | | CD30 antibody | QAPGKGLEWVSYISSGSYYTYYADSVKGRFTISRDNSKN |
| | | (T1-7) | TLYLQMNSLRAEDTAVYYCARYRGDNDYYGYFDYWGQG |
| | | | TLVTVSSGGGGSGGGGSGGGGSQSVLTQPPSASGTPG |
| | | | QRVTISCSCSSSNIGNNAVSWYQQLPGTAPKLLIYRNNQ |
| | | | RPSGVPDRFSGSKSGTSASLAISGLRSEDEADYYCAADY |
| | | | GSDYVFGGGTKLTVLG |
| | | | GQAGQTTTPAPRPPTPAPTIASQPLSLRPEACRPAAGGA |
| | | | VHTRGLDFACDIYIWAPLAGTCGVLLLSLVITLYCKRGRKK |
| | | | LLYIFKQPFMRPVQTTQEEDGCSCRFPEEEEGGCELRVK |
| | | | FSRSADAPAYQQGQNQLYNELNLGRREEYDVLDKRRGR |
| | | | DPEMGGKPRRKNPQEGLYNELQKDKMAEAYSEIGMKGE |
| | | | RRRGKGHDGLYQGLSTATKDTYDALHMQALPPR |
| 31 | DNA | Nucleotide sequence | GGTTATGGTATGAGC |
| | | for encoding CDRH1 | |
| | | of anti CD30 antibody | |
| | | (T1-7) | |
| 32 | DNA | Nucleotide sequence | TACATCTCTTCTGGTTCTTACTACACGTATTACGCTGAT |
| | | for encoding CDRH2 | TCTGTAAAAGGT |
| | | of anti CD30 antibody | |
| | | (T1-7) | |
| 33 | DNA | Nucleotide sequence | TACCGTGGTGACAACGATTACTACGGTTACTTCGACTA |
| | | for encoding CDRH3 | C |
| | | of anti CD30 antibody | |
| | | (T1-7) | |
| 34 | DNA | Nucleotide sequence | AGTTGCTCTTCATCTAATATTGGCAATAATGCTGTCTCC |
| | | for encoding CDRL1 of | |
| | | anti CD30 antibody | |
| | | (T1-7) | |
| 35 | DNA | Nucleotide sequence | AGAAATAACCAGCGGCCAAGC |
| | | for encoding CDRL2 of | |
| | | anti CD30 antibody | |
| | | (T1-7) | |
| 36 | DNA | Nucleotide sequence | GCTGCTGACTACGGTTCTGAC |
| | | for encoding CDRL3 of | |
| | | anti CD30 antibody | |
| | | (T1-7) | |
| 37 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCGGTTATGGTATGAGCTGGGTCC |
| | | of anti CD30 antibody | GCCAGGCTCCAGGGAAGGGGCTGGAGTGGGTCTCAT |
| | | (T1-7) | ACATCTCTTCTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACCGT |
| | | | GGTGACAACGATTACTACGGTTACTTCGACTACTGGGG |
| | | | CCAGGGTACACTGGTCACCGTGAGCTCA |
| 38 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTTGCTC |
| | | chain variable domain | TTCATCTAATATTGGCAATAATGCTGTCTCCTGGTACCA |
| | | of anti CD30 antibody | GCAGCTCCCAGGAACGGCCCCCAAACTCCTCATCTAT |
| | | (T1-7) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACTACGGTTCTGACTATGTCTTCGG |
| | | | CGGAGGCACCAAGCTGACGGTCCTAGGT |
| 39 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCGGTTATGGTATGAGCTGGGTCC |
| | | (T1-7) | GCCAGGCTCCAGGGAAGGGGCTGGAGTGGGTCTCAT |
| | | | ACATCTCTTCTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACCGT |
| | | | GGTGACAACGATTACTACGGTTACTTCGACTACTGGGG |
| | | | CCAGGGTACACTGGTCACCGTGAGCTCAGGTGGAGGC |
| | | | GGTTCAGGCGGAGGTGGATCCGGCGGTGGCGGATCG |
| | | | CAGTCTGTGCTGACTCAGCCACCCTCAGCGTCTGGGA |
| | | | CCCCCGGGCAGAGGGTCACCATCTCTTGTAGTTGCTC |
| | | | TTCATCTAATATTGGCAATAATGCTGTCTCCTGGTACCA |
| | | | GCAGCTCCCAGGAACGGCCCCCAAACTCCTCATCTAT |
| | | | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACTACGGTTCTGACTATGTCTTCGG |
| | | | CGGAGGCACCAAGCTGACGGTCCTAGGT |
| 40 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCGGTTATGGTATGAGCTGGGTCC |
| | | antibody | GCCAGGCTCCAGGGAAGGGGCTGGAGTGGGTCTCAT |
| | | (T1-7) | ACATCTCTTCTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACCGT |
| | | | GGTGACAACGATTACTACGGTTACTTCGACTACTGGGG |
| | | | CCAGGGTACACTGGTCACCGTGAGCTCAGGTGGAGGC |
| | | | GGTTCAGGCGGAGGTGGATCCGGCGGTGGQGGATCG |
| | | | CAGTCTGTGCTGACTCAGCCACCCTCAGCGTCTGGGA |
| | | | CCCCCGGGCAGAGGGTCACCATCTCTTGTAGTTGCTC |
| | | | TTCATCTAATATTGGCAATAATGCTGTCTCCTGGTACCA |
| | | | GCAGCTCCCAGGAACGGCCCCCAAACTCCTCATCTAT |
| | | | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACTACGGTTCTGACTATGTCTTCGG |
| | | | CGGAGGCACCAAGCTGACGGTCCTAGGT |
| | | | GGCCAGGCCGGCCAGACAACGACACCTGCTCCCAGA |
| | | | CCGCCTACTCCCGCCCCAACCATTGCATCTCAGCCACT |
| | | | CTCTCTGAGACCCGAAGCGTGTAGACCTGCGGCCGGG |
| | | | GGCGCTGTCCACACAAGAGGCTTAGACTTCGCCTGCG |
| | | | ATATCTATATCTGGGCCCCACTCGCAGGCACTTGTGGA |
| | | | GTGCTGCTGCTTTCACTCGTGATAACCCTGTACTGCAA |
| | | | AAGGGGGAGAAAGAAGCTGCTGTATATTTTTAAACAAC |
| | | | CATTTATGAGACCTGTTCAGACTACCCAGGAAGAAGAC |
| | | | GGTTGTAGTTGCAGATTCCCCGAGGAGGAAGAAGGAG |
| | | | GTTGCGAGTTGAGAGTAAAGTTCAGCAGATCCGCAGAT |
| | | | GCCCCTGCTTACCAGCAGGGTCAAAACCAGCTTTACAA |
| | | | CGAGCTGAATTTAGGTAGAAGAGAGGAATATGACGTGT |
| | | | TGGATAAAAGAAGAGGAAGAGACCCGGAAATGGGCGG |
| | | | CAAGCCTCGAAGAAAAAATCCCCAAGAGGGACTCTACA |
| | | | ATGAGCTGCAGAAGGACAAAATGGCTGAAGCCTACAG |
| | | | CGAGATCGGCATGAAGGGAGAAAGACGCAGAGGGAAA |
| | | | GGGCATGATGGGCTTTATCAGGGCTTGTCCACCGCTA |
| | | | CAAAGGATACTTATGACGCACTACACATGCAGGCCCTG |
| | | | CCACCCCGT |
| 41 | Protein | CDRH1 of anti CD30 | SYMS |
| | | antibody (T1-8) | |
| 42 | Protein | CDRH2 of anti CD30 | SIGSGYYSTYYADSVKG |
| | | antibody (T1-8) | |
| 43 | Protein | CDRH3 of anti CD30 | DYYGGFDY |
| | | antibody (T1-8) | |
| 44 | Protein | CDRL1 of anti CD30 | SGSSSNIGSNYVY |
| | | antibody (T1-8) | |
| 45 | Protein | CDRL2 of anti CD30 | RNNQRPS |
| | | antibody (T1-8) | |
| 46 | Protein | CDRL3 of anti CD30 | AAYDSYS |
| | | antibody (T1-8) | |
| 47 | Protein | Heavy chain variable | EVQLLESGGGLVQPGGSLRLSCAASGFTFSSYMSWVRQ |
| | | domain of anti CD30 | APGKGLEWVSSIGSGYYSTYYADSVKGRFTISRDNSKNT |
| | | antibody (T1-8) | LYLQMNSLRAEDTAVYYCARDYYGGFDYWGQGTLVTVS |
| | | | S |
| 48 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAAYDSYSYVFGGGTKLTVLG |
| | | (T1-8) | |
| 49 | Protein | scFv of anti CD30 | EVQLLESGGGLVQPGGSLRLSCAASGFTFSSYMSWVRQ |
| | | antibody | APGKGLEWVSSIGSGYYSTYYADSVKGRFTISRDNSKNT |
| | | (T1-8) | LYLQMNSLRAEDTAVYYCARDYYGGFDYWGQGTLVTVS |
| | | | SGGGGSGGGGSGGGGSQSVLTQPPSASGTPGQRVTIS |
| | | | CSGSSSNIGSNYVYWYQQLPGTAPKLLIYRNNQRPSGVP |
| | | | DRFSGSKSGTSASLAISGLRSEDEADYYCAAYDSYSYVF |
| | | | GGGTKLTVLG |
| 50 | Protein | scFv CAR of anti | EVQLLESGGGLVQPGGSLRLSCAASGFTFSSYMSWVRQ |
| | | CD30 antibody | APGKGLEWVSSIGSGYYSTYYADSVKGRFTISRDNSKNT |
| | | (T1-8) | LYLQMNSLRAEDTAVYYCARDYYGGFDYWGQGTLVTVS |
| | | | SGGGGSGGGGSGGGGSQSVLTQPPSASGTPGQRVTIS |
| | | | CSGSSSNIGSNYVYWYQQLPGTAPKLLIYRNNQRPSGVP |
| | | | DRFSGSKSGTSASLAISGLRSEDEADYYCAAYDSYSYVF |
| | | | GGGTKLTVLGGQAGQTTTPAPRPPTPAPTIASQPLSLRP |
| | | | EACRPAAGGAVHTRGLDFACDIYIWAPLAGTCGVLLLSLV |
| | | | ITLYCKRGRKKLLYIFKQPFMRPVQTTQEEDGCSCRFPEE |
| | | | EEGGCELRVKFSRSADAPAYQQGQNQLYNELNLGRREE |
| | | | YDVLDKRRGRDPEMGGKPRRKNPQEGLYNELQKDKMA |
| | | | EAYSEIGMKGERRRGKGHDGLYQGLSTATKDTYDALHM |
| | | | QALPPR |
| 51 | DNA | Nucleotide sequence | TCTTACATGAGC |
| | | for encoding CDRH1 | |
| | | of anti CD30 antibody | |
| | | (T1-8) | |
| 52 | DNA | Nucleotide sequence | TCTATCGGTTCTGGTTACTACTCTACGTATTACGCTGAT |
| | | for encoding CDRH2 | TCTGTAAAAGGT |
| | | of anti CD30 antibody | |
| | | (T1-8) | |
| 53 | DNA | Nucleotide sequence | GACTACTACGGTGGTTTCGACTAC |
| | | for encoding CDRH3 | |
| | | of anti CD30 antibody | |
| | | (T1-8) | |
| 54 | DNA | Nucleotide sequence | AGTGGCTCTTCATCTAATATTGGCTCTAATTATGTCTAC |
| | | for encoding CDRL1 of | |
| | | anti CD30 antibody | |
| | | (T1-8) | |
| 55 | DNA | Nucleotide sequence | AGAAATAACCAGCGGCCAAGC |
| | | for encoding CDRL2 of | |
| | | anti CD30 antibody | |
| | | (T1-8) | |
| 56 | DNA | Nucleotide sequence | GCTGCTTACGACTCTTACTCT |
| | | for encoding CDRL3 of | |
| | | anti CD30 antibody | |
| | | (T1-8) | |
| 57 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTCTTACATGAGCTGGGTCCGCC |
| | | of anti CD30 antibody | AGGCTCCAGGGAAGGGGCTGGAGTGGGTCTCATCTAT |
| | | (T1-8) | CGGTTCTGGTTACTACTCTACGTATTACGCTGATTCTGT |
| | | | AAAAGGTCGGTTCACCATCTCCAGAGACAATTCCAAGA |
| | | | ACACGCTGTATCTGCAAATGAACAGCCTGAGAGCCGA |
| | | | GGACACGGCCGTGTATTACTGTGCGCGTGACTACTAC |
| | | | GGTGGTTTCGACTACTGGGGCCAGGGTACACTGGTCA |
| | | | CCGTGAGCTCA |
| 58 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 antibody | GCAGCTCCCAGGAACGGCCCCCAAACTCCTCATCTAT |
| | | (T1-8) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACGACTCTTACTCTTATGTCTTCGGC |
| | | | GGAGGCACCAAGCTGACGGTCCTAGGT |
| 59 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTCTTACATGAGCTGGGTCCGCC |
| | | (T1-8) | AGGCTCCAGGGAAGGGGCTGGAGTGGGTCTCATCTAT |
| | | | CGGTTCTGGTTACTACTCTACGTATTACGCTGATTCTGT |
| | | | AAAAGGTCGGTTCACCATCTCCAGAGACAATTCCAAGA |
| | | | ACACGCTGTATCTGCAAATGAACAGCCTGAGAGCCGA |
| | | | GGACACGGCCGTGTATTACTGTGCGCGTGACTACTAC |
| | | | GGTGGTTTCGACTACTGGGGCCAGGGTACACTGGTCA |
| | | | CCGTGAGCTCAGGTGGAGGCGGTTCAGGCGGAGGTG |
| | | | GATCCGGCGGTGGCGGATCGCAGTCTGTGCTGACTCA |
| | | | GCCACCCTCAGCGTCTGGGACCCCCGGGCAGAGGGT |
| | | | CACCATCTCTTGTAGTGGCTCTTCATCTAATATTGGCTC |
| | | | TAATTATGTCTACTGGTACCAGCAGCTCCCAGGAACGG |
| | | | CCCCCAAACTCCTCATCTATAGAAATAACCAGCGGCCA |
| | | | AGCGGGGTCCCTGACCGATTCTCTGGCTCCAAGTCTG |
| | | | GCACCTCAGCCTCCCTGGCCATCAGTGGGCTCCGGTC |
| | | | CGAGGATGAGGCCGATTATTACTGTGCTGCTTACGACT |
| | | | CTTACTCTTATGTCTTCGGCGGAGGCACCAAGCTGACG |
| | | | GTCCTAGGT |
| 60 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTCTTACATGAGCTGGGTCCGCC |
| | | antibody | AGGCTCCAGGGAAGGGGCTGGAGTGGGTCTCATCTAT |
| | | (T1-8) | CGGTTCTGGTTACTACTCTACGTATTACGCTGATTCTGT |
| | | | AAAAGGTCGGTTCACCATCTCCAGAGACAATTCCAAGA |
| | | | ACACGCTGTATCTGCAAATGAACAGCCTGAGAGCCGA |
| | | | GGACACGGCCGTGTATTACTGTGCGCGTGACTACTAC |
| | | | GGTGGTTTCGACTACTGGGGCCAGGGTACACTGGTCA |
| | | | CCGTGAGCTCAGGTGGAGGCGGTTCAGGCGGAGGTG |
| | | | GATCCGGCGGTGGCGGATCGCAGTCTGTGCTGACTCA |
| | | | GCCACCCTCAGCGTCTGGGACCCCCGGGCAGAGGGT |
| | | | CACCATCTCTTGTAGTGGCTCTTCATCTAATATTGGCTC |
| | | | TAATTATGTCTACTGGTACCAGCAGCTCCCAGGAACGG |
| | | | CCCCCAAACTCCTCATCTATAGAAATAACCAGCGGCCA |
| | | | AGCGGGGTCCCTGACCGATTCTCTGGCTCCAAGTCTG |
| | | | GCACCTCAGCCTCCCTGGCCATCAGTGGGCTCCGGTC |
| | | | CGAGGATGAGGCCGATTATTACTGTGCTGCTTACGACT |
| | | | CTTACTCTTATGTCTTCGGCGGAGGCACCAAGCTGACG |
| | | | GTCCTAGGTGGCCAGGCCGGCCAGACAACGACACCTG |
| | | | CTCCCAGACCGCCTACTCCCGCCCCAACCATTGCATCT |
| | | | CAGCCACTCTCTCTGAGACCCGAAGCGTGTAGACCTG |
| | | | CGGCCGGGGGCGCTGTCCACACAAGAGGCTTAGACTT |
| | | | CGCCTGCGATATCTATATCTGGGCCCCACTCGCAGGC |
| | | | ACTTGTGGAGTGCTGCTGCTTTCACTCGTGATAACCCT |
| | | | GTACTGCAAAAGGGGGAGAAAGAAGCTGCTGTATATTT |
| | | | TTAAACAACCATTTATGAGACCTGTTCAGACTACCCAG |
| | | | GAAGAAGACGGTTGTAGTTGCAGATTCCCCGAGGAGG |
| | | | AAGAAGGAGGTTGCGAGTTGAGAGTAAAGTTCAGCAG |
| | | | ATCCGCAGATGCCCCTGCTTACCAGCAGGGTCAAAAC |
| | | | CAGCTTTACAACGAGCTGAATTTAGGTAGAAGAGAGGA |
| | | | ATATGACGTGTTGGATAAAAGAAGAGGAAGAGACCCG |
| | | | GAAATGGGCGGCAAGCCTCGAAGAAAAAATCCCCAAG |
| | | | AGGGACTCTACAATGAGCTGCAGAAGGACAAAATGGC |
| | | | TGAAGCCTACAGCGAGATCGGCATGAAGGGAGAAAGA |
| | | | CGCAGAGGGAAAGGGCATGATGGGCTTTATCAGGGCT |
| | | | TGTCCACCGCTACAAAGGATACTTATGACGCACTACAC |
| | | | ATGCAGGCCCTGCCACCCCGT |
| 61 | Protein | CDRH1 of anti CD30 | YYGMS |
| | | antibody (T1-9) | |
| 62 | Protein | CDRH2 of anti CD30 | GIGSYSSYTYYADSVKG |
| | | antibody (T1-9) | |
| 63 | Protein | CDRH3 of anti CD30 | YASSPDAYFDY |
| | | antibody (T1-9) | |
| 64 | Protein | CDRL1 of anti CD30 | SGSSSNIGSNYVY |
| | | antibody (T1-9) | |
| 65 | Protein | CDRL2 of anti CD30 | RNNQRPS |
| | | antibody (T1-9) | |
| 66 | Protein | CDRL3 of anti CD30 | AAYYNYN |
| | | antibody (T1-9) | |
| 67 | Protein | Heavy chain variable | EVQLLESGGGLVQPGGSLRLSCAASGFTFSYYGMSWVR |
| | | domain of anti CD30 | QAPGKGLEWVSGIGSYSSYTYYADSVKGRFTISRDNSKN |
| | | antibody (T1-9) | TLYLQMNSLRAEDTAVYYCARYASSPDAYFDYWGQGTL |
| | | | VTVSS |
| 68 | Protein | Light chain variable | QSVLTQPPSASGTPGQRVTISCSGSSSNIGSNYVYWYQQ |
| | | domain of anti CD30 | LPGTAPKLLIYRNNQRPSGVPGRFSGSKSGTSASLAISGL |
| | | antibody | RSEDEADYYCAAYYNYNYVFGGGTKLTVLG |
| | | (T1-9) | |
| 69 | Protein | scFv of anti CD30 | EVQLLESGGGLVQPGGSLRLSCAASGFTFSYYGMSWVR |
| | | antibody | QAPGKGLEWVSGIGSYSSYTYYADSVKGRFTISRDNSKN |
| | | (T1-9) | TLYLQMNSLRAEDTAVYYCARYASSPDAYFDYWGQGTL |
| | | | VTVSSGGGGSGGGGSGGGGSQSVLTQPPSASGTPGQR |
| | | | VTISCSGSSSNIGSNYVYWYQQLPGTAPKLLIYRNNQRPS |
| | | | GVPGRFSGSKSGTSASLAISGLRSEDEADYYCAAYYNYN |
| | | | YVFGGGTKLTVLG |
| 70 | Protein | scFv CAR of anti | EVQLLESGGGLVQPGGSLRLSCAASGFTFSYYGMSWVR |
| | | CD30 antibody | QAPGKGLEWVSGIGSYSSYTYYADSVKGRFTISRDNSKN |
| | | (T1-9) | TLYLQMNSLRAEDTAVYYCARYASSPDAYFDYWGQGTL |
| | | | VTVSSGGGGSGGGGSGGGGSQSVLTQPPSASGTPGQR |
| | | | VTISCSGSSSNIGSNYVYWYQQLPGTAPKLLIYRNNQRPS |
| | | | GVPGRFSGSKSGTSASLAISGLRSEDEADYYCAAYYNYN |
| | | | YVFGGGTKLTVLGGQAGQTTTPAPRPPTPAPTIASQPLSL |
| | | | RPEACRPAAGGAVHTRGLDFACDIYIWAPLAGTCGVLLLS |
| | | | LVITLYCKRGRKKLLYIFKQPFMRPVQTTQEEDGCSCRFP |
| | | | EEEEGGCELRVKFSRSADAPAYQQGQNQLYNELNLGRR |
| | | | EEYDVLDKRRGRDPEMGGKPRRKNPQEGLYNELQKDK |
| | | | MAEAYSEIGMKGERRRGKGHDGLYQGLSTATKDTYDAL |
| | | | HMQALPPR |
| 71 | DNA | Nucleotide sequence | TACTATGGTATGAGC |
| | | for encoding CDRH1 | |
| | | of anti CD30 antibody | |
| | | (T1-9) | |
| 72 | DNA | Nucleotide sequence | GGTATCGGTTCTTACTCTTCTTACACGTATTACGCTGAT |
| | | for encoding CDRH2 | TCTGTAAAAGGT |
| | | of anti CD30 antibody | |
| | | (T1-9) | |
| 73 | DNA | Nucleotide sequence | TACGCTTCTTCTCCGGACGCTTACTTCGACTAC |
| | | for encoding CDRH3 | |
| | | of anti CD30 antibody | |
| | | (T1-9) | |
| 74 | DNA | Nucleotide sequence | AGTGGCTCTTCATCTAATATTGGCTCTAATTATGTCTAC |
| | | for encoding CDRL1 of | |
| | | anti CD30 antibody | |
| | | (T1-9) | |
| 75 | DNA | Nucleotide sequence | AGAAATAACCAGCGGCCAAGC |
| | | for encoding CDRL2 of | |
| | | anti CD30 antibody | |
| | | (T1-9) | |
| 76 | DNA | Nucleotide sequence | GCTGCTTACTACAACTACAAC |
| | | for encoding CDRL3 of | |
| | | anti CD30 antibody | |
| | | (T1-9) | |
| 77 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTACTATGGTATGAGCTGGGTCC |
| | | of anti CD30 | GCCAGGCTCCAGGGAAGGGGCTGGAGTGGGTCTCAG |
| | | antibody (T1-9) | GTATCGGTTCTTACTCTTCTTACACGTATTACGCTGATT |
| | | | CTGTAAAAGGTCGGTTCAC1ATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGCT |
| | | | TCTTCTCCGGACGCTTACTTCGACTACTGGGGCCAGG |
| | | | GTACACTGGTCACCGTGAGCTCA |
| 78 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T1-9) | AGAAATAACCAGCGGCCAAGCGGGGTCCCTGGCCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACTACAACTACAACTATGTCTTCGG |
| | | | CGGAGGCACCAAGCTGACGGTCCTAGGT |
| 79 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTACTATGGTATGAGCTGGGTCC |
| | | (T1-9) | GCCAGGCTCCAGGGAAGGGGCTGGAGTGGGTCTCAG |
| | | | GTATCGGTTCTTACTCTTCTTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGCT |
| | | | TCTTCTCCGGACGCTTACTTCGACTACTGGGGCCAGG |
| | | | GTACACTGGTCACCGTGAGCTCAGGTGGAGGCGGTTC |
| | | | AGGCGGAGGTGGATCCGGCGGTGGCGGATCGCAGTC |
| | | | TGTGCTGACTCAGCCACCCTCAGCGTCTGGGACCCCC |
| | | | GGGCAGAGGGTCACCATCTCTTGTAGTGGCTCTTCATC |
| | | | TAATATTGGCTCTAATTATGTCTACTGGTACCAGCAGCT |
| | | | CCCAGGAACGGCCCCCAAACTCCTCATCTATAGAAATA |
| | | | ACCAGCGGCCAAGCGGGGTCCCTGGCCGATTCTCTGG |
| | | | CTCCAAGTCTGGCACCTCAGCCTCCCTGGCCATCAGT |
| | | | GGGCTCCGGTCCGAGGATGAGGCCGATTATTACTGTG |
| | | | CTGCTTACTACAACTACAACTATGTCTTCGGCGGAGGC |
| | | | ACCAAGCTGACGGTCCTAGGT |
| 80 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTACTATGGTATGAGCTGGGTCC |
| | | antibody | GCCAGGCTCCAGGGAAGGGGCTGGAGTGGGTCTCAG |
| | | (T1-9) | GTATCGGTTCTTACTCTTCTTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGCT |
| | | | TCTTCTCCGGACGCTTACTTCGACTACTGGGGCCAGG |
| | | | GTACACTGGTCACCGTGAGCTCAGGTGGAGGCGGTTC |
| | | | AGGCGGAGGTGGATCCGGCGGTGGCGGATCGCAGTC |
| | | | TGTGCTGACTCAGCCACCCTCAGCGTCTGGGACCCCC |
| | | | GGGCAGAGGGTCACCATCTCTTGTAGTGGCTCTTCATC |
| | | | TAATATTGGCTCTAATTATGTCTACTGGTACCAGCAGCT |
| | | | CCCAGGAACGGCCCCCAAACTCCTCATCTATAGAAATA |
| | | | ACCAGCGGCCAAGCGGGGTCCCTGGCCGATTCTCTGG |
| | | | CTCCAAGTCTGGCACCTCAGCCTCCCTGGCCATCAGT |
| | | | GGGCTCCGGTCCGAGGATGAGGCCGATTATTACTGTG |
| | | | CTGCTTACTACAACTACAACTATGTCTTCGGCGGAGGC |
| | | | ACCAAGCTGACGGTCCTAGGTGGCCAGGCCGGCCAG |
| | | | ACAACGACACCTGCTCCCAGACCGCCTACTCCCGCCC |
| | | | CAACCATTGCATCTCAGCCACTCTCTCTGAGACCCGAA |
| | | | GCGTGTAGACCTGCGGCCGGGGGCGCTGTCCACACA |
| | | | AGAGGCTTAGACTTCGCCTGCGATATCTATATCTGGGC |
| | | | CCCACTCGCAGGCACTTGTGGAGTGCTGCTGCTTTCA |
| | | | CTCGTGATAACCCTGTACTGCAAAAGGGGGAGAAAGA |
| | | | AGCTGCTGTATATTTTTAAACAACCATTTATGAGACCTG |
| | | | TTCAGACTACCCAGGAAGAAGACGGTTGTAGTTGCAGA |
| | | | TTCCCCGAGGAGGAAGAAGGAGGTTGCGAGTTGAGAG |
| | | | TAAAGTTCAGCAGATCCGCAGATGCCCCTGCTTACCAG |
| | | | CAGGGTCAAAACCAGCTTTACAACGAGCTGAATTTAGG |
| | | | TAGAAGAGAGGAATATGACGTGTTGGATAAAAGAAGAG |
| | | | GAAGAGACCCGGAAATGGGGGGCAAGCCTCGAAGAAA |
| | | | AAATCCCCAAGAGGGACTCTACAATGAGCTGCAGAAG |
| | | | GACAAAATGGCTGAAGCCTACAGCGAGATCGGCATGA |
| | | | AGGGAGAAAGACGCAGAGGGAAAGGGCATGATGGGC |
| | | | TTTATCAGGGCTTGTCCACCGCTACAAAGGATACTTAT |
| | | | GACGCACTACACATGCAGGCCCTGCCACCCCGT |
| 81 | Protein | CDRH1 of anti CD30 | SYGMS |
| | | antibody (T1-10) | |
| 82 | Protein | CDRH2 of anti CD30 | YISGGSYYTYYADSVKG |
| | | antibody (T1-10) | |
| 83 | Protein | CDRH3 of anti CD30 | YGYGYYDGSFDY |
| | | antibody (T1-10) | |
| 84 | Protein | CDRL1 of anti CD30 | SGSSSNIGSNYVY |
| | | antibody (T1-10) | |
| 85 | Protein | CDRL2 of anti CD30 | RNNQRPS |
| | | antibody (T1-10) | |
| 86 | Protein | CDRL3 of anti CD30 | AADGPYN |
| | | antibody (T1-10) | |
| 87 | Protein | Heavy chain variable | EVQLLESGGGLVQPGGSLRLSCAASGFTFSSYGMSWVR |
| | | domain of anti CD30 | QAPGKGLEWVSYISGGSYYTYYADSVKGRFTISRDNSKN |
| | | antibody (T1-10) | TLYLQMNSLRAEDTAVYYCARYGYGYYDGSFDYWGQGT |
| | | | LVTVSS |
| 88 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody (T1-10) | RSEDEADYYCAADGPYNYVFGGGTKLTVLG |
| 89 | Protein | scFv of anti CD30 | EVQLLESGGGLVQPGGSLRLSCAASGFTFSSYGMSWVR |
| | | antibody | QAPGKGLEWVSYISGGSYYTYYADSVKGRFTISRDNSKN |
| | | (T1-10) | TLYLQMNSLRAEDTAVYYCARYGYGYYDGSFDYWGQGT |
| | | | LVTVSSGGGGSGGGGGGGGSQSVLTQPPSASGTPGQ |
| | | | RVTISCSGSSSNIGSNYVYWYQQLPGTAPKLLIYRNNQRP |
| | | | SGVPDRFSGSKSGTSASLAISGLRSEDEADYYCAADGPY |
| | | | NYVFGGGTKLTVLG |
| 90 | Protein | scFv CAR of anti | EVQLLESGGGLVQPGGSLRLSCAASGFTFSSYGMSWVR |
| | | CD30 antibody | QAPGKGLEWVSYISGGSYYTYYADSVKGRFTISRDNSKN |
| | | (T1-10) | TLYLQMNSLRAEDTAVYYCARYGYGYYDGSFDYWGQGT |
| | | | LVTVSSGGGGSGGGGSGGGGSQSVLTQPPSASGTPGQ |
| | | | RVTISCSGSSSNIGSNYVYWYQQLPGTAPKLLIYRNNQRP |
| | | | SGVPDRFSGSKSGTSASLAISGLRSEDEADYYCAADGPY |
| | | | NYVFGGGTKLTVLGGQAGQTTTPAPRPPTPAPTIASQPL |
| | | | SLRPEACRPAAGGAVHTRGLDFACDIYIWAPLAGTCGVLL |
| | | | LSLVITLYCKRGRKKLLYIFKQPFMRPVQTTQEEDGCSCR |
| | | | FPEEEEGGCELRVKFSRSADAPAYQQGQNQLYNELNLG |
| | | | RREEYDVLDKRRGRDPEMGGKPRRKNPQEGLYNELQK |
| | | | DKMAEAYSEIGMKGERRRGKGHDGLYQGLSTATKDTYD |
| | | | ALHMQALPPR |
| 91 | DNA | Nucleotide sequence | TCTTATGGTATGAGC |
| | | for encoding CDRH1 | |
| | | of anti CD30 antibody | |
| | | (T1-10) | |
| 92 | DNA | Nucleotide sequence | TACATCTCTGGTGGTTCTTACTACACGTATTACGCTGAT |
| | | for encoding CDRH2 | TCTGTAAAAGGT |
| | | of anti CD30 | |
| | | antibody (T1-10) | |
| 93 | DNA | Nucleotide sequence | TACGGTTACGGTTACTACGACGGTTCTTTCGACTAC |
| | | for encoding CDRH3 | |
| | | of anti CD30 | |
| | | antibody (T1-10) | |
| 94 | DNA | Nucleotide sequence | AGTGGCTCTTCATCTAATATTGGCTCTAATTATGTCTAC |
| | | for encoding CDRL1 | |
| | | of anti CD30 | |
| | | antibody (T1-10) | |
| 95 | DNA | Nucleotide sequence | AGAAATAACCAGCGGCCAAGC |
| | | for encoding CDRL2 | |
| | | of anti CD30 | |
| | | antibody (T1-10) | |
| 96 | DNA | Nucleotide sequence | GCTGCTGACGGTCCGTACAAC |
| | | for encoding CDRL3 | |
| | | of anti CD30 | |
| | | antibody (T1-10) | |
| 97 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTCTTATGGTATGAGCTGGGTCC |
| | | of anti CD30 | GCCAGGCTCCAGGGAAGGGGCTGGAGTGGGTCTCAT |
| | | antibody (T1-10) | ACATCTCTGGTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGGT |
| | | | TACGGTTACTACGACGGTTCTTTCGACTACTGGGGCCA |
| | | | GGGTACACTGGTCACCGTGAGCTCA |
| 98 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T1-10) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACGGTCCGTACAACTATGTCTTCGG |
| | | | CGGAGGCACCAAGCTGACGGTCCTAGGT |
| 99 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTCTTATGGTATGAGCTGGGTCC |
| | | (T1-10) | GCCAGGCTCCAGGGAAGGGGCTGGAGTGGGTCTCAT |
| | | | ACATCTCTGGTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGGT |
| | | | TACGGTTACTACGACGGTTCTTTCGACTACTGGGGCCA |
| | | | GGGTACACTGGTCACCGTGAGCTCAGGTGGAGGCGGT |
| | | | TCAGGCGGAGGTGGATCCGGCGGTGGCGGATCGCAG |
| | | | TCTGTGCTGACTCAGCCACCCTCAGCGTCTGGGACCC |
| | | | CCGGGCAGAGGGTCACCATCTCTTGTAGTGGCTCTTC |
| | | | ATCTAATATTGGCTCTAATTATGTCTACTGGTACCAGCA |
| | | | GCTCCCAGGAACGGCCCCCAAACTCCTCATCTATAGAA |
| | | | ATAACCAGCGGCCAAGCGGGGTCCCTGACCGATTCTC |
| | | | TGGCTCCAAGTCTGGCACCTCAGCCTCCCTGGCCATC |
| | | | AGTGGGCTCCGGTCCGAGGATGAGGCCGATTATTACT |
| | | | GTGCTGCTGACGGTCCGTACAACTATGTCTTCGGCGG |
| | | | AGGCACCAAGCTGACGGTCCTAGGT |
| 100 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTCTTATGGTATGAGCTGGGTCC |
| | | antibody | GCCAGGCTCCAGGGAAGGGGCTGGAGTGGGTCTCAT |
| | | (T1-10) | ACATCTCTGGTGGTTCTTACTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACGGT |
| | | | TACGGTTACTACGACGGTTCTTTCGACTACTGGGGCCA |
| | | | GGGTACACTGGTCACCGTGAGCTCAGGTGGAGGCGGT |
| | | | TCAGGCGGAGGTGGATCCGGCGGTGGQGGATCGCAG |
| | | | TCTGTGCTGACTCAGCCACCCTCAGCGTCTGGGACCC |
| | | | CCGGGCAGAGGGTCACCATCTCTTGTAGTGGCTCTTC |
| | | | ATCTAATATTGGCTCTAATTATGTCTACTGGTACCAGCA |
| | | | GCTCCCAGGAACGGCCCCCAAACTCCTCATCTATAGAA |
| | | | ATAACCAGCGGCCAAGCGGGGTCCCTGACCGATTCTC |
| | | | TGGCTCCAAGTCTGGCACCTCAGCCTCCCTGGCCATC |
| | | | AGTGGGCTCCGGTCCGAGGATGAGGCCGATTATTACT |
| | | | GTGCTGCTGACGGTCCGTACAACTATGTCTTCGGCGG |
| | | | AGGCACCAAGCTGACGGTCCTAGGTGGCCAGGCCGG |
| | | | CCAGACAACGACACCTGCTCCCAGACCGCCTACTCCC |
| | | | GCCCCAACCATTGCATCTCAGCCACTCTCTCTGAGACC |
| | | | CGAAGCGTGTAGACCTGCGGCCGGGGGCGCTGTCCA |
| | | | CACAAGAGGCTTAGACTTCGCCTGCGATATCTATATCT |
| | | | GGGCCCCACTCGCAGGCACTTGTGGAGTGCTGCTGCT |
| | | | TTCACTCGTGATAACCCTGTACTGCAAAAGGGGGAGAA |
| | | | AGAAGCTGCTGTATATTTTTAAACAACCATTTATGAGAC |
| | | | CTGTTCAGACTACCCAGGAAGAAGACGGTTGTAGTTGC |
| | | | AGATTCCCCGAGGAGGAAGAAGGAGGTTGCGAGTTGA |
| | | | GAGTAAAGTTCAGCAGATCCGCAGATGCCCCTGCTTAC |
| | | | CAGCAGGGTCAAAACCAGCTTTACAACGAGCTGAATTT |
| | | | AGGTAGAAGAGAGGAATATGACGTGTTGGATAAAAGAA |
| | | | GAGGAAGAGACCCGGAAATGGGGGGCAAGCCTCGAA |
| | | | GAAAAAATCCCCAAGAGGGACTCTACAATGAGCTGCA |
| | | | GAAGGACAAAATGGCTGAAGCCTACAGCGAGATCGGC |
| | | | ATGAAGGGAGAAAGACGCAGAGGGAAAGGGCATGATG |
| | | | GGCTTTATCAGGGCTTGTCCACCGCTACAAAGGATACT |
| | | | TATGACGCACTACACATGCAGGCCCTGCCACCCCGT |
| 101 | Protein | CDRH1 of anti CD30 | SYSMS |
| | | antibody (T1-23) | |
| 102 | Protein | CDRH2 of anti CD30 | GIGYPYYTYYADSVKG |
| | | antibody (T1-23) | |
| 103 | Protein | CDRH3 of anti CD30 | YYYDYGFDY |
| | | antibody (T1-23) | |
| 104 | Protein | CDRL1 of anti CD30 | SGSSSNIGSNYVY |
| | | antibody (T1-23) | |
| 105 | Protein | CDRL2 of anti CD30 | RNNQRPS |
| | | antibody (T1-23) | |
| 106 | Protein | CDRL3 of anti CD30 | AAYRSYD |
| | | antibody (T1-23) | |
| 107 | Protein | Heavy chain variable | EVQLLESGGGLVQPGGSLRLSCAASGFTFSSYSMSWVR |
| | | domain of anti CD30 | QAPGKGLEWVSGIGYPYYTYYADSVKGRFTISRDNSKNT |
| | | antibody (T1-23) | LYLQMNSLRAEDTAVYYCARYYYDYGFDYWGQGTLVTV |
| | | | SS |
| 108 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAAYRSYDYVFGGGTKLTVLG |
| | | (T1-23) | |
| 109 | Protein | scFv of anti CD30 | EVQLLESGGGLVQPGGSLRLSCAASGFTFSSYSMSWVR |
| | | antibody | QAPGKGLEWVSGIGYPYYTYYADSVKGRFTISRDNSKNT |
| | | (T1-23) | LYLQMNSLRAEDTAVYYCARYYYDYGFDYWGQGTLVTV |
| | | | SSGGGGSGGGGSGGGGSQSVLTQPPSASGTPGQRVTI |
| | | | SCSGSSSNIGSNYVYWYQQLPGTAPKLLIYRNNQRPSGV |
| | | | PDRFSGSKSGTSASLAISGLRSEDEADYYCAAYRSYDYV |
| | | | FGGGTKLTVLG |
| 110 | Protein | scFv CAR of anti | EVQLLESGGGLVQPGGSLRLSCAASGFTFSSYSMSWVR |
| | | CD30 antibody | QAPGKGLEWVSGIGYPYYTYYADSVKGRFTISRDNSKNT |
| | | (T1-23) | LYLQMNSLRAEDTAVYYCARYYYDYGFDYWGQGTLVTV |
| | | | SSGGGGSGGGGSGGGGSQSVLTQPPSASGTPGQRVTI |
| | | | SCSGSSSNIGSNYVYWYQQLPGTAPKLLIYRNNQRPSGV |
| | | | PDRFSGSKSGTSASLAISGLRSEDEADYYCAAYRSYDYV |
| | | | FGGGTKLTVLGGQAGQTTTPAPRPPTPAPTIASQPLSLRP |
| | | | EACRPAAGGAVHTRGLDFACDIYIWAPLAGTCGVLLLSLV |
| | | | ITLYCKRGRKKLLYIFKQPFMRPVQTTQEEDGCSCRFPEE |
| | | | EEGGCELRVKFSRSADAPAYQQGQNQLYNELNLGRREE |
| | | | YDVLDKRRGRDPEMGGKPRRKNPQEGLYNELQKDKMA |
| | | | EAYSEIGMKGERRRGKGHDGLYQGLSTATKDTYDALHM |
| | | | QALPPR |
| 111 | DNA | Nucleotide sequence | TCTTATTCTATGAGC |
| | | for encoding CDRH1 | |
| | | of anti CD30 | |
| | | antibody (T1-23) | |
| 112 | DNA | Nucleotide sequence | GGTATCGGTTACCCTTACTACACGTATTACGCTGATTC |
| | | for encoding CDRH2 | TGTAAAAGGT |
| | | of anti CD30 | |
| | | antibody (T1-23) | |
| 113 | DNA | Nucleotide sequence | TACTACTACGACTACGGTTTCGACTAC |
| | | for encoding CDRH3 | |
| | | of anti CD30 | |
| | | antibody (T1-23) | |
| 114 | DNA | Nucleotide sequence | AGTGGCTCTTCATCTAATATTGGCTCTAATTATGTCTAC |
| | | for encoding CDRL1 | |
| | | of anti CD30 | |
| | | antibody (T1-23) | |
| 115 | DNA | Nucleotide sequence | AGAAATAACCAGCGGCCAAGC |
| | | for encoding CDRL2 | |
| | | of anti CD30 | |
| | | antibody (T1-23) | |
| 116 | DNA | Nucleotide sequence | GCTGCTTACCGTTCTTACGAC |
| | | for encoding CDRL3 | |
| | | of anti CD30 | |
| | | antibody (T1-23) | |
| 117 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTCTTATTCTATGAGCTGGGTCC |
| | | of anti CD30 | GCCAGGCTCCAGGGAAGGGGCTGGAGTGGGTCTCAG |
| | | antibody (T1-23) | GTATCGGTTACCCTTACTACACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTACTA |
| | | | CGACTACGGTTTCGACTACTGGGGCCAGGGTACACTG |
| | | | GTCACCGTGAGCTCA |
| 118 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T1-23) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACCGTTCTTACGACTATGTCTTCGG |
| | | | CGGAGGCACCAAGCTGACGGTCCTAGGT |
| 119 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTCTTATTCTATGAGCTGGGTCC |
| | | (T1-23) | GCCAGGCTCCAGGGAAGGGGCTGGAGTGGGTCTCAG |
| | | | GTATCGGTTACCCTTACTACACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTACTA |
| | | | CGACTACGGTTTCGACTACTGGGGCCAGGGTACACTG |
| | | | GTCACCGTGAGCTCAGGTGGAGGCGGTTCAGGCGGA |
| | | | GGTGGATCCGGCGGTGGCGGATCGCAGTCTGTGCTG |
| | | | ACTCAGCCACCCTCAGCGTCTGGGACCCCCGGGCAGA |
| | | | GGGTCACCATCTCTTGTAGTGGCTCTTCATCTAATATT |
| | | | GGCTCTAATTATGTCTACTGGTACCAGCAGCTCCCAGG |
| | | | AACGGCCCCCAAACTCCTCATCTATAGAAATAACCAGC |
| | | | GGCCAAGCGGGGTCCCTGACCGATTCTCTGGCTCCAA |
| | | | GTCTGGCACCTCAGCCTCCCTGGCCATCAGTGGGCTC |
| | | | CGGTCCGAGGATGAGGCCGATTATTACTGTGCTGCTTA |
| | | | CCGTTCTTACGACTATGTCTTCGGCGGAGGCACCAAG |
| | | | CTGACGGTCCTAGGT |
| 120 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTCTTATTCTATGAGCTGGGTCC |
| | | antibody | GCCAGGCTCCAGGGAAGGGGCTGGAGTGGGTCTCAG |
| | | (T1-23) | GTATCGGTTACCCTTACTACACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTACTA |
| | | | CGACTACGGTTTCGACTACTGGGGCCAGGGTACACTG |
| | | | GTCACCGTGAGCTCAGGTGGAGGCGGTTCAGGCGGA |
| | | | GGTGGATCCGGCGGTGGCGGATCGCAGTCTGTGCTG |
| | | | ACTCAGCCACCCTCAGCGTCTGGGACCCCCGGGCAGA |
| | | | GGGTCACCATCTCTTGTAGTGGCTCTTCATCTAATATT |
| | | | GGCTCTAATTATGTCTACTGGTACCAGCAGCTCCCAGG |
| | | | AACGGCCCCCAAACTCCTCATCTATAGAAATAACCAGC |
| | | | GGCCAAGCGGGGTCCCTGACCGATTCTCTGGCTCCAA |
| | | | GTCTGGCACCTCAGCCTCCCTGGCCATCAGTGGGCTC |
| | | | CGGTCCGAGGATGAGGCCGATTATTACTGTGCTGCTTA |
| | | | CCGTTCTTACGACTATGTCTTCGGCGGAGGCACCAAG |
| | | | CTGACGGTCCTAGGTGGCCAGGCCGGCCAGACAACG |
| | | | ACACCTGCTCCCAGACCGCCTACTCCCGCCCCAACCA |
| | | | TTGCATCTCAGCCACTCTCTCTGAGACCCGAAGCGTGT |
| | | | AGACCTGCGGCCGGGGGCGCTGTCCACACAAGAGGC |
| | | | TTAGACTTCGCCTGCGATATCTATATCTGGGCCCCACT |
| | | | CGCAGGCACTTGTGGAGTGCTGCTGCTTTCACTCGTG |
| | | | ATAACCCTGTACTGCAAAAGGGGGAGAAAGAAGCTGC |
| | | | TGTATATTTTTAAACAACCATTTATGAGACCTGTTCAGA |
| | | | CTACCCAGGAAGAAGACGGTTGTAGTTGCAGATTCCC |
| | | | CGAGGAGGAAGAAGGAGGTTGCGAGTTGAGAGTAAAG |
| | | | TTCAGCAGATCCGCAGATGCCCCTGCTTACCAGCAGG |
| | | | GTCAAAACCAGCTTTACAACGAGCTGAATTTAGGTAGA |
| | | | AGAGAGGAATATGACGTGTTGGATAAAAGAAGAGGAA |
| | | | GAGACCCGGAAATGGGCGGCAAGCCTCGAAGAAAAAA |
| | | | TCCCCAAGAGGGACTCTACAATGAGCTGCAGAAGGAC |
| | | | AAAATGGCTGAAGCCTACAGCGAGATCGGCATGAAGG |
| | | | GAGAAAGACGCAGAGGGAAAGGGCATGATGGGCTTTA |
| | | | TCAGGGCTTGTCCACCGCTACAAAGGATACTTATGACG |
| | | | CACTACACATGCAGGCCCTGCCACCCCGT |
| 121 | Protein | CDRH1 of anti CD30 | YYYMS |
| | | antibody (T1-36) | |
| 122 | Protein | CDRH2 of anti CD30 | YIGGGGSGTYYADSVKG |
| | | antibody (T1-36) | |
| 123 | Protein | CDRH3 of anti CD30 | GPYYGYFDY |
| | | antibody (T1-36) | |
| 124 | Protein | CDRL1 of anti CD30 | SGSSSNIGSNYVY |
| | | antibody (T1-36) | |
| 125 | Protein | CDRL2 of anti CD30 | RNNQRPS |
| | | antibody (T1-36) | |
| 126 | Protein | CDRL3 of anti CD30 | AAYPSYDS |
| | | antibody (T1-36) | |
| 127 | Protein | Heavy chain variable | EVQLLESGGGLVQPGGSLRLSCAASGFTFSYYYMSWVR |
| | | domain of anti CD30 | QAPGKGLEWVSYIGGGGSGTYYADSVKGRFTISRDNSK |
| | | antibody (T1-36) | NTLYLQMNSLRAEDTAVYYCARGPYYGYFDYWGQGTLV |
| | | | TVSS |
| 128 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAAYPSYDSYVFGGGTKLTVLG |
| | | (T1-36) | |
| 129 | Protein | scFv of anti CD30 | EVQLLESGGGLVQPGGSLRLSCAASGFTFSYYYMSWVR |
| | | antibody | QAPGKGLEWVSYIGGGGSGTYYADSVKGRFTISRDNSK |
| | | (T1-36) | NTLYLQMNSLRAEDTAVYYCARGPYYGYFDYWGQGTLV |
| | | | TVSSGGGGSGGGGSGGGGSQSVLTQPPSASGTPGQRV |
| | | | TISCSGSSSNIGSNYVYWYQQLPGTAPKLLIYRNNQRPSG |
| | | | VPDRFSGSKSGTSASLAISGLRSEDEADYYCAAYPSYDS |
| | | | YVFGGGTKLTVLG |
| 130 | Protein | scFv CAR of anti | EVQLLESGGGLVQPGGSLRLSCAASGFTFSYYYMSWVR |
| | | CD30 antibody | QAPGKGLEWVSYIGGGGSGTYYADSVKGRFTISRDNSK |
| | | (T1-36) | NTLYLQMNSLRAEDTAVYYCARGPYYGYFDYWGQGTLV |
| | | | TVSSGGGGSGGGGSGGGGSQSVLTQPPSASGTPGQRV |
| | | | TISCSGSSSNIGSNYVYWYQQLPGTAPKLLIYRNNQRPSG |
| | | | VPDRFSGSKSGTSASLAISGLRSEDEADYYCAAYPSYDS |
| | | | YVFGGGTKLTVLGGQAGQTTTPAPRPPTPAPTIASQPLSL |
| | | | RPEACRPAAGGAVHTRGLDFACDIYIWAPLAGTCGVLLLS |
| | | | LVITLYCKRGRKKLLYIFKQPFMRPVQTTQEEDGCSCRFP |
| | | | EEEEGGCELRVKFSRSADAPAYQQGQNQLYNELNLGRR |
| | | | EEYDVLDKRRGRDPEMGGKPRRKNPQEGLYNELQKDK |
| | | | MAEAYSEIGMKGERRRGKGHDGLYQGLSTATKDTYDAL |
| | | | HMQALPPR |
| 131 | DNA | Nucleotide sequence | TACTATTACATGAGC |
| | | for encoding CDRH1 | |
| | | of anti CD30 | |
| | | antibody (T1-36) | |
| 132 | DNA | Nucleotide sequence | TACATCGGTGGTGGTGGTTCTGGTACGTATTACGCTGA |
| | | for encoding CDRH2 | TTCTGTAAAAGGT |
| | | of anti CD30 | |
| | | antibody (T1-36) | |
| 133 | DNA | Nucleotide sequence | GGTCCGTACTACGGTTACTTCGACTAC |
| | | for encoding CDRH3 | |
| | | of anti CD30 | |
| | | antibody (T1-36) | |
| 134 | DNA | Nucleotide sequence | AGTGGCTCTTCATCTAATATTGGCTCTAATTATGTCTAC |
| | | for encoding CDRL1 | |
| | | of anti CD30 | |
| | | antibody(T1-36) | |
| 135 | DNA | Nucleotide sequence | AGAAATAACCAGCGGCCAAGC |
| | | for encoding CDRL2 | |
| | | of anti CD30 | |
| | | antibody (T1-36) | |
| 136 | DNA | Nucleotide sequence | GCTGCTTACCCGTCTTACGACTCT |
| | | for encoding CDRL3 | |
| | | of anti CD30 | |
| | | antibody (T1-36) | |
| 137 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTACTATTACATGAGCTGGGTCC |
| | | of anti CD30 | GCCAGGCTCCAGGGAAGGGGCTGGAGTGGGTCTCAT |
| | | antibody (T1-36) | ACATCGGTGGTGGTGGTTCTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTGGTCC |
| | | | GTACTACGGTTACTTCGACTACTGGGGCCAGGGTACA |
| | | | CTGGTCACCGTGAGCTCA |
| 138 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T1-36) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACCCGTCTTACGACTCTTATGTCTTC |
| | | | GGCGGAGGCACCAAGCTGACGGTCCTAGGT |
| 139 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTACTATTACATGAGCTGGGTCC |
| | | (T1-36) | GCCAGGCTCCAGGGAAGGGGCTGGAGTGGGTCTCAT |
| | | | ACATCGGTGGTGGTGGTTCTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTGGTCC |
| | | | GTACTACGGTTACTTCGACTACTGGGGCCAGGGTACA |
| | | | CTGGTCACCGTGAGCTCAGGTGGAGGCGGTTCAGGC |
| | | | GGAGGTGGATCCGGCGGTGGCGGATCGCAGTCTGTG |
| | | | CTGACTCAGCCACCCTCAGCGTCTGGGACCCCCGGGC |
| | | | AGAGGGTCACCATCTCTTGTAGTGGCTCTTCATCTAAT |
| | | | ATTGGCTCTAATTATGTCTACTGGTACCAGCAGCTCCC |
| | | | AGGAACGGCCCCCAAACTCCTCATCTATAGAAATAACC |
| | | | AGCGGCCAAGCGGGGTCCCTGACCGATTCTCTGGCTC |
| | | | CAAGTCTGGCACCTCAGCCTCCCTGGCCATCAGTGGG |
| | | | CTCCGGTCCGAGGATGAGGCCGATTATTACTGTGCTG |
| | | | CTTACCCGTCTTACGACTCTTATGTCTTCGGCGGAGGC |
| | | | ACCAAGCTGACGGTCCTAGGT |
| 140 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTACTATTACATGAGCTGGGTCC |
| | | antibody | GCCAGGCTCCAGGGAAGGGGCTGGAGTGGGTCTCAT |
| | | (T1-36) | ACATCGGTGGTGGTGGTTCTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTGGTCC |
| | | | GTACTACGGTTACTTCGACTACTGGGGCCAGGGTACA |
| | | | CTGGTCACCGTGAGCTCAGGTGGAGGCGGTTCAGGC |
| | | | GGAGGTGGATCCGGCGGTGGCGGATCGCAGTCTGTG |
| | | | CTGACTCAGCCACCCTCAGCGTCTGGGACCCCCGGGC |
| | | | AGAGGGTCACCATCTCTTGTAGTGGCTCTTCATCTAAT |
| | | | ATTGGCTCTAATTATGTCTACTGGTACCAGCAGCTCCC |
| | | | AGGAACGGCCCCCAAACTCCTCATCTATAGAAATAACC |
| | | | AGCGGCCAAGCGGGGTCCCTGACCGATTCTCTGGCTC |
| | | | CAAGTCTGGCACCTCAGCCTCCCTGGCCATCAGTGGG |
| | | | CTCCGGTCCGAGGATGAGGCCGATTATTACTGTGCTG |
| | | | CTTACCCGTCTTACGACTCTTATGTCTTCGGCGGAGGC |
| | | | ACCAAGCTGACGGTCCTAGGTGGCCAGGCCGGCCAG |
| | | | ACAACGACACCTGCTCCCAGACCGCCTACTCCCGCCC |
| | | | CAACCATTGCATCTCAGCCACTCTCTCTGAGACCCGAA |
| | | | GCGTGTAGACCTGCGGCCGGGGGCGCTGTCCACACA |
| | | | AGAGGCTTAGACTTCGCCTGCGATATCTATATCTGGGC |
| | | | CCCACTCGCAGGCACTTGTGGAGTGCTGCTGCTTTCA |
| | | | CTCGTGATAACCCTGTACTGCAAAAGGGGGAGAAAGA |
| | | | AGCTGCTGTATATTTTTAAACAACCATTTATGAGACCTG |
| | | | TTCAGACTACCCAGGAAGAAGACGGTTGTAGTTGCAGA |
| | | | TTCCCCGAGGAGGAAGAAGGAGGTTGCGAGTTGAGAG |
| | | | TAAAGTTCAGCAGATCCGCAGATGCCCCTGCTTACCAG |
| | | | CAGGGTCAAAACCAGCTTTACAACGAGCTGAATTTAGG |
| | | | TAGAAGAGAGGAATATGACGTGTTGGATAAAAGAAGAG |
| | | | GAAGAGACCCGGAAATGGGCGGCAAGCCTCGAAGAAA |
| | | | AAATCCCCAAGAGGGACTCTACAATGAGCTGCAGAAG |
| | | | GACAAAATGGCTGAAGCCTACAGCGAGATCGGCATGA |
| | | | AGGGAGAAAGACGCAGAGGGAAAGGGCATGATGGGC |
| | | | TTTATCAGGGCTTGTCCACCGCTACAAAGGATACTTAT |
| | | | GACGCACTACACATGCAGGCCCTGCCACCCCGT |
| 141 | Protein | CDRH1 of anti CD30 | YYGMS |
| | | antibody (T1-117) | |
| 142 | Protein | CDRH2 of anti CD30 | YISGYSSYTYYADSVKG |
| | | antibody (T1-117) | |
| 143 | Protein | CDRH3 of anti CD30 | YNDSGSFDY |
| | | antibody (T1-117) | |
| 144 | Protein | CDRL1 of anti CD30 | SGSSSNIGSNYVY |
| | | antibody (T1-117) | |
| 145 | Protein | CDRL2 of anti CD30 | RNNQRPS |
| | | antibody (T1-117) | |
| 146 | Protein | CDRL3 of anti CD30 | AADAGNR |
| | | antibody (T1-117) | |
| 147 | Protein | Heavy chain variable | EVQLLESGGGLVQPGGSLRLSCAASGFTFSYYGMSWVR |
| | | domain of anti CD30 | QAPGKGLEWVSYISGYSSYTYYADSVKGRFTISRDNSKN |
| | | antibody | TLYLQMNSLRAEDTAVYYCARYNDSGSFDYWGQGTLVT |
| | | (T1-117) | VSS |
| 148 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAADAGNRYVFGGGTKLTVLG |
| | | (T1-117) | |
| 149 | Protein | scFv of anti CD30 | EVQLLESGGGLVQPGGSLRLSCAASGFTFSYYGMSWVR |
| | | antibody | QAPGKGLEWVSYISGYSSYTYYADSVKGRFTISRDNSKN |
| | | (T1-117) | TLYLQMNSLRAEDTAVYYCARYNDSGSFDYWGQGTLVT |
| | | | VSSGGGGSGGGGSGGGGSQSVLTQPPSASGTPGQRVT |
| | | | ISCSGSSSNIGSNYVYWYQQLPGTAPKLLIYRNNQRPSG |
| | | | VPDRFSGSKSGTSASLAISGLRSEDEADYYCAADAGNRY |
| | | | VFGGGTKLTVLG |
| 150 | Protein | scFv CAR of anti | EVQLLESGGGLVQPGGSLRLSCAASGFTFSYYGMSWVR |
| | | CD30 antibody | QAPGKGLEWVSYISGYSSYTYYADSVKGRFTISRDNSKN |
| | | (T1-117) | TLYLQMNSLRAEDTAVYYCARYNDSGSFDYWGQGTLVT |
| | | | VSSGGGGSGGGGSGGGGSQSVLTQPPSASGTPGQRVT |
| | | | ISCSGSSSNIGSNYVYWYQQLPGTAPKLLIYRNNQRPSG |
| | | | VPDRFSGSKSGTSASLAISGLRSEDEADYYCAADAGNRY |
| | | | VFGGGTKLTVLGGQAGQTTTPAPRPPTPAPTIASQPLSLR |
| | | | PEACRPAAGGAVHTRGLDFACDIYIWAPLAGTCGVLLLSL |
| | | | VITLYCKRGRKKLLYIFKQPFMRPVQTTQEEDGCSCRFPE |
| | | | EEEGGCELRVKFSRSADAPAYQQGQNQLYNELNLGRRE |
| | | | EYDVLDKRRGRDPEMGGKPRRKNPQEGLYNELQKDKM |
| | | | AEAYSEIGMKGERRRGKGHDGLYQGLSTATKDTYDALH |
| | | | MQALPPR |
| 151 | DNA | Nucleotide sequence | TACTATGGTATGAGC |
| | | for encoding CDRH1 | |
| | | of anti CD30 | |
| | | antibody (T1-117) | |
| 152 | DNA | Nucleotide sequence | TACATCTCTGGTTACTCTTCTTACACGTATTACGCTGAT |
| | | for encoding CDRH2 | TCTGTAAAAGGT |
| | | of anti CD30 | |
| | | antibody (T1-117) | |
| 153 | DNA | Nucleotide sequence | TACAACGACTCTGGTTCTTTCGACTAC |
| | | for encoding CDRH3 | |
| | | of anti CD30 | |
| | | antibody (T1-117) | |
| 154 | DNA | Nucleotide sequence | AGTGGCTCTTCATCTAATATTGGCTCTAATTATGTCTAC |
| | | for encoding CDRL1 | |
| | | of anti CD30 | |
| | | antibody (T1-117) | |
| 155 | DNA | Nucleotide sequence | AGAAATAACCAGCGGCCAAGC |
| | | for encoding CDRL2 | |
| | | of anti CD30 | |
| | | antibody (T1-117) | |
| 156 | DNA | Nucleotide sequence | GCTGCTGACGCTGGTAACCGT |
| | | for encoding CDRL3 | |
| | | of anti CD30 | |
| | | antibody (T1-117) | |
| 157 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTACTATGGTATGAGCTGGGTCC |
| | | of anti CD30 | GCCAGGCTCCAGGGAAGGGGCTGGAGTGGGTCTCAT |
| | | antibody (T1-117) | ACATCTCTGGTTACTCTTCTTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACAAC |
| | | | GACTCTGGTTCTTTCGACTACTGGGGCCAGGGTACACT |
| | | | GGTCACCGTGAGCTCA |
| 158 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody(T1-117) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTGACGCTGGTAACCGTTATGTCTTCGG |
| | | | CGGAGGCACCAAGCTGACGGTCCTAGGT |
| 159 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTACTATGGTATGAGCTGGGTCC |
| | | (T1-117) | GCCAGGCTCCAGGGAAGGGGCTGGAGTGGGTCTCAT |
| | | | ACATCTCTGGTTACTCTTCTTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACAAC |
| | | | GACTCTGGTTCTTTCGACTACTGGGGCCAGGGTACACT |
| | | | GGTCACCGTGAGCTCAGGTGGAGGCGGTTCAGGCGG |
| | | | AGGTGGATCCGGCGGTGGCGGATCGCAGTCTGTGCT |
| | | | GACTCAGCCACCCTCAGCGTCTGGGACCCCCGGGCA |
| | | | GAGGGTCACCATCTCTTGTAGTGGCTCTTCATCTAATA |
| | | | TTGGCTCTAATTATGTCTACTGGTACCAGCAGCTCCCA |
| | | | GGAACGGCCCCCAAACTCCTCATCTATAGAAATAACCA |
| | | | GCGGCCAAGCGGGGTCCCTGACCGATTCTCTGGCTCC |
| | | | AAGTCTGGCACCTCAGCCTCCCTGGCCATCAGTGGGC |
| | | | TCCGGTCCGAGGATGAGGCCGATTATTACTGTGCTGC |
| | | | TGACGCTGGTAACCGTTATGTCTTCGGCGGAGGCACC |
| | | | AAGCTGACGGTCCTAGGT |
| 160 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTACTATGGTATGAGCTGGGTCC |
| | | antibody | GCCAGGCTCCAGGGAAGGGGCTGGAGTGGGTCTCAT |
| | | (T1-117) | ACATCTCTGGTTACTCTTCTTACACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTTACAAC |
| | | | GACTCTGGTTCTTTCGACTACTGGGGCCAGGGTACACT |
| | | | GGTCACCGTGAGCTCAGGTGGAGGCGGTTCAGGCGG |
| | | | AGGTGGATCCGGCGGTGGCGGATCGCAGTCTGTGCT |
| | | | GACTCAGCCACCCTCAGCGTCTGGGACCCCCGGGCA |
| | | | GAGGGTCACCATCTCTTGTAGTGGCTCTTCATCTAATA |
| | | | TTGGCTCTAATTATGTCTACTGGTACCAGCAGCTCCCA |
| | | | GGAACGGCCCCCAAACTCCTCATCTATAGAAATAACCA |
| | | | GCGGCCAAGCGGGGTCCCTGACCGATTCTCTGGCTCC |
| | | | AAGTCTGGCACCTCAGCCTCCCTGGCCATCAGTGGGC |
| | | | TCCGGTCCGAGGATGAGGCCGATTATTACTGTGCTGC |
| | | | TGACGCTGGTAACCGTTATGTCTTCGGCGGAGGCACC |
| | | | AAGCTGACGGTCCTAGGTGGCCAGGCCGGCCAGACAA |
| | | | CGACACCTGCTCCCAGACCGCCTACTCCCGCCCCAAC |
| | | | CATTGCATCTCAGCCACTCTCTCTGAGACCCGAAGCGT |
| | | | GTAGACCTGCGGCCGGGGGCGCTGTCCACACAAGAG |
| | | | GCTTAGACTTCGCCTGCGATATCTATATCTGGGCCCCA |
| | | | CTCGCAGGCACTTGTGGAGTGCTGCTGCTTTCACTCGT |
| | | | GATAACCCTGTACTGCAAAAGGGGGAGAAAGAAGCTG |
| | | | CTGTATATTTTTAAACAACCATTTATGAGACCTGTTCAG |
| | | | ACTACCCAGGAAGAAGACGGTTGTAGTTGCAGATTCCC |
| | | | CGAGGAGGAAGAAGGAGGTTGCGAGTTGAGAGTAAAG |
| | | | TTCAGCAGATCCGCAGATGCCCCTGCTTACCAGCAGG |
| | | | GTCAAAACCAGCTTTACAACGAGCTGAATTTAGGTAGA |
| | | | AGAGAGGAATATGACGTGTTGGATAAAAGAAGAGGAA |
| | | | GAGACCCGGAAATGGGCGGCAAGCCTCGAAGAAAAAA |
| | | | TCCCCAAGAGGGACTCTACAATGAGCTGCAGAAGGAC |
| | | | AAAATGGCTGAAGCCTACAGCGAGATCGGCATGAAGG |
| | | | GAGAAAGACGCAGAGGGAAAGGGCATGATGGGCTTTA |
| | | | TCAGGGCTTGTCCACCGCTACAAAGGATACTTATGACG |
| | | | CACTACACATGCAGGCCCTGCCACCCCGT |
| 161 | Protein | CDRH1 of anti CD30 | YYDMS |
| | | antibody (T1-141) | |
| 162 | Protein | CDRH2 of anti CD30 | GIYGSGSTYYADSVKG |
| | | antibody (T1-141) | |
| 163 | Protein | CDRH3 of anti CD30 | YSYYDSYSDYFDY |
| | | antibody (T1-141) | |
| 164 | Protein | CDRL1 of anti CD30 | SGSSSNIGSNYVY |
| | | antibody (T1-141) | |
| 165 | Protein | CDRL2 of anti CD30 | RNNQRPS |
| | | antibody (T1-141) | |
| 166 | Protein | CDRL3 of anti CD30 | AAYSYGY |
| | | antibody (T1-141) | |
| 167 | Protein | Heavy chain variable | EVQLLESGGGLVQPGGSLRLSCAASGFTFSYYDMSWVR |
| | | domain of anti CD30 | QAPGKGLEWVSGIYGSGSTYYADSVKGRFTISRDNSKNT |
| | | antibody | LYLQMNSLRAEDTAVYYCARYSYYDSYSDYFDYWGQGT |
| | | (T1-141) | LVTVSS |
| 168 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAAYSYGYYVFGGGTKLTVLG |
| | | (T1-141) | |
| 169 | Protein | scFv of anti CD30 | EVQLLESGGGLVQPGGSLRLSCAASGFTFSYYDMSWVR |
| | | antibody | QAPGKGLEWVSGIYGSGSTYYADSVKGRFTISRDNSKNT |
| | | (T1-141) | LYLQMNSLRAEDTAVYYCARYSYYDSYSDYFDYWGQGT |
| | | | LVTVSSGGGGSGGGGSGGGGSQSVLTQPPSASGTPGQ |
| | | | RVTISCSGSSSNIGSNYVYWYQQLPGTAPKLLIYRNNQRP |
| | | | SGVPDRFSGSKSGTSASLAISGLRSEDEADYYCAAYSYG |
| | | | YYVFGGGTKLTVLG |
| 170 | Protein | scFv CAR of anti | EVQLLESGGGLVQPGGSLRLSCAASGFTFSYYDMSWVR |
| | | CD30 antibody | QAPGKGLEWVSGIYGSGSTYYADSVKGRFTISRDNSKNT |
| | | (T1-141) | LYLQMNSLRAEDTAVYYCARYSYYDSYSDYFDYWGQGT |
| | | | LVTVSSGGGGSGGGGSGGGGSQSVLTQPPSASGTPGQ |
| | | | RVTISCSGSSSNIGSNYVYWYQQLPGTAPKLLIYRNNQRP |
| | | | SGVPDRFSGSKSGTSASLAISGLRSEDEADYYCAAYSYG |
| | | | YYVFGGGTKLTVLGGQAGQTTTPAPRPPTPAPTIASQPL |
| | | | SLRPEACRPAAGGAVHTRGLDFACDIYIWAPLAGTCGVLL |
| | | | LSLVITLYCKRGRKKLLYIFKQPFMRPVQTTQEEDGCSCR |
| | | | FPEEEEGGCELRVKFSRSADAPAYQQGQNQLYNELNLG |
| | | | RREEYDVLDKRRGRDPEMGGKPRRKNPQEGLYNELQK |
| | | | DKMAEAYSEIGMKGERRRGKGHDGLYQGLSTATKDTYD |
| | | | ALHMQALPPR |
| 171 | DNA | Nucleotide sequence | TACTATGATATGAGC |
| | | for encoding CDRH1 | |
| | | of anti CD30 | |
| | | antibody (T1-141) | |
| 172 | DNA | Nucleotide sequence | GGTATCTACGGTTCTGGTTCTACGTATTACGCTGATTC |
| | | for encoding CDRH2 | TGTAAAAGGT |
| | | of anti CD30 | |
| | | antibody (T1-141) | |
| 173 | DNA | Nucleotide sequence | TACTCTTACTACGACTCTTACTCTGACTACTTCGACTAC |
| | | for encoding CDRH3 | |
| | | of anti CD30 | |
| | | antibody (T1-141) | |
| 174 | DNA | Nucleotide sequence | AGTGGCTCTTCATCTAATATTGGCTCTAATTATGTCTAC |
| | | for encoding CDRL1 | |
| | | of anti CD30 | |
| | | antibody (T1-141) | |
| 175 | DNA | Nucleotide sequence | AGAAATAACCAGCGGCCAAGC |
| | | for encoding CDRL2 | |
| | | of anti CD30 | |
| | | antibody (T1-141) | |
| 176 | DNA | Nucleotide sequence | GCTGCTTACTCTTACGGTTAC |
| | | for encoding CDRL3 | |
| | | of anti CD30 | |
| | | antibody (T1-141) | |
| 177 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTACTATGATATGAGCTGGGTCC |
| | | of anti CD30 | GCCAGGCTCCAGGGAAGGGGCTGGAGTGGGTCTCAG |
| | | antibody (T1-141) | GTATCTACGGTTCTGGTTCTACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTCTTA |
| | | | CTACGACTCTTACTCTGACTACTTCGACTACTGGGGCC |
| | | | AGGGTACACTGGTCACCGTGAGCTCA |
| 178 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 antibody | GCAGCTCCCAGGAACGGCCCCCAAACTCCTCATCTAT |
| | | (T1-141) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACTCTTACGGTTACTATGTCTTCGG |
| | | | CGGAGGCACCAAGCTGACGGTCCTAGGT |
| 179 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTACTATGATATGAGCTGGGTCC |
| | | (T1-141) | GCCAGGCTCCAGGGAAGGGGCTGGAGTGGGTCTCAG |
| | | | GTATCTACGGTTCTGGTTCTACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTCTTA |
| | | | CTACGACTCTTACTCTGACTACTTCGACTACTGGGGCC |
| | | | AGGGTACACTGGTCACCGTGAGCTCAGGTGGAGGCG |
| | | | GTTCAGGCGGAGGTGGATCCGGCGGTGGCGGATCGC |
| | | | AGTCTGTGCTGACTCAGCCACCCTCAGCGTCTGGGAC |
| | | | CCCCGGGCAGAGGGTCACCATCTCTTGTAGTGGCTCT |
| | | | TCATCTAATATTGGCTCTAATTATGTCTACTGGTACCAG |
| | | | CAGCTCCCAGGAACGGCCCCCAAACTCCTCATCTATA |
| | | | GAAATAACCAGCGGCCAAGCGGGGTCCCTGACCGATT |
| | | | CTCTGGCTCCAAGTCTGGCACCTCAGCCTCCCTGGCC |
| | | | ATCAGTGGGCTCCGGTCCGAGGATGAGGCCGATTATT |
| | | | ACTGTGCTGCTTACTCTTACGGTTACTATGTCTTCGGC |
| | | | GGAGGCACCAAGCTGACGGTCCTAGGT |
| 180 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTACTATGATATGAGCTGGGTCC |
| | | antibody | GCCAGGCTCCAGGGAAGGGGCTGGAGTGGGTCTCAG |
| | | (T1-141) | GTATCTACGGTTCTGGTTCTACGTATTACGCTGATTCT |
| | | | GTAAAAGGTCGGTTCACCATCTCCAGAGACAATTCCAA |
| | | | GAACACGCTGTATCTGCAAATGAACAGCCTGAGAGCC |
| | | | GAGGACACGGCCGTGTATTACTGTGCGCGTTACTCTTA |
| | | | CTACGACTCTTACTCTGACTACTTCGACTACTGGGGCC |
| | | | AGGGTACACTGGTCACCGTGAGCTCAGGTGGAGGCG |
| | | | GTTCAGGCGGAGGTGGATCCGGCGGTGGCGGATCGC |
| | | | AGTCTGTGCTGACTCAGCCACCCTCAGCGTCTGGGAC |
| | | | CCCCGGGCAGAGGGTCACCATCTCTTGTAGTGGCTCT |
| | | | TCATCTAATATTGGCTCTAATTATGTCTACTGGTACCAG |
| | | | CAGCTCCCAGGAACGGCCCCCAAACTCCTCATCTATA |
| | | | GAAATAACCAGCGGCCAAGCGGGGTCCCTGACCGATT |
| | | | CTCTGGCTCCAAGTCTGGCACCTCAGCCTCCCTGGCC |
| | | | ATCAGTGGGCTCCGGTCCGAGGATGAGGCCGATTATT |
| | | | ACTGTGCTGCTTACTCTTACGGTTACTATGTCTTCGGC |
| | | | GGAGGCACCAAGCTGACGGTCCTAGGTGGCCAGGCC |
| | | | GGCCAGACAACGACACCTGCTCCCAGACCGCCTACTC |
| | | | CCGCCCCAACCATTGCATCTCAGCCACTCTCTCTGAGA |
| | | | CCCGAAGCGTGTAGACCTGCGGCCGGGGGCGCTGTC |
| | | | CACACAAGAGGCTTAGACTTCGCCTGCGATATCTATAT |
| | | | CTGGGCCCCACTCGCAGGCACTTGTGGAGTGCTGCTG |
| | | | CTTTCACTCGTGATAACCCTGTACTGCAAAAGGGGGAG |
| | | | AAAGAAGCTGCTGTATATTTTTAAACAACCATTTATGAG |
| | | | ACCTGTTCAGACTACCCAGGAAGAAGACGGTTGTAGTT |
| | | | GCAGATTCCCCGAGGAGGAAGAAGGAGGTTGCGAGTT |
| | | | GAGAGTAAAGTTCAGCAGATCCGCAGATGCCCCTGCT |
| | | | TACCAGCAGGGTCAAAACCAGCTTTACAACGAGCTGAA |
| | | | TTTAGGTAGAAGAGAGGAATATGACGTGTTGGATAAAA |
| | | | GAAGAGGAAGAGACCCGGAAATGGGCGGCAAGCCTC |
| | | | GAAGAAAAAATCCCCAAGAGGGACTCTACAATGAGCTG |
| | | | CAGAAGGACAAAATGGCTGAAGCCTACAGCGAGATCG |
| | | | GCATGAAGGGAGAAAGACGCAGAGGGAAAGGGCATG |
| | | | ATGGGCTTTATCAGGGCTTGTCCACCGCTACAAAGGAT |
| | | | ACTTATGACGCACTACACATGCAGGCCCTGCCACCCC |
| | | | GT |
| 181 | Protein | CDRH1 of anti CD30 | SYGMS |
| | | antibody (T1-159) | |
| 182 | Protein | CDRH2 of anti CD30 | SIYYYSGGTYYADSVKG |
| | | antibody (T1-159) | |
| 183 | Protein | CDRH3 of anti CD30 | NDYRNNSDFDY |
| | | antibody (T1-159) | |
| 184 | Protein | CDRL1 of anti CD30 | SGSSSNIGSNYVY |
| | | antibody (T1-159) | |
| 185 | Protein | CDRL2 of anti CD30 | RNNQRPS |
| | | antibody (T1-159) | |
| 186 | Protein | CDRL3 of anti CD30 | AAYYPYY |
| | | antibody (T1-159) | |
| 187 | Protein | Heavy chain variable | EVQLLESGGGLVQPGGSLRLSCAASGFTFSSYGMSWVR |
| | | domain of anti CD30 | QAPGKGLEWVSSIYYYSGGTYYADSVKGRFTISRDNSKN |
| | | antibody | TLYLQMNSLRAEDTAVYYCARNDYRNNSDFDYWGQGTL |
| | | (T1-159) | VTVSS |
| 188 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody | RSEDEADYYCAAYYPYYYVFGGGTKLTVLG |
| | | (T1-159) | |
| 189 | Protein | scFv of anti CD30 | EVQLLESGGGLVQPGGSLRLSCAASGFTFSSYGMSWVR |
| | | antibody | QAPGKGLEWVSSIYYYSGGTYYADSVKGRFTISRDNSKN |
| | | (T1-159) | TLYLQMNSLRAEDTAVYYCARNDYRNNSDFDYWGQGTL |
| | | | VTVSSGGGGSGGGGSGGGGSQSVLTQPPSASGTPGQR |
| | | | VTISCSGSSSNIGSNYVYWYQQLPGTAPKLLIYRNNQRPS |
| | | | GVPDRFSGSKSGTSASLAISGLRSEDEADYYCAAYYPYY |
| | | | YVFGGGTKLTVLG |
| 190 | Protein | scFv CAR of anti | EVQLLESGGGLVQPGGSLRLSCAASGFTFSSYGMSWVR |
| | | CD30 antibody | QAPGKGLEWVSSIYYYSGGTYYADSVKGRFTISRDNSKN |
| | | (T1-159) | TLYLQMNSLRAEDTAVYYCARNDYRNNSDFDYWGQGTL |
| | | | VTVSSGGGGSGGGGSGGGGSQSVLTQPPSASGTPGQR |
| | | | VTISCSGSSSNIGSNYVYWYQQLPGTAPKLLIYRNNQRPS |
| | | | GVPDRFSGSKSGTSASLAISGLRSEDEADYYCAAYYPYY |
| | | | YVFGGGTKLTVLGGQAGQTTTPAPRPPTPAPTIASQPLSL |
| | | | RPEACRPAAGGAVHTRGLDFACDIYIWAPLAGTCGVLLLS |
| | | | LVITLYCKRGRKKLLYIFKQPFMRPVQTTQEEDGCSCRFP |
| | | | EEEEGGCELRVKFSRSADAPAYQQGQNQLYNELNLGRR |
| | | | EEYDVLDKRRGRDPEMGGKPRRKNPQEGLYNELQKDK |
| | | | MAEAYSEIGMKGERRRGKGHDGLYQGLSTATKDTYDAL |
| | | | HMQALPPR |
| 191 | DNA | Nucleotide sequence | TCTTATGGTATGAGC |
| | | for encoding CDRH1 | |
| | | of anti CD30 | |
| | | antibody (T1-159) | |
| 192 | DNA | Nucleotide sequence | TCTATCTACTACTACTCTGGTGGTACGTATTACGCTGAT |
| | | for encoding CDRH2 | TCTGTAAAAGGT |
| | | of anti CD30 | |
| | | antibody (T1-159) | |
| 193 | DNA | Nucleotide sequence | AACGACTACCGTAACAACTCTGACTTCGACTAC |
| | | for encoding CDRH3 | |
| | | of anti CD30 | |
| | | antibody (T1-159) | |
| 194 | DNA | Nucleotide sequence | AGTGGCTCTTCATCTAATATTGGCTCTAATTATGTCTAC |
| | | for encoding CDRL1 | |
| | | of anti CD30 | |
| | | antibody (T1-159) | |
| 195 | DNA | Nucleotide sequence | AGAAATAACCAGCGGCCAAGC |
| | | for encoding CDRL2 | |
| | | of anti CD30 | |
| | | antibody (T1-159) | |
| 196 | DNA | Nucleotide sequence | GCTGCTTACTACCCGTACTAC |
| | | for encoding CDRL3 | |
| | | of anti CD30 | |
| | | antibody (T1-159) | |
| 197 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTCTTATGGTATGAGCTGGGTCC |
| | | of anti CD30 | GCCAGGCTCCAGGGAAGGGGCTGGAGTGGGTCTCAT |
| | | antibody (T1-159) | CTATCTACTACTACTCTGGTGGTACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTAACGAC |
| | | | TACCGTAACAACTCTGACTTCGACTACTGGGGCCAGG |
| | | | GTACACTGGTCACCGTGAGCTCA |
| 198 | DNA | Nucleotide sequence | CAGTCTGTGCTGACTCAGCCACCCTCAGCGTCTGGGA |
| | | for encoding light | CCCCCGGGCAGAGGGTCACCATCTCTTGTAGTGGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T1-159) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGCTTACTACCCGTACTACTATGTCTTCGG |
| | | | CGGAGGCACCAAGCTGACGGTCCTAGGT |
| 199 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTCTTATGGTATGAGCTGGGTCC |
| | | (T1-159) | GCCAGGCTCCAGGGAAGGGGCTGGAGTGGGTCTCAT |
| | | | CTATCTACTACTACTCTGGTGGTACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTAACGAC |
| | | | TACCGTAACAACTCTGACTTCGACTACTGGGGCCAGG |
| | | | GTACACTGGTCACCGTGAGCTCAGGTGGAGGCGGTTC |
| | | | AGGCGGAGGTGGATCCGGCGGTGGCGGATCGCAGTC |
| | | | TGTGCTGACTCAGCCACCCTCAGCGTCTGGGACCCCC |
| | | | GGGCAGAGGGTCACCATCTCTTGTAGTGGCTCTTCATC |
| | | | TAATATTGGCTCTAATTATGTCTACTGGTACCAGCAGCT |
| | | | CCCAGGAACGGCCCCCAAACTCCTCATCTATAGAAATA |
| | | | ACCAGCGGCCAAGCGGGGTCCCTGACCGATTCTCTGG |
| | | | CTCCAAGTCTGGCACCTCAGCCTCCCTGGCCATCAGT |
| | | | GGGCTCCGGTCCGAGGATGAGGCCGATTATTACTGTG |
| | | | CTGCTTACTACCCGTACTACTATGTCTTCGGCGGAGGC |
| | | | ACCAAGCTGACGGTCCTAGGT |
| 200 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTCTTATGGTATGAGCTGGGTCC |
| | | antibody | GCCAGGCTCCAGGGAAGGGGCTGGAGTGGGTCTCAT |
| | | (T1-159) | CTATCTACTACTACTCTGGTGGTACGTATTACGCTGATT |
| | | | CTGTAAAAGGTCGGTTCACCATCTCCAGAGACAATTCC |
| | | | AAGAACACGCTGTATCTGCAAATGAACAGCCTGAGAGC |
| | | | CGAGGACACGGCCGTGTATTACTGTGCGCGTAACGAC |
| | | | TACCGTAACAACTCTGACTTCGACTACTGGGGCCAGG |
| | | | GTACACTGGTCACCGTGAGCTCAGGTGGAGGCGGTTC |
| | | | AGGCGGAGGTGGATCCGGCGGTGGCGGATCGCAGTC |
| | | | TGTGCTGACTCAGCCACCCTCAGCGTCTGGGACCCCC |
| | | | GGGCAGAGGGTCACCATCTCTTGTAGTGGCTCTTCATC |
| | | | TAATATTGGCTCTAATTATGTCTACTGGTACCAGCAGCT |
| | | | CCCAGGAACGGCCCCCAAACTCCTCATCTATAGAAATA |
| | | | ACCAGCGGCCAAGCGGGGTCCCTGACCGATTCTCTGG |
| | | | CTCCAAGTCTGGCACCTCAGCCTCCCTGGCCATCAGT |
| | | | GGGCTCCGGTCCGAGGATGAGGCCGATTATTACTGTG |
| | | | CTGCTTACTACCCGTACTACTATGTCTTCGGCGGAGGC |
| | | | ACCAAGCTGACGGTCCTAGGTGGCCAGGCCGGCCAG |
| | | | ACAACGACACCTGCTCCCAGACCGCCTACTCCCGCCC |
| | | | CAACCATTGCATCTCAGCCACTCTCTCTGAGACCCGAA |
| | | | GCGTGTAGACCTGCGGCCGGGGGCGCTGTCCACACA |
| | | | AGAGGCTTAGACTTCGCCTGCGATATCTATATCTGGGC |
| | | | CCCACTCGCAGGCACTTGTGGAGTGCTGCTGCTTTCA |
| | | | CTCGTGATAACCCTGTACTGCAAAAGGGGGAGAAAGA |
| | | | AGCTGCTGTATATTTTTAAACAACCATTTATGAGACCTG |
| | | | TTCAGACTACCCAGGAAGAAGACGGTTGTAGTTGCAGA |
| | | | TTCCCCGAGGAGGAAGAAGGAGGTTGCGAGTTGAGAG |
| | | | TAAAGTTCAGCAGATCCGCAGATGCCCCTGCTTACCAG |
| | | | CAGGGTCAAAACCAGCTTTACAACGAGCTGAATTTAGG |
| | | | TAGAAGAGAGGAATATGACGTGTTGGATAAAAGAAGAG |
| | | | GAAGAGACCCGGAAATGGGGGGCAAGCCTCGAAGAAA |
| | | | AAATCCCCAAGAGGGACTCTACAATGAGCTGCAGAAG |
| | | | GACAAAATGGCTGAAGCCTACAGCGAGATCGGCATGA |
| | | | AGGGAGAAAGACGCAGAGGGAAAGGGCATGATGGGC |
| | | | TTTATCAGGGCTTGTCCACCGCTACAAAGGATACTTAT |
| | | | GACGCACTACACATGCAGGCCCTGCCACCCCGT |
| 201 | Protein | CDRH1 of anti CD30 | SYHMS |
| | | antibody (T2-2) | |
| 202 | Protein | CDRH2 of anti CD30 | GIGYGHGGTYYADSVKG |
| | | antibody (T2-2) | |
| 203 | Protein | CDRH3 of anti CD30 | YDYDYSFDY |
| | | antibody (T2-2) | |
| 204 | Protein | CDRL1 of anti CD30 | SGSSSNIGSNYVY |
| | | antibody (T2-2) | |
| 205 | Protein | CDRL2 of anti CD30 | RNNQRPS |
| | | antibody (T2-2) | |
| 206 | Protein | CDRL3 of anti CD30 | AVSSPYS |
| | | antibody (T2-2) | |
| 207 | Protein | Heavy chain variable | EVQLLESGGGLVQPGGSLRLSCAASGFTFSSYHMSWVR |
| | | domain of anti CD30 | QAPGKGLEWVSGIGYGHGGTYYADSVKGRFTISRDNSK |
| | | antibody (T2-2) | NTLYLQMNSLRAEDTAVYYCARYDYDYSFDYWGQGTLV |
| | | | TVSS |
| 208 | Protein | Light chain variable | QSVLTQPPSASGTPGQRVTISCSGSSSNIGSNYVYWYQQ |
| | | domain of anti CD30 | LPGTAPKLLIYRNNQRPSGVPDRFSGSKSGTSASLAISGL |
| | | antibody (T2-2) | RSEDEADYYCAVSSPYSYVFGGGTKLTVLG |
| 209 | Protein | scFv of anti CD30 | EVQLLESGGGLVQPGGSLRLSCAASGFTFSSYHMSWVR |
| | | antibody | QAPGKGLEWVSGIGYGHGGTYYADSVKGRFTISRDNSK |
| | | (T2-2) | NTLYLQMNSLRAEDTAVYYCARYDYDYSFDYWGQGTLV |
| | | | TVSSGGGGSGGGGSGGGGSQSVLTQPPSASGTPGQRV |
| | | | TISCSGSSSNIGSNYVYWYQQLPGTAPKLLIYRNNQRPSG |
| | | | VPDRFSGSKSGTSASLAISGLRSEDEADYYCAVSSPYSY |
| | | | VFGGGTKLTVLG |
| 210 | Protein | scFv CAR of anti | EVQLLESGGGLVQPGGSLRLSCAASGFTFSSYHMSWVR |
| | | CD30 antibody | QAPGKGLEWVSGIGYGHGGTYYADSVKGRFTISRDNSK |
| | | (T2-2) | NTLYLQMNSLRAEDTAVYYCARYDYDYSFDYWGQGTLV |
| | | | TVSSGGGGSGGGGSGGGGSQSVLTQPPSASGTPGQRV |
| | | | TISCSGSSSNIGSNYVYWYQQLPGTAPKLLIYRNNQRPSG |
| | | | VPDRFSGSKSGTSASLAISGLRSEDEADYYCAVSSPYSY |
| | | | VFGGGTKLTVLGGQAGQTTTPAPRPPTPAPTIASQPLSLR |
| | | | PEACRPAAGGAVHTRGLDFACDIYIWAPLAGTCGVLLLSL |
| | | | VITLYCKRGRKKLLYIFKQPFMRPVQTTQEEDGCSCRFPE |
| | | | EEEGGCELRVKFSRSADAPAYQQGQNQLYNELNLGRRE |
| | | | EYDVLDKRRGRDPEMGGKPRRKNPQEGLYNELQKDKM |
| | | | AEAYSEIGMKGERRRGKGHDGLYQGLSTATKDTYDALH |
| | | | MQALPPR |
| 211 | DNA | Nucleotide sequence | TCTTATCACATGAGC |
| | | for encoding CDRH1 | |
| | | of anti CD30 | |
| | | antibody (T2-2) | |
| 212 | DNA | Nucleotide sequence | GGTATCGGTTACGGTCACGGTGGTACGTATTACGCTG |
| | | for encoding CDRH2 | ATTCTGTAAAAGGT |
| | | of anti CD30 | |
| | | antibody (T2-2) | |
| 213 | DNA | Nucleotide sequence | TACGACTACGACTACTCTTTCGACTAC |
| | | for encoding CDRH3 | |
| | | of anti CD30 | |
| | | antibody (T2-2) | |
| 214 | DNA | Nucleotide sequence | TCCTGCTCTTCATCTAATATTGGCTCTAATTATGTCTAC |
| | | for encoding CDRL1 | |
| | | of anti CD30 | |
| | | antibody (T2-2) | |
| 215 | DNA | Nucleotide sequence | AGAAATAACCAGCGGCCAAGC |
| | | for encoding CDRL2 | |
| | | of anti CD30 | |
| | | antibody (T2-2) | |
| 216 | DNA | Nucleotide sequence | GCTGTTTCTTCTCCGTACTCT |
| | | for encoding CDRL3 | |
| | | of anti CD30 | |
| | | antibody (T2-2) | |
| 217 | DNA | Nucleotide sequence | GAGGTGCAGCTGTTGGAGTCTGGGGGAGGCTTGGTAC |
| | | for encoding heavy | AGCCTGGGGGGTCCCTGAGACTCTCCTGTGCAGCCTC |
| | | chain variable domain | TGGATTCACCTTTAGCTCTTATCACATGAGCTGGGTCC |
| | | of anti CD30 | GCCAGGCTCCAGGGAAGGGGCTGGAGTGGGTCTCAG |
| | | antibody (T2-2) | GTATCGGTTACGGTCACGGTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTTACGA |
| | | | CTACGACTACTCTTTCGACTACTGGGGCCAGGGTACAC |
| | | | TGGTCACCGTGAGCTCA |
| 218 | DNA | Nucleotide sequence | CAGTCTGTGCTGACTCAGCCACCCTCAGCGTCTGGGA |
| | | for encoding light | CCCCCGGGCAGAGGGTCACCATCTCTTGTTCCTGCTC |
| | | chain variable domain | TTCATCTAATATTGGCTCTAATTATGTCTACTGGTACCA |
| | | of anti CD30 | GCAGCTCCCAGGAACGGCCCCCAAACTCCTCATCTAT |
| | | antibody (T2-2) | AGAAATAACCAGCGGCCAAGCGGGGTCCCTGACCGAT |
| | | | TCTCTGGCTCCAAGTCTGGCACCTCAGCCTCCCTGGC |
| | | | CATCAGTGGGCTCCGGTCCGAGGATGAGGCCGATTAT |
| | | | TACTGTGCTGTTTCTTCTCCGTACTCTTATGTCTTCGGC |
| | | | GGAGGCACCAAGCTGACGGTCCTAGGT |
| 219 | DNA | Nucleotide sequence | GAGGTGCAGCTGTTGGAGTCTGGGGGAGGCTTGGTAC |
| | | for encoding scFv of | AGCCTGGGGGGTCCCTGAGACTCTCCTGTGCAGCCTC |
| | | anti CD30 antibody | TGGATTCACCTTTAGCTCTTATCACATGAGCTGGGTCC |
| | | (T2-2) | GCCAGGCTCCAGGGAAGGGGCTGGAGTGGGTCTCAG |
| | | | GTATCGGTTACGGTCACGGTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTTACGA |
| | | | CTACGACTACTCTTTCGACTACTGGGGCCAGGGTACAC |
| | | | TGGTCACCGTGAGCTCAGGTGGAGGCGGTTCAGGCG |
| | | | GAGGTGGATCCGGCGGTGGCGGATCGCAGTCTGTGC |
| | | | TGACTCAGCCACCCTCAGCGTCTGGGACCCCCGGGCA |
| | | | GAGGGTCACCATCTCTTGTAGTGGCTCTTCATCTAATA |
| | | | TTGGCTCTAATTATGTCTACTGGTACCAGCAGCTCCCA |
| | | | GGAACGGCCCCCAAACTCCTCATCTATAGAAATAACCA |
| | | | GCGGCCAAGCGGGGTCCCTGACCGATTCTCTGGCTCC |
| | | | AAGTCTGGCACCTCAGCCTCCCTGGCCATCAGTGGGC |
| | | | TCCGGTCCGAGGATGAGGCCGATTATTACTGTGCTGTT |
| | | | TCTTCTCCGTACTCTTATGTCTTCGGCGGAGGCACCAA |
| | | | GCTGACGGTCCTAGGT |
| 220 | DNA | Nucleotide sequence | GAGGTGCAGCTGTTGGAGTCTGGGGGAGGCTTGGTAC |
| | | for encoding scFv | AGCCTGGGGGGTCCCTGAGACTCTCCTGTGCAGCCTC |
| | | CAR of anti CD30 | TGGATTCACCTTTAGCTCTTATCACATGAGCTGGGTCC |
| | | antibody | GCCAGGCTCCAGGGAAGGGGCTGGAGTGGGTCTCAG |
| | | (T2-2) | GTATCGGTTACGGTCACGGTGGTACGTATTACGCTGAT |
| | | | TCTGTAAAAGGTCGGTTCACCATCTCCAGAGACAATTC |
| | | | CAAGAACACGCTGTATCTGCAAATGAACAGCCTGAGAG |
| | | | CCGAGGACACGGCCGTGTATTACTGTGCGCGTTACGA |
| | | | CTACGACTACTCTTTCGACTACTGGGGCCAGGGTACAC |
| | | | TGGTCACCGTGAGCTCAGGTGGAGGCGGTTCAGGCG |
| | | | GAGGTGGATCCGGCGGTGGCGGATCGCAGTCTGTGC |
| | | | TGACTCAGCCACCCTCAGCGTCTGGGACCCCCGGGCA |
| | | | GAGGGTCACCATCTCTTGTAGTGGCTCTTCATCTAATA |
| | | | TTGGCTCTAATTATGTCTACTGGTACCAGCAGCTCCCA |
| | | | GGAACGGCCCCCAAACTCCTCATCTATAGAAATAACCA |
| | | | GCGGCCAAGCGGGGTCCCTGACCGATTCTCTGGCTCC |
| | | | AAGTCTGGCACCTCAGCCTCCCTGGCCATCAGTGGGC |
| | | | TCCGGTCCGAGGATGAGGCCGATTATTACTGTGCTGTT |
| | | | TCTTCTCCGTACTCTTATGTCTTCGGCGGAGGCACCAA |
| | | | GCTGACGGTCCTAGGTGGCCAGGCCGGCCAGACAAC |
| | | | GACACCTGCTCCCAGACCGCCTACTCCCGCCCCAACC |
| | | | ATTGCATCTCAGCCACTCTCTCTGAGACCCGAAGCGTG |
| | | | TAGACCTGCGGCCGGGGGCGCTGTCCACACAAGAGG |
| | | | CTTAGACTTCGCCTGCGATATCTATATCTGGGCCCCAC |
| | | | TCGCAGGCACTTGTGGAGTGCTGCTGCTTTCACTCGT |
| | | | GATAACCCTGTACTGCAAAAGGGGGAGAAAGAAGCTG |
| | | | CTGTATATTTTTAAACAACCATTTATGAGACCTGTTCAG |
| | | | ACTACCCAGGAAGAAGACGGTTGTAGTTGCAGATTCCC |
| | | | CGAGGAGGAAGAAGGAGGTTGCGAGTTGAGAGTAAAG |
| | | | TTCAGCAGATCCGCAGATGCCCCTGCTTACCAGCAGG |
| | | | GTCAAAACCAGCTTTACAACGAGCTGAATTTAGGTAGA |
| | | | AGAGAGGAATATGACGTGTTGGATAAAAGAAGAGGAA |
| | | | GAGACCCGGAAATGGGCGGCAAGCCTCGAAGAAAAAA |
| | | | TCCCCAAGAGGGACTCTACAATGAGCTGCAGAAGGAC |
| | | | AAAATGGCTGAAGCCTACAGCGAGATCGGCATGAAGG |
| | | | GAGAAAGACGCAGAGGGAAAGGGCATGATGGGCTTTA |
| | | | TCAGGGCTTGTCCACCGCTACAAAGGATACTTATGACG |
| | | | CACTACACATGCAGGCCCTGCCACCCCGT |
| 221 | DNA | Nucleotide sequence | TGTGAGGCTCCGGTGCCCGTCAGTGGGCAGAGCGCA |
| | | for encoding EF-1 | CATCGCCCACAGTCCCCGAGAAGTTGGGGGGAGGGG |
| | | alpha promoter | TCGGCAATTGAACCGGTGCCTAGAGAAGGTGGCGCGG |
| | | | GGTAAACTGGGAAAGTGATGTCGTGTACTGGCTCCGC |
| | | | CTTTTTCCCGAGGGTGGGGGAGAACCGTATATAAGTG |
| | | | CAGTAGTCGCCGTGAACGTTCTTTTTCGCAACGGGTTT |
| | | | GCCGCCAGAACACAGGTAAGTGCCGTGTGTGGTTCCC |
| | | | GCGGGCCTGGCCTCTTTACGGGTTATGGCCCTTGCGT |
| | | | GCCTTGAATTACTTCCACCTGGCTGCAGTACGTGATTC |
| | | | TTGATCCCGAGCTTCGGGTTGGAAGTGGGTGGGAGAG |
| | | | TTCGAGGCCTTGCGCTTAAGGAGCCCCTTCGCCTCGT |
| | | | GCTTGAGTTGAGGCCTGGCCTGGGCGCTGGGGCCGC |
| | | | CGCGTGCGAATCTGGTGGCACCTTCGCGCCTGTCTCG |
| | | | CTGCTTTCGATAAGTCTCTAGCCATTTAAAATTTTTGAT |
| | | | GACCTGCTGCGACGCTTTTTTTCTGGCAAGATAGTCTT |
| | | | GTAAATGCGGGCCAAGATCTGCACACTGGTATTTCGGT |
| | | | TTTTGGGGCCGCGGGGGGCGACGGGGCCCGTGCGTC |
| | | | CCAGCGCACATGTTCGGCGAGGCGGGGCCTGCGAGC |
| | | | GCGGCCACCGAGAATCGGACGGGGGTAGTCTCAAGCT |
| | | | GGCCGGCCTGCTCTGGTGCCTGGCCTCGCGCCGCCG |
| | | | TGTATCGCCCCGCCCTGGGCGGCAAGGCTGGCCCGG |
| | | | TCGGCACCAGTTGCGTGAGCGGAAAGATGGCCGCTTC |
| | | | CCGGCCCTGCTGCAGGGAGCTCAAAATGGAGGACGC |
| | | | GGCGCTCGGGAGAGCGGGCGGGTGAGTCACCCACAC |
| | | | AAAGGAAAAGGGCCTTTCCGTCCTCAGCCGTCGCTTC |
| | | | ATGTGACTCCACGGAGTACCGGGCGCCGTCCAGGCAC |
| | | | CTCGATTAGTTCTCGAGCTTTTGGAGTACGTCGTCTTT |
| | | | AGGTTGGGGGGAGGGGTTTTATGCGATGGAGTTTCCC |
| | | | CACACTGAGTGGGTGGAGACTGAAGTTAGGCCAGCTT |
| | | | GGCACTTGATGTAATTCTCCTTGGAATTTGCCCTTTTTG |
| | | | AGTTTGGATCTTGGTTCATTCTCAAGCCTCAGACAGTG |
| | | | GTTCAAAGTTTTTTTCTTCCATTTCAGGTGTCGTGA |
| 222 | DNA | Nucleotide sequence | GGCCAGGCCGGCCAGACAACGACACCTGCTCCCAGA |
| | | for encoding CAR | CCGCCTACTCCCGCCCCAACCATTGCATCTCAGCCACT |
| | | construct | CTCTCTGAGACCCGAAGCGTGTAGACCTGCGGCCGGG |
| | | | GGCGCTGTCCACACAAGAGGCTTAGACTTCGCCTGCG |
| | | | ATATCTATATCTGGGCCCCACTCGCAGGCACTTGTGGA |
| | | | GTGCTGCTGCTTTCACTCGTGATAACCCTGTACTGCAA |
| | | | AAGGGGGAGAAAGAAGCTGCTGTATATTTTTAAACAAC |
| | | | CATTTATGAGACCTGTTCAGACTACCCAGGAAGAAGAC |
| | | | GGTTGTAGTTGCAGATTCCCCGAGGAGGAAGAAGGAG |
| | | | GTTGCGAGTTGAGAGTAAAGTTCAGCAGATCCGCAGAT |
| | | | GCCCCTGCTTACCAGCAGGGTCAAAACCAGCTTTACAA |
| | | | CGAGCTGAATTTAGGTAGAAGAGAGGAATATGACGTGT |
| | | | TGGATAAAAGAAGAGGAAGAGACCCGGAAATGGGGGG |
| | | | CAAGCCTCGAAGAAAAAATCCCCAAGAGGGACTCTACA |
| | | | ATGAGCTGCAGAAGGACAAAATGGCTGAAGCCTACAG |
| | | | CGAGATCGGCATGAAGGGAGAAAGACGCAGAGGGAAA |
| | | | GGGCATGATGGGCTTTATCAGGGCTTGTCCACCGCTA |
| | | | CAAAGGATACTTATGACGCACTACACATGCAGGCCCTG |
| | | | CCACCCCGT |
| 223 | Protein | CD30 extracellular | FPQDRPFEDTCHGNPSHYYDKAVRRCCYRCPMGLFPTQ |
| | | region (CD30 domain | QCPQRPTDCRKQCEPDYYLDEADRCTACVTCSRDDLVE |
| | | 1, linker, CD30 domain | KTPCAWNSSRVCECRPGMFCSTSAVNSCARCFFHSVCP |
| | | 2) | AGMIVKFPGTAQKNTVCEPASPGVSPACASPENCKEPSS |
| | | | GTIPQAKPTPVSPATSSASTMPVRGGTRLAQEAASKLTR |
| | | | APDSPSSVGRPSSDPGLSPTQPCPEGSGDCRKQCEPDY |
| | | | YLDEAGRCTACVSCSRDDLVEKTPCAWNSSRTCECRPG |
| | | | MICATSATNSCARCVPYPICAAETVTKPQDMAEKDTTFEA |
| | | | PPLGTQPDCNPTPENGEAPASTSPTQSLLVDSQASKTLPI |
| | | | PTSAPVALSSTGK |
| 224 | Protein | CD30 D1L (CD30 | FPQDRPFEDTCHGNPSHYYDKAVRRCCYRCPMGLFPTQ |
| | | domain 1, linker) | QCPQRPTDCRKQCEPDYYLDEADRCTACVTCSRDDLVE |
| | | | KTPCAWNSSRVCECRPGMFCSTSAVNSCARCFFHSVCP |
| | | | AGMIVKFPGTAQKNTVCEPASPGVSPACASPENCKEPSS |
| | | | GTIPQAKPTPVSPATSSASTMPVRGGTRLAQEAASKLTR |
| | | | APDSPSSVGRPSSDPGLSPTQPCPEGSGDCRKQC |
| 225 | Protein | CD30 LD2 (linker, | PGVSPACASPENCKEPSSGTIPQAKPTPVSPATSSASTM |
| | | CD30 domain 2) | PVRGGTRLAQEAASKLTRAPDSPSSVGRPSSDPGLSPTQ |
| | | | PCPEGSGDCRKQCEPDYYLDEAGRCTACVSCSRDDLVE |
| | | | KTPCAWNSSRTCECRPGMICATSATNSCARCVPYPICAA |
| | | | ETVTKPQDMAEKDTTFEAPPLGTQPDCNPTPENGEAPAS |
| | | | TSPTQSLLVDSQASKTLPIPTSAPVALSSTGK |
| 226 | Protein | CD30 D1D2 (CD30 | FPQDRPFEDTCHGNPSHYYDKAVRRCCYRCPMGLFPTQ |
| | | domain 1, CD30 | QCPQRPTDCRKQCEPDYYLDEADRCTACVTCSRDDLVE |
| | | domain 2) | KTPCAWNSSRVCECRPGMFCSTSAVNSCARCFFHSVCP |
| | | | AGMIVKFPGTAQKNTVCEPASEPDYYLDEAGRCTACVSC |
| | | | SRDDLVEKTPCAWNSSRTCECRPGMICATSATNSCARC |
| | | | VPYPICAAETVTKPQDMAEKDTTFEAPPLGTQPDCNPTP |
| | | | ENGEAPASTSPTQSLLVDSQASKTLPIPTSAPVALSSTGK |

A better understanding of the present disclosure may be obtained through the following examples which are set forth to illustrate, but are not to be construed as limiting, the present disclosure.

### EXAMPLES

### Example 1: Development of Antibody Binding to CD30

### Example 1-1. Selection of Antibody Through Panning

Selection was made of clones binding specifically to CD30 from the Rz library (Korean Patent No. 10-1694832 B, Jan. 12, 2017) by a panning process using the protein CD30-His (Sino Biological, 10777-H08H).

From the Rz library, phage rescue was done using VCSM13 helper phage, and the rescued phages were used in the panning. More than 10¹³ library phages were employed in the first round of binding to the antigen, and a total of five panning rounds was conducted. In the strategy for selectively selecting phages of high affinity, the amount of the antigen is reduced (20 μg, 10 μg, 10 μg, 5 μg, 5 μg), and the number of washes is increased (3, 5, 7, and 10 times) with the increase of the panning round.

Binder phages obtained in each panning round were infected into ER2537 to form colonies which were then examined for binding to the antibody by ELISA. The colonies obtained after infection of the binder phages were inoculated into SB medium (MOPS 10 g/L, Bacto YEAST extract 20 g/L, Trypton 30 g/L) and then grown until reaching an OD600 of 0.8, followed by shaking incubation at 30° C. in the presence of 1 mM IPTG (LPS Solution, IPTG025) to allow the overexpression of the antibody. Periplasmic extraction was conducted using a BBS buffer (200 mM boric acid, 150 mM NaCl, 1 mM EDTA). Binders were screened by ELISA using the extract. For ELISA, the scFv periplasmic extract was applied to a plate coated with 2 μg/mL of CD30-His protein and treated with a secondary antibody (anti-HA-HRP (Roche, 12013819001)). After color development with TMB (BioFx, TMBC-1000-01), OD₄₅₀ values were read using an ELISA reader (Perkin Elmer, Victor3). The ELISA data exhibited affibodies specifically binding to CD30 protein and 11 unique clones were identified by sequencing (Table 1 and Table 2).

TABLE 1
Light chain CDR sequences of
the candidate antibodies

| | 1^{st} | 2^{nd} | 3^{rd} |
|---|---|---|---|
| Anti- | Light Chain | Light Chain | Light Chain |
| body | (LCDR1) | (LCDR2) | (LCDR3) |
| T1-2 | SGSSSNIGSNYVY | RNNQRPS(SEQ | AADSSDDS(SEQ |
| | (SEQ ID NO: 4) | ID NO: 5) | ID NO: 6) |
| T1-7 | SCSSSNIGNNAVS | RNNQRPS(SEQ | AADYGSD(SEQ |
| | (SEQ ID NO: 24) | ID NO: 25) | ID NO: 26) |
| T1-8 | SGSSSNIGSNYVY | RNNQRPS(SEQ | AAYDSYS(SEQ |
| | (SEQ ID NO: 44) | ID NO: 45) | ID NO: 46) |
| T1-9 | SGSSSNIGSNYVY | RNNQRPS(SEQ | AAYYNYN(SEQ |
| | (SEQ ID NO: 64) | ID NO: 65) | ID NO: 66) |
| T1-10 | SGSSSNIGSNYVY | RNNQRPS(SEQ | AADGPYN(SEQ |
| | (SEQ ID NO: 84) | ID NO: 85) | ID NO: 86) |
| T1-23 | SGSSSNIGSNYVY | RNNQRPS(SEQ | AAYRSYD(SEQ |
| | (SEQ ID NO: 104) | ID NO: 105) | ID NO: 106) |
| T1-36 | SGSSSNIGSNYVY | RNNQRPS(SEQ | AAYPSYDS(SEQ |
| | (SEQ ID NO: 124) | ID NO: 125) | ID NO: 126) |
| T1-117 | SGSSSNIGSNYVY | RNNQRPS(SEQ | AADAGNR(SEQ |
| | (SEQ ID NO: 144) | ID NO: 145) | ID NO: 146) |
| T1-141 | SGSSSNIGSNYVY | RNNQRPS(SEQ | AAYSYGY(SEQ |
| | (SEQ ID NO: 164) | ID NO: 165) | ID NO: 166) |
| T1-159 | SGSSSNIGSNYVY | RNNQRPS(SEQ | AAYYPYY(SEQ |
| | (SEQ ID NO: 184) | ID NO: 185) | ID NO: 186) |
| T2-2 | SGSSSNIGSNYVY | RNNQRPS(SEQ | AVSSPYS(SEQ |
| | (SEQ ID NO: 204) | ID NO: 205) | ID NO: 206) |

TABLE 2
Heavy chain CDR sequences of the candidate
antibodies

| | 1^{st} Heavy | 2^{nd} Heavy | 3^{rd} Heavy |
|---|---|---|---|
| Anti- | Chain | Chain | Chain |
| body | (HCDR1) | (HCDR2) | (HCDR3) |
| T1-2 | GYGMS | YISGYSYYTYYADSVKG | YDGNGFDY |
| | (SEQ ID | (SEQ ID NO: 2) | (SEQ ID |
| | NO: 1) | | NO: 3) |
| T1-7 | GYGMS | YISSGSYYTYYADSVKG | YRGDNDYYGYFDY |
| | (SEQ ID | (SEQ ID NO: 22) | (SEQ ID |
| | NO: 21) | | NO: 23) |
| T1-8 | SYMS | SIGSGYYSTYYADSVKG | DYYGGFDY |
| | (SEQ ID | (SEQ ID NO: 42) | (SEQ ID |
| | NO: 41) | | NO: 43) |
| T1-9 | YYGMS | GIGSYSSYTYYADSVKG | YASSPDAYFDY |
| | (SEQ ID | (SEQ ID NO: 62) | (SEQ ID |
| | NO: 61) | | NO: 63) |
| T1-10 | SYGMS | YISGGSYYTYYADSVKG | YGYGYYDGSFDY |
| | (SEQ ID | (SEQ ID NO: 82) | (SEQ ID |
| | NO: 81) | | NO: 83) |
| T1-23 | SYSMS | GIGYPYYTYYADSVKG | YYYDYGFDY |
| | (SEQ ID | (SEQ ID NO: 102) | (SEQ ID |
| | NO: 101) | | NO: 103) |
| T1-36 | YYYMS | YIGGGGSGTYYADSVKG | GPYYGYFDY |
| | (SEQ ID | (SEQ ID NO: 122) | (SEQ ID |
| | NO: 121) | | NO: 123) |
| T1-117 | YYGMS | YISGYSSYTYYADSVKG | YNDSGSFDY |
| | (SEQ ID | (SEQ ID NO: 142) | (SEQ ID |
| | NO: 141) | | NO: 143) |
| T1-141 | YYDMS | GIYGSGSTYYADSVKG | YSYYDSYSDYFDY |
| | (SEQ ID | (SEQ ID NO: 162) | (SEQ ID |
| | NO: 161) | | NO: 163) |
| T1-159 | SYGMS | SIYYYSGGTYYADSVKG | NDYRNNSDFDY |
| | (SEQ ID | (SEQ ID NO: 182) | (SEQ ID |
| | NO: 181) | | NO: 183) |
| T2-2 | SYHMS | GIGYGHGGTYYADSVKG | YDYDYSFDY |
| | (SEQ ID | (SEQ ID NO: 202) | (SEQ ID |
| | NO: 201) | | NO: 203) |

### Example 1-2. Identification of Binding Affinity of Selected Antibody

The eleven selected antibodies and the positive control HRS antibody (Protein Eng Des Sel. 2004 December; 17 (12):847-60.) were cloned in an scFv-Fc-conjugated form (Zb-Fc) and analyzed for binding to CD30 protein and for affinity for CD30-expressing cells.

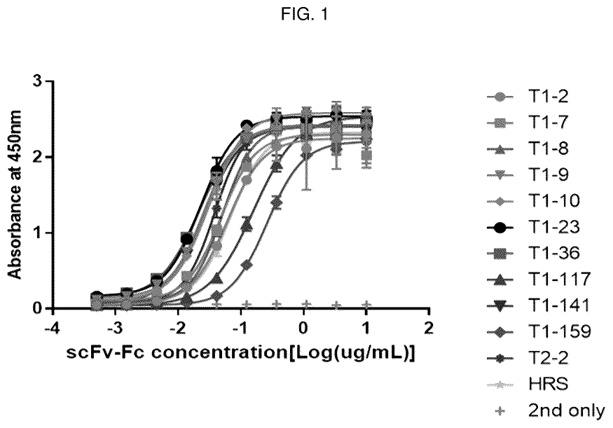

For ELISA, the eleven purified anti-CD30 scFv-Fc forms and the positive control HRS scFV-Fc were 1/3 diluted for 10 points starting from 10 μg/mL in a plate coated with 1 μg/mL CD30-His protein. After treatment with a secondary antibody (anti-hIgG-Fc-HRP (Invitrogen, H10007)), a color was developed with TMB. OD₄₅₀ values were read using an ELISA reader, and EC₅₀ values were measured by means of Graph prism (FIG. 1, Table 3).

As shown in FIG. 1 and Table 3, most of the 11 candidate antibodies had high binding to CD30 compared to the positive control HRS antibody.

TABLE 3
Analyze the binding of candidate antibodies to CD30

| Antibody | EC₅₀(μg/mL) |
|---|---|
| T1-2 | 0.060 |
| T1-7 | 0.048 |
| T1-8 | 0.051 |
| T1-9 | 0.026 |
| T1-10 | 0.031 |
| T1-23 | 0.023 |
| T1-36 | 0.021 |
| T1-117 | 0.157 |
| T1-141 | 0.026 |
| T1-159 | 0.260 |
| T2-2 | 0.038 |
| HRS | 0.067 |

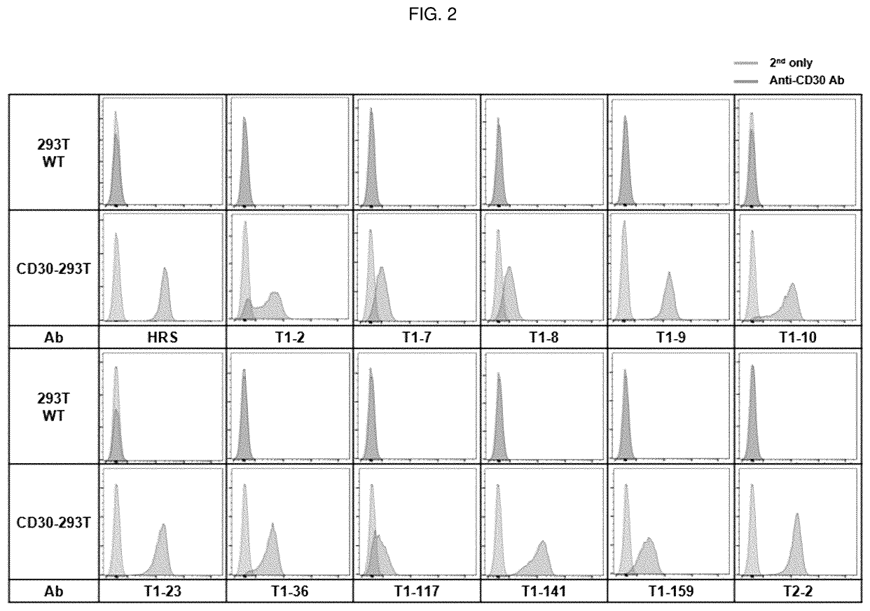

The selected antibodies were analyzed for binding affinity in CD30-expressing cells. For use in this experiment, CD30-293T cell line was established by introducing CD30-overexpressing lentivirus into the 293T cell line that does not express CD30. The CD30-293T cell line and the WT 293T cell line were each prepared at a density of 3×10⁵ cells/tube. The cells were harvested by centrifugation at 1,200 rpm for 3 min and washed with PBS containing 5% FBS. Thereafter, the cells were incubated at 4° C. for 1 hour in the presence of 2 μg/mL of each of the eleven antibodies. The cells were washed three times by three rounds of centrifugation with 200 μL of 5% FBS-containing PBS at 1200 rpm for 3 min. Afterwards, the cells were incubated with 1 μg/mL of anti-human-Fc-FITC (Life Technologies, A11013) at 4° C. for 45 min in a light-shielded condition. After being washed three times through three rounds of centrifugation with 200 μL of 5% FBS-containing PBS at 1200 rpm for 3 min, the cells were measured for fluorescence intensity by FACS (FIG. 2).

As shown in FIG. 2, 11 candidate antibodies were found to bind to the CD30-293T cell line expressing CD30. In particular, T1-9, T1-23, and T2-2 demonstrated binding to CD30-expressing cells at levels equivalent to the positive control, HRS.

### Example 1-3. Identification of CD30-Specific Binding Affinity of Selected Antibody

Examination was made to see whether the selected eleven antibodies bind specifically to CD30. In this regard, the antibodies were measured for binding affinity for the TNFR family members CD27, CD270, CD134, CD137, CD359, and CD30 by ELISA.

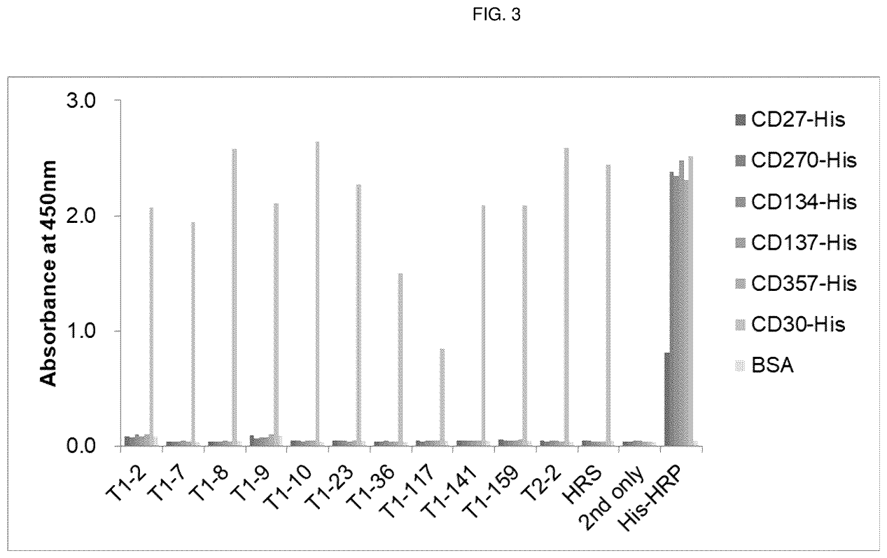

For ELISA, the eleven purified anti-CD30 scFv-Fc forms and the positive control HRS scFV-Fc were each applied at a density of 1 μg/mL to plates coated with TNFR family proteins (CD27-His: Sino Biological, 10039-H08B1; CD134-His: Sino Biological, 10481-H08H; CD137-His: in-house produced; CD270-His: Sino Biological, 10334-H08H; CD357-His: Sino Biological, 13643-H08H; and CD30-His: Sino Biological, 10777-H08H). After treatment with a secondary antibody (anti-hIgG-Fc-HRP (Invitrogen, H10007)), a color was developed with TMB. OD₄₅₀ values were read using an ELISA reader (FIG. 3).

As shown in FIG. 3, all 11 candidate antibodies had high affinity for CD30 only, thus confirming that the candidate antibodies all bind specifically to CD30.

### Example 1-4. Identification of Binding Site of CD30 for Selected Antibody

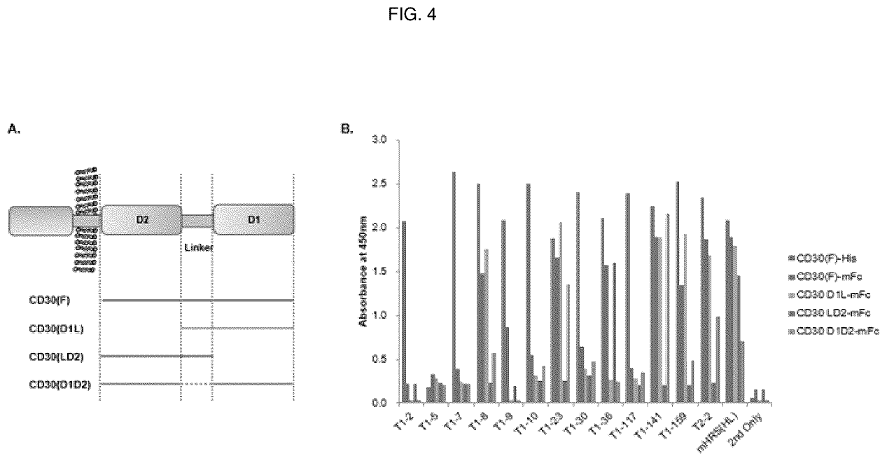

The binding sites of 11 candidate antibodies to the extracellular domain of the CD30 protein were identified by ELISA. To perform the ELISA, the extracellular domain of the CD30 protein was produced in animal cells and used as an antigen. In brief, a DNA structured to link the hinge and Fc region (CH2-CH3) of murine IgG1 to the C-terminus of ECD was cloned into pCEP4 vector (Invitrogen, V044-50) after digestion with the restriction enzymes HindIII and BamHI. Subsequently, the cloned vector was transiently transfected into FreeStyle™ 293F cells (Invitrogen, R790-07) with the aid of polyethylenimine (Polyscience Inc., 23966). From the cell culture, CD30 (F)-mFc, CD30 D1L-mFc, CD30 LD2-mFc, and CD30 D1D2-mFc fusion proteins were purified using Mabselect SuRe resin (Cytiva, 17-5438-01) (FIG. 4A). The purified proteins were quantitated using a protein assay dye (Bio-Rad, 500-0006). Their concentrations and purities were determined by SDS-PAGE with Coomassie blue staining.

The fusion proteins CD30 (F)-mFc, CD30 D1L-mFc, CD30 LD2-mFc, and CD30 D1D2-mFc were each fixed at a concentration of 1 μg/mL overnight, 4° C. to Costar 96-well plates (Corning, 3690). After three washes with TBS-T (0.05% Triton X-100), the protein in each well was blocked at room temperature for one hour with 100 μl of TBS-T/BSA (5% BSA). The blocked plates were washed three times, after which an anti-CD30 antibody was added and incubated at room temperature for one hour to bind to the antigens. The plates were washed three times and then incubated at room temperature for one hour with a 1:3,000 dilution of the secondary antibody anti-human IgG-HRP in TBS-T/BSA to form an antibody conjugate. Three rounds of wash were followed by color development with TMB (SurModics, TMBC-1000-01) at room temperature for 5 min until 1 N sulfuric acid (DukSan, 254) was added to stop the color development. Absorbance at 450 nm was read on Victor X3 (PerkinElmer, 2030-0030) (FIG. 4B).

The positive control HRS antibody was observed to exhibit strong binding strength for CD30 D1L and CD30 LD2 and weak binding strength for CD30 D1D2, indicating that even the linker of CD30 as well as D1 and D2 has influence on the binding. On the other hand, strong binding strength was measured from the T1-36 antibody LD2, T1-8 and T1-159 for D1L, and T1-23 and T1-141 for D1L and D1D2. From this analysis, these antibodies were identified to differ from the positive control HRS antibody in binding site.

### Example 2: Development of Anti-CD30 scFv-Bearing Chimeric Antigen Receptor and Identification of Activity Thereof

### Example 2-1. Construction of Lentivirus Containing Anti-CD30 Antibody Fragment-Linked Chimeric Antigen Receptor

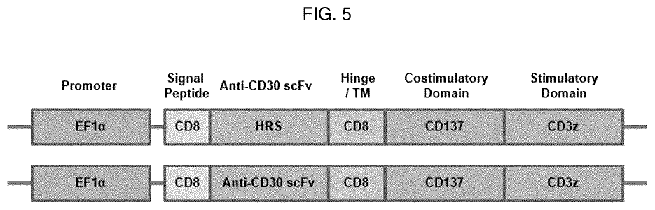

A chimeric antigen receptor was developed using an anti-CD30 antibody fragment. After being subjected to codon optimization for a CD8 leader, a scFv-type anti-CD30, a CD8 hinge and transmembrane region, a CD137 cytoplasmic region, and a CD3 zeta cytoplasmic region in the chimeric antigen receptor, the gene was cut with SpeI/XhoI and ligated to pLenti6-V5/DEST lentiviral vector (Invitrogen, V53306). The constructs thus obtained were identified by base sequencing (FIG. 5).

Each of the prepared lentiviral constructs was transduced, together with the plasmid pCMV-dR8.91 carrying the viral coat protein VSV-G (vesicular stomatitis Indiana virus G protein), gag, pol, and rev genes, into Lenti-X 293T cells (Takara Bio Inc., 632180). Transduction was performed using Lipofectamine 2000 (Invitrogen, 11668019) according to the manufacturer's protocol. After 72 hours, the cell culture containing lentivirus was 10-fold enriched by a centrifugal filter (Millipore, UFC910024) and stored.

### Example 2-2. Preparation of Cytotoxic T Cell Presenting Anti-CD30 Antibody-Bearing Chimeric Antigen Receptor on Surface (CD30-CAR-T)

Cytotoxic T cells on which anti-CD30 antibody fragment-bearing chimeric antigen receptors were presented were prepared using the lentivirus obtained in Example 2-1.

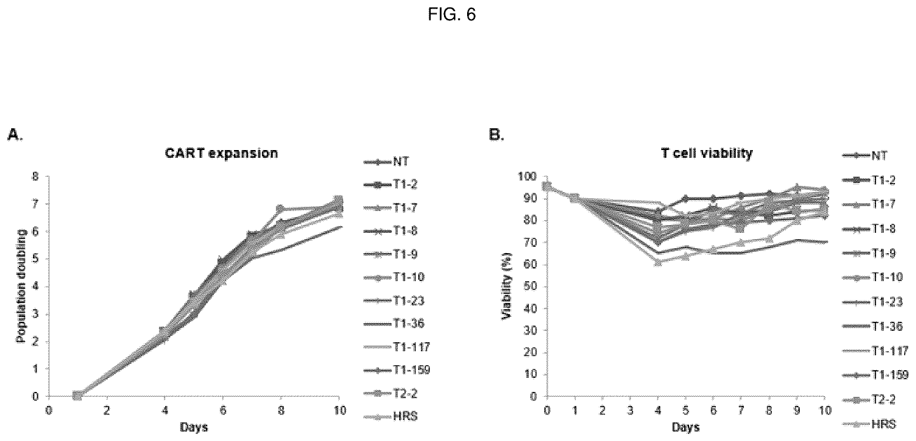

First, human T cells were isolated and stimulated with T cell TransAct (Miltenyi Biotec, 130-111-160) for 24 hours. Thereafter, the lentivirus was transduced for 24 hours into the cells. Then, the medium was exchanged with a medium containing IL-2 (Miltenyi Biotec, 170-076-146), followed by incubation at 37° C. in a 5% CO₂ atmosphere. After being cultured, the CD30-CAR-T was analyzed for proliferation and viability. CD30-CAR-T was prepared, for the most part, with high viability without any specificity in the CAR-T construction process. (FIG. 6, Table 4). The prepared CD30-CAR-Ts were stored in a frozen state at −130° C.

TABLE 4
Analyze proliferation and viability of candidate antibodies

| | Cell viability | Population doubling |
|---|---|---|
| Antibody | (%, Day 10) | (Day 10) |
| T1-2 | 85 | 7.0 |
| T1-7 | 94 | 6.9 |
| T1-8 | 92 | 6.9 |
| T1-9 | 88 | 6.9 |
| T1-10 | 93 | 6.9 |
| T1-23 | 90 | 6.9 |
| T1-36 | 70 | 6.1 |
| T1-117 | 93 | 7.1 |
| T1-159 | 82 | 7.2 |
| T2-2 | 85 | 7.1 |
| HRS | 84 | 6.6 |

Under different conditions, an examination was made again of the proliferative ability of T1-159 observed to exhibit a high cell proliferation rate.

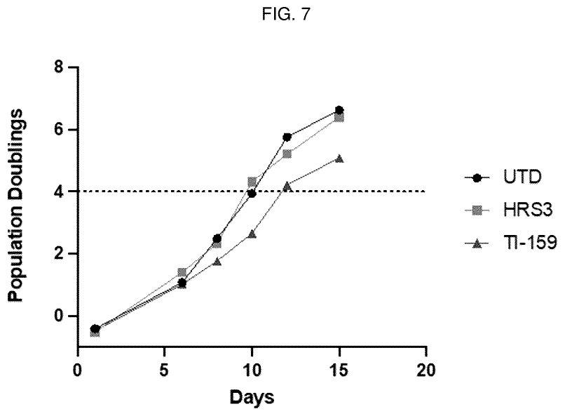

To this end, 2×10⁶ human T cells (CD4:CD8=1:1) were stimulated anti-CD3/CD28 Dynabeads (3:1 bead:T cell) for five days (120 hours). Twenty-four hours after stimulation, lentivirus containing a T1-159 construct was transduced into the T cells (MOI=2). On day 5 (120 hours after stimulation), beads of the CD30-CAR-T cells were removed and a cell concentration of 500,000 T cell/mL was maintained in a cytokine supplemented R10 medium (RPMI1640+10% FBS+20 ng/ml IL7+20 ng/ml IL15). The cell concentration was measured using an OrFlo MoxiGo II benchtop cell counter and a Beckman coulter counter. The results are depicted in FIG. 7. As shown in FIG. 7, the T1-159-containing CAR-T was superb in terms of proliferative rate, proving its proliferative ability sufficient for mass production of CAR-T.

### Example 2-3. Verifying the Manufactured CAR-T Expresses an Anti-CD30 Antibody Fragment

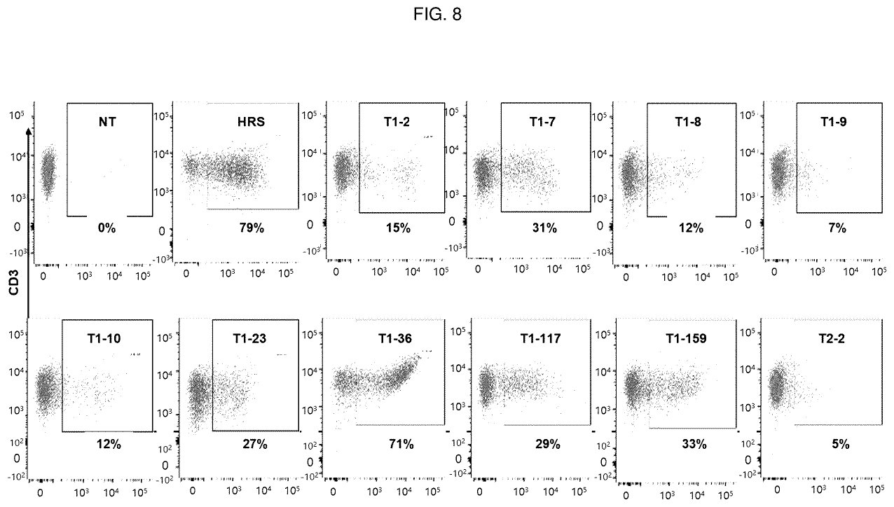

Before evaluating the activity of the CD30-CAR-T, we verified that the manufactured CAR-T was properly expressing the anti-CD30 antibody fragment. Briefly, the CD30-CAR-T cells obtained above were prepared at a density of 3×10⁵ cells/tube, and harvested by centrifugation at 1,200 rpm for 3 min. After a wash with PBS containing 5% FBS, the cells were incubated at 4° C. for 30 min with 2 μg/mL mouse anti-Linker antibody (Clone 163, USA, U.S. Patent No. US 2019/0093101 A, Mar. 28, 2019). The cells were washed three times by three rounds of centrifugation with 200 μL of 5% FBS-containing PBS at 1200 rpm for 3 min. Afterwards, the cells were incubated with 1 μg/mL of anti-mouse-Fc-PE (BioLegend, 405307) at 4° C. for 30 min in a light-shielded condition. After being washed three times through three rounds of centrifugation with 200 μL of 5% FBS-containing PBS at 1200 rpm for 3 min, the expression of CAR was measured in terms of fluorescence intensity by FACS. The results are shown in FIG. 8 and Table 5.

As shown in FIG. 8, the anti-CD30 antibody fragment was expressed in the prepared CAR-T cells, although there were differences in expression levels. In particular, T1-36 had the highest expression levels.

TABLE 5
Expression of the anti-CD30 antibody fragment
in the manufactured CAR-T cells.

| | CAR Expression (%) |
|---|---|
| NT | 0 |
| T1-2 | 15 |
| T1-7 | 31 |
| T1-8 | 12 |
| T1-9 | 7 |
| T1-10 | 12 |
| T1-23 | 27 |
| T1-36 | 71 |
| T1-117 | 29 |
| T1-159 | 33 |
| T2-2 | 5 |
| HRS | 79 |

### Example 3: Identification of Activity of CD30-CAR-T

### Example 3-1. Confirm Cytotoxicity Against CD30+ Cell Lines

To determine whether the CD30-CAR-T prepared in Example 2-2 recognizes CD30 on the cell surface and induces activation of chimeric antigen receptor cells.

The activity of CD30-CAR-T was evaluated by measuring cytotoxicity and IFN release in CD30-positive cells and CD30-CAR-T. Briefly, GFP-Luciferase-expressing lentivirus (Biosettia, GlowCe11-16p-1) was introduced into the CD30-positive cell line SUDHL1 (ATCC, 22268) and CD30-overexpressing CD30-293T cells to establish SUDHL1-ffLuc and CD30-293T-ffLuc cell lines for use in experiments. First, SUDHL1-ffLuc and CD30-293T-ffLuc cells were each seeded at a density of 1×10⁴ cells/well into 96-well plates. The prepared cytotoxic T cells were added at a suitable rate per well to the Luc cells-seeded plate and incubated 37° C. for 24 hours in a 5% CO₂ atmosphere. Thereafter, cytotoxicity of the cytotoxic T cells was measured using a luciferase assay (Bio-Glo Luciferase assay system, Promega, G7941). For measurement of cytotoxic effects, the cytotoxic T cells and the ffLuc cells were co-cultured after which the remaining SUDHL1-ffLuc and CD30-293T-ffLuc cells were lysed with 3× Lysis buffer (75 mM Tris (pH8.0), 30% glycerol, 3% Triton X100) to release a luciferase which was then reacted with a substrate. Relative lysis rates were given when the signal from the wells where only ffLuc cells had been cultured was set forth as 100%.

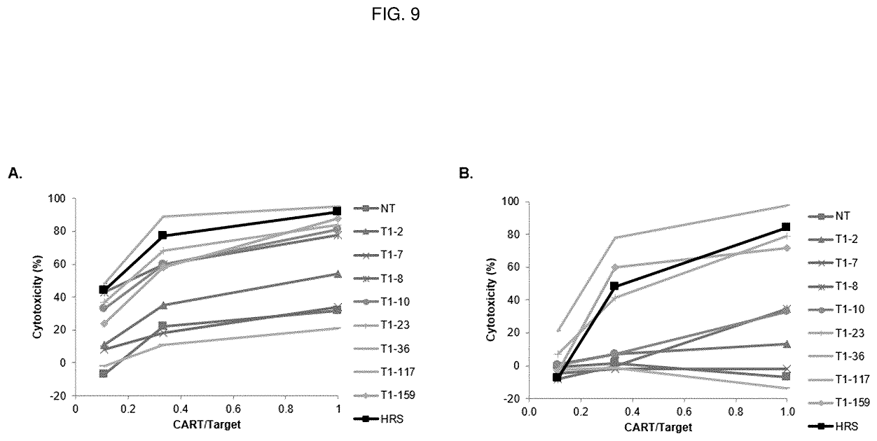

The results are shown in FIG. 9, Table 6, and Table 7. As shown in FIG. 9, the cytotoxic effects were increased in a cytotoxic T cell concentration-dependent manner. Relatively high cytotoxicity was observed in T1-23, T1-36, and T1-159.

TABLE 6
Cytotoxicity (%) of Candidate Antibody against
CD30-Expressing Cell (SUDHL1)

| | CAR-T/Target | | |
|---|---|---|---|
| | 0.11 | 0.33 | 1 |
| NT | −7 | 22 | 32 |
| T1-2 | 11 | 35 | 54 |
| T1-7 | 8 | 18 | 34 |
| T1-8 | 43 | 60 | 78 |
| T1-10 | 33 | 60 | 81 |
| T1-23 | 37 | 68 | 84 |
| T1-36 | 48 | 89 | 95 |
| T1-117 | −2 | 11 | 21 |
| T1-159 | 24 | 58 | 88 |
| HRS | 44 | 77 | 92 |

TABLE 7
Cytotoxicity (%) of Candidate Antibody against
CD30-Expressing Cell (CD30-293T)

| | CAR-T/Target | | |
|---|---|---|---|
| | 0.11 | 0.33 | 1 |
| NT | −1 | 2 | −7 |
| T1-2 | 1 | 7 | 13 |
| T1-7 | −5 | −2 | −2 |
| T1-8 | −8 | 0 | 35 |
| T1-10 | 1 | 7 | 33 |
| T1-23 | 7 | 41 | 79 |
| T1-36 | 21 | 78 | 98 |
| T1-117 | −3 | −1 | −14 |
| T1-159 | −4 | 60 | 72 |
| HRS | −8 | 48 | 84 |

T1-36 and T1-159 that exhibited excellent cytotoxicity effects were reexamined for cytotoxicity against HDLM-2 cells (CD30+ cHL cell line).

HDLM-2 cells (CD30+ cHL) were seeded, together with 50,000 CBG (click-beetle green), into 96-well plates, with the target and effector cells set at various ratios. UTD, HRS3, and T1-36 or T1-159 CD30-CAR-T cells of the same donor (ND578) were treated. After 72 hours, 20 μL of luciferin (GoldBio, diluted to 150 μg/mL in PBS) was added to each well and incubated for 10 minutes in an incubator (37° C.+5% CO₂). Then, luminance was acquired using BioTek Synergy H4, and a tumor death effect in each well was evaluated according to Equation 1, below.

$$Tumor Death ( \% ) = ( maximal luminance ( cancer alone ) - coculture well ) / ( maximal luminance ( cancer alone ) - background signal ) * 100 \qquad [ Equation 1 ]$$

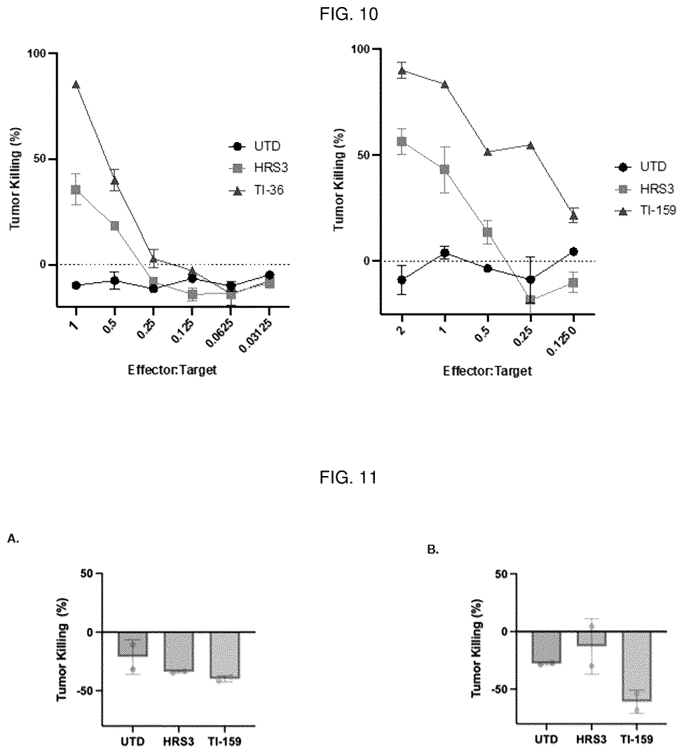

Results of the assay for tumor death effects are depicted in FIG. 10.

As shown in FIG. 10, CD30-CAR-T containing T1-36 and T1-159 were both observed to exhibit excellent tumor death effects. In particularly, T1-159 exhibited a tumor death rate of 50% or higher even when the ratio of CAR-T to the target was 0.25, proving its relatively high ability to kill tumors even at a low concentration.

### Example 3-2. Assay for Cytotoxicity Against CD30-Cell Line

T1-159, observed to have excellent cytotoxic effect, was assayed for an off-target effect by examining it has cytotoxicity against the CD30-cell line.

OCI-Ly18 cells (CD19+ DLBCL) or Nalm6 cells (CD19+ B-ALL) were seeded, together with 50,000 CBG (click-beetle green), into 96-well plates, with the target and effector cells set at various ratios. UTD, HRS3, and T1-159 CD30-CAR-T cells of the same donor (ND578) were treated. After 48 hours, 20 μL of luciferin (GoldBio, diluted to 150 μg/mL in PBS) was added to each well and incubated for 10 minutes in an incubator (37° C.+5% CO₂). Then, luminance was acquired using BioTek Synergy H4, and a tumor death effect in each well was evaluated according to Equation 1, above.

Results are depicted in FIG. 11. As shown in FIG. 11, the T1-159-containing CD30-CAR-T did not cytotoxicity against OCI-Ly18 and Nalm6 cells, which did not express CD30, indicating that T1-159 is very specific for CD30, with a low plausibility of off-target effect.

### Example 3-3. Validate the Effectiveness of Stimulating Interferon-Gamma Release

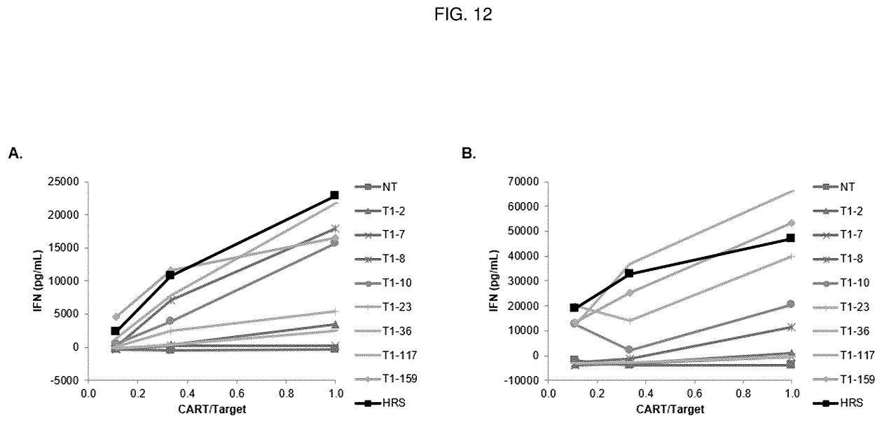

As for IFN release, the IFN secreted to culture media of the CD30-expressing cells and the CD30-CAR-T were quantitated using an ELISA kit (Human IFN-gamma ELISA Set, BD biosciences, 555142) according to the manufacturer's protocol (FIG. 12). As can be seen in FIG. 12, IFN release increased with the increasing of the treated cytotoxic T cell ratio. Consistent with the data of cytotoxicity, IFN release was increased in T1-23, T1-36, and T1-159.

### Example 4: Assay for Anti-Tumoral Effect of T1-159-Containing CD30-CAR-T (In Vivo)

Eight NSG mice (8 weeks old) were subcutaneously implanted at the right flank with 1×10⁶ HDLM-2 cells. After 55 days, the tumors had amounted to significant sizes (150-200 mm³). Mice were randomly assigned to two groups of four. One group was injected with 300,000 HRS3-containing CD30-CAR-T cells and the other group was injected with 300,000 T1-159-containing CD30-CAR-T cells. Tumor size was monitored weekly via a digital caliper. Tumor volumes were calculated using Equation 2 below.

$$Tumor volume ( {mm}^{3} ) = 0.5 \times L \times {( W )}^{2} \qquad Equation 2$$

wherein, L=length or long axis of tumor (mm); W=width or short axis of tumor (mm).

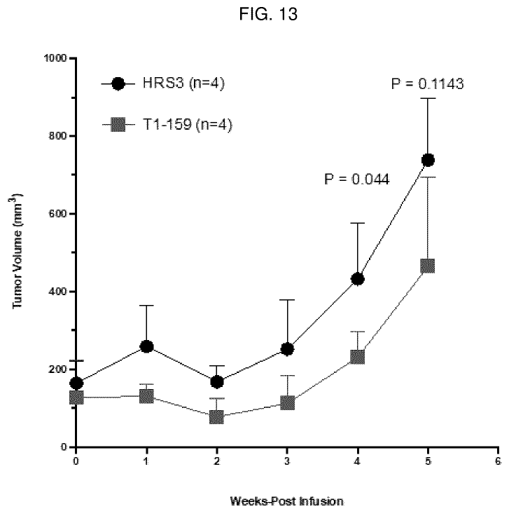

Results are depicted in FIG. 13. As shown in FIG. 13, CD30-CAR-T containing T1-159 had a significantly lower rate of tumor volume increase over the entire period than CD30-CAR-T containing HRS3. From 1 week after CAR-T treatment, the group treated with the CD30-CAR-T containing HRS3 showed a 50% larger tumor volume than the group treated with the CD30-CAR-T containing T1-159. This data indicates that the CD30-CAR-T containing T1-159 exhibits a very good antitumoral effect.

What is claimed is:

1. An anti-CD30 antibody or an antigen binding fragment thereof, comprising an immunoglobulin heavy-chain variable region domain and an immunoglobulin light-chain variable region domain, wherein:
i) the heavy-chain variable region domain comprises HCDR1, HCDR2, and HCDR3, wherein the HCDR1, HCDR2, and HCDR3 comprise the amino acid sequence of SEQ ID NO: 1, 2, and 3, respectively; and the light-chain variable region domain comprise LCDR1, LCDR2, and LCDR3, wherein the LCDR1, LCDR2, and LCDR3 comprise the amino acid sequence of SEQ ID NO: 4, 5, and 6, respectively;
ii) the heavy-chain variable region domain comprises HCDR1, HCDR2, and HCDR3, wherein the HCDR1, HCDR2, and HCDR3 comprise the amino acid sequence of SEQ ID NO: 21, 22, and 23, respectively; and the light-chain variable region domain comprises LCDR1, LCDR2, and LCDR3, wherein the LCDR1, LCDR2, and LCDR3 comprise the amino acid sequence of SEQ ID NO: 24, 25, and 26, respectively;
iii) the heavy-chain variable region domain comprises HCDR1, HCDR2, and HCDR3, wherein the HCDR1, HCDR2, and HCDR3 comprise the amino acid sequence of SEQ ID NO: 41, 42, and 43, respectively; and the light-chain variable region domain comprises LCDR1, LCDR2, and LCDR3, wherein the LCDR1, LCDR2, and LCDR3 comprise the amino acid sequence of SEQ ID NO: 44, 45, and 46, respectively;
iv) the heavy-chain variable region domain comprises HCDR1, HCDR2, and HCDR3, wherein the HCDR1, HCDR2, and HCDR3 comprise the amino acid sequence of SEQ ID NO: 61, 62, and 63, respectively; and the light-chain variable region domain comprise LCDR1, LCDR2, and LCDR3, wherein the LCDR1, LCDR2, and LCDR3 comprises the amino acid sequence of SEQ ID NO: 64, 65, and 66, respectively;
v) the heavy-chain variable region domain comprises HCDR1, HCDR2, and HCDR3, wherein the HCDR1, HCDR2, and HCDR3 comprise the amino acid sequence of SEQ ID NO: 81, 82, and 83, respectively; and the light-chain variable region domain comprise LCDR1, LCDR2, and LCDR3, wherein the LCDR1, LCDR2, and LCDR3 comprises the amino acid sequence of SEQ ID NO: 84, 85, and 86, respectively;
vi) the heavy-chain variable region domain comprises HCDR1, HCDR2, and HCDR3, wherein the HCDR1, HCDR2, and HCDR3 comprise the amino acid sequence of SEQ ID NO: 101, 102, and 103, respectively; and the light-chain variable region domain comprises LCDR1, LCDR2, and LCDR3, wherein the LCDR1, LCDR2, and LCDR3 comprise the amino acid sequence of SEQ ID NO: 104, 105, and 106, respectively;
vii) the heavy-chain variable region domain comprises HCDR1, HCDR2, and HCDR3, wherein the HCDR1, HCDR2, and HCDR3 comprise the amino acid sequence of SEQ ID NO: 121, 122, and 123, respectively; and the light-chain variable region domain comprise LCDR1, LCDR2, and LCDR3, wherein the LCDR1, LCDR2, and LCDR3 comprise the amino acid sequence of SEQ ID NO: 124, 125, and 126, respectively;
viii) the heavy-chain variable region domain comprises HCDR1, HCDR2, and HCDR3, wherein the HCDR1, HCDR2, and HCDR3 comprise the amino acid sequence of SEQ ID NO: 141, 142, and 143, respectively; and the light-chain variable region domain comprises LCDR1, LCDR2, and LCDR3, wherein the LCDR1, LCDR2, and LCDR3 comprise the amino acid sequence of SEQ ID NO: 144, 145, and 146, respectively;
ix) the heavy-chain variable region domain comprises HCDR1, HCDR2, and HCDR3, wherein the HCDR1, HCDR2, and HCDR3 comprise the amino acid sequence of SEQ ID NO: 161, 162, and 163, respectively; and the light-chain variable region domain comprises LCDR1, LCDR2, and LCDR3, wherein the LCDR1, LCDR2, and LCDR3 comprise the amino acid sequence of SEQ ID NO: 164, 165, and 166, respectively;
x) the heavy-chain variable region domain comprises HCDR1, HCDR2, and HCDR3, wherein the HCDR1, HCDR2, and HCDR3 comprise the amino acid sequence of SEQ ID NO: 181, 182, and 183, respectively; and the light-chain variable region domain comprise LCDR1, LCDR2, and LCDR3, wherein the LCDR1, LCDR2, and LCDR3 comprise the amino acid sequence of SEQ ID NO: 184, 185, and 186, respectively; or
xi) the heavy-chain variable region domain comprises HCDR1, HCDR2, and HCDR3, wherein the HCDR1, HCDR2, and HCDR3 comprise the amino acid sequence of SEQ ID NO: 201, 202, and 203, respectively; and the light-chain variable region domain comprise LCDR1, LCDR2, and LCDR3, wherein the LCDR1, LCDR2, and LCDR3 comprise the amino acid sequence of SEQ ID NO: 204, 205, and 206, respectively.

2. The anti-CD30 antibody or the antigen binding fragment thereof according to claim 1, wherein the anti-CD30 antibody or the antigen binding fragment thereof comprises a heavy chain variable region domain and a light chain variable region domain, wherein:
i) the heavy-chain variable region domain comprises the amino acid sequence of SEQ ID NO: 7; and the light-chain variable region domain comprises the amino acid sequence of SEQ ID NO: 8;
ii) the heavy-chain variable region domain comprises the amino acid sequence of SEQ ID NO: 27; and the light-chain variable region domain comprises the amino acid sequence of SEQ ID NO: 28;
iii) the heavy-chain variable region domain comprises the amino acid sequence of SEQ ID NO: 47; and the light-chain variable region domain comprises the amino acid sequence of SEQ ID NO: 48;
iv) the heavy-chain variable region domain comprises the amino acid sequence of SEQ ID NO: 67; and the light-chain variable region domain comprises the amino acid sequence of SEQ ID NO: 68;
v) the heavy-chain variable region domain comprises the amino acid sequence of SEQ ID NO: 87; and the light-chain variable region domain comprises the amino acid sequence of SEQ ID NO: 88;
vi) the heavy-chain variable region domain comprises the amino acid sequence of SEQ ID NO: 107; and the light-chain variable region domain comprises the amino acid sequence of SEQ ID NO: 108;
vii) the heavy-chain variable region domain comprises the amino acid sequence of SEQ ID NO: 127; and the light-chain variable region domain comprises the amino acid sequence of SEQ ID NO: 128;
viii) the heavy-chain variable region domain comprises the amino acid sequence of SEQ ID NO: 147; and the light-chain variable region domain comprises the amino acid sequence of SEQ ID NO: 148;
ix) the heavy-chain variable region domain comprises the amino acid sequence of SEQ ID NO: 167; and the light-chain variable region domain comprises the amino acid sequence of SEQ ID NO: 168;
x) the heavy-chain variable region domain comprises the amino acid sequence of SEQ ID NO: 187; and the light-chain variable region domain comprises the amino acid sequence of SEQ ID NO: 188; or
xi) the heavy-chain variable region domain comprises the amino acid sequence of SEQ ID NO: 207; and the light-chain variable region domain comprises the amino acid sequence of SEQ ID NO: 208.

3. The anti-CD30 antibody or the antigen binding fragment thereof according to claim 1, wherein the anti-CD30 antibody or the antigen binding fragment thereof comprises scFv, wherein the scFv comprises an amino acid sequence selected from the group consisting of the amino acid sequence of SEQ ID NO: 9, the amino acid sequence of SEQ ID NO: 29, the amino acid sequence of SEQ ID NO: 49, the amino acid sequence of SEQ ID NO: 69, the amino acid sequence of SEQ ID NO: 89, the amino acid sequence of SEQ ID NO: 109, the amino acid sequence of SEQ ID NO: 129, the amino acid sequence of SEQ ID NO: 149, the amino acid sequence of SEQ ID NO: 169, the amino acid sequence of SEQ ID NO: 189, and the amino acid sequence of SEQ ID NO: 209.

4. The anti-CD30 antibody or the antigen binding fragment thereof according to claim 1, wherein the antigen binding fragment is Fab, Fab′, F(ab′)₂, Fv, scFV, or chemically linked F(ab′)₂.

5. A nucleic acid molecular encoding the anti-CD30 antibody or the antigen binding fragment thereof according to claim 1.

6. The nucleic acid molecule according to claim 5, wherein the nucleic acid molecule comprises nucleotide sequences selected from the group consisting of:
i) the nucleotide sequence of SEQ ID NO: 11, the nucleotide sequence of SEQ ID NO: 12, the nucleotide sequence of SEQ ID NO: 13, the nucleotide sequence of SEQ ID NO: 14, the nucleotide sequence of SEQ ID NO: 15, and the nucleotide sequence of SEQ ID NO: 16, wherein the nucleotide sequence encodes HCDR1, HCDR2, HCDR3, LCDR1, LCDR2, and LCDR3, respectively;
ii) the nucleotide sequence of SEQ ID NO: 31, the nucleotide sequence of SEQ ID NO: 32, the nucleotide sequence of SEQ ID NO: 33, the nucleotide sequence of SEQ ID NO: 34, the nucleotide sequence of SEQ ID NO: 35, and the nucleotide sequence of SEQ ID NO: 36, wherein the nucleotide sequence encodes HCDR1, HCDR2, HCDR3, LCDR1, LCDR2, and LCDR3, respectively;
iii) the nucleotide sequence of SEQ ID NO: 51, the nucleotide sequence of SEQ ID NO: 52, the nucleotide sequence of SEQ ID NO: 53, the nucleotide sequence of SEQ ID NO: 54, the nucleotide sequence of SEQ ID NO: 55, and the nucleotide sequence of SEQ ID NO: 56, wherein the nucleotide sequence encodes HCDR1, HCDR2, HCDR3, LCDR1, LCDR2, and LCDR3, respectively;
iv) the nucleotide sequence of SEQ ID NO: 71, the nucleotide sequence of SEQ ID NO: 72, the nucleotide sequence of SEQ ID NO: 73, the nucleotide sequence of SEQ ID NO: 74, the nucleotide sequence of SEQ ID NO: 75, and the nucleotide sequence of SEQ ID NO: 76, wherein the nucleotide sequence encodes HCDR1, HCDR2, HCDR3, LCDR1, LCDR2, and LCDR3, respectively;
v) the nucleotide sequence of SEQ ID NO: 91, the nucleotide sequence of SEQ ID NO: 92, the nucleotide sequence of SEQ ID NO: 93, the nucleotide sequence of SEQ ID NO: 94, the nucleotide sequence of SEQ ID NO: 95, and the nucleotide sequence of SEQ ID NO: 96, wherein the nucleotide sequence encodes HCDR1, HCDR2, HCDR3, LCDR1, LCDR2, and LCDR3, respectively;
vi) the nucleotide sequence of SEQ ID NO: 111, the nucleotide sequence of SEQ ID NO: 112, the nucleotide sequence of SEQ ID NO: 113, the nucleotide sequence of SEQ ID NO: 114, the nucleotide sequence of SEQ ID NO: 115, and the nucleotide sequence of SEQ ID NO: 116, wherein the nucleotide sequence encodes HCDR1, HCDR2, HCDR3, LCDR1, LCDR2, and LCDR3, respectively;
vii) the nucleotide sequence of SEQ ID NO: 131, the nucleotide sequence of SEQ ID NO: 132, the nucleotide sequence of SEQ ID NO: 133, the nucleotide sequence of SEQ ID NO: 134, the nucleotide sequence of SEQ ID NO: 135, and the nucleotide sequence of SEQ ID NO: 136, wherein the nucleotide sequence encodes HCDR1, HCDR2, HCDR3, LCDR1, LCDR2, and LCDR3, respectively;
viii) the nucleotide sequence of SEQ ID NO: 151, the nucleotide sequence of SEQ ID NO: 152, the nucleotide sequence of SEQ ID NO: 153, the nucleotide sequence of SEQ ID NO: 154, the nucleotide sequence of SEQ ID NO: 155, and the nucleotide sequence of SEQ ID NO: 156, wherein the nucleotide sequence encodes HCDR1, HCDR2, HCDR3, LCDR1, LCDR2, and LCDR3, respectively;
ix) the nucleotide sequence of SEQ ID NO: 171, the nucleotide sequence of SEQ ID NO: 172, the nucleotide sequence of SEQ ID NO: 173, the nucleotide sequence of SEQ ID NO: 174, the nucleotide sequence of SEQ ID NO: 175, and the nucleotide sequence of SEQ ID NO: 176, wherein the nucleotide sequence encodes HCDR1, HCDR2, HCDR3, LCDR1, LCDR2, and LCDR3, respectively;
x) the nucleotide sequence of SEQ ID NO: 191, the nucleotide sequence of SEQ ID NO: 192, the nucleotide sequence of SEQ ID NO: 193, the nucleotide sequence of SEQ ID NO: 194, the nucleotide sequence of SEQ ID NO: 195, and the nucleotide sequence of SEQ ID NO: 196, wherein the nucleotide sequence encodes HCDR1, HCDR2, HCDR3, LCDR1, LCDR2, and LCDR3, respectively; and
xi) the nucleotide sequence of SEQ ID NO: 211, the nucleotide sequence of SEQ ID NO: 212, the nucleotide sequence of SEQ ID NO: 213, the nucleotide sequence of SEQ ID NO: 214, the nucleotide sequence of SEQ ID NO: 215, and the nucleotide sequence of SEQ ID NO: 216, wherein the nucleotide sequence encodes HCDR1, HCDR2, HCDR3, LCDR1, LCDR2, and LCDR3, respectively.

7. The nucleic acid molecule according to claim 5, wherein the nucleic acid molecule comprises nucleotide sequences selected from the group consisting of:
i) the nucleotide sequence of SEQ ID NO: 17 and the nucleotide sequence of SEQ ID NO: 18, wherein the nucleotide sequence encodes heavy chain variable domain and light chain variable domain, respectively;
ii) the nucleotide sequence of SEQ ID NO: 37 and the nucleotide sequence of SEQ ID NO: 38, wherein the nucleotide sequence encodes heavy chain variable domain and light chain variable domain, respectively;
iii) the nucleotide sequence of SEQ ID NO: 57 and the nucleotide sequence of SEQ ID NO: 58, wherein the nucleotide sequence encodes heavy chain variable domain and light chain variable domain, respectively;
iv) the nucleotide sequence of SEQ ID NO: 77 and the nucleotide sequence of SEQ ID NO: 78, wherein the nucleotide sequence encodes heavy chain variable domain and light chain variable domain, respectively;
v) the nucleotide sequence of SEQ ID NO: 97 and the nucleotide sequence of SEQ ID NO: 98, wherein the nucleotide sequence encodes heavy chain variable domain and light chain variable domain, respectively;
vi) the nucleotide sequence of SEQ ID NO: 117 and the nucleotide sequence of SEQ ID NO: 118, wherein the nucleotide sequence encodes heavy chain variable domain and light chain variable domain, respectively;
vii) the nucleotide sequence of SEQ ID NO: 137 and the nucleotide sequence of SEQ ID NO: 138, wherein the nucleotide sequence encodes heavy chain variable domain and light chain variable domain, respectively;
viii) the nucleotide sequence of SEQ ID NO: 157 and the nucleotide sequence of SEQ ID NO: 158, wherein the nucleotide sequence encodes heavy chain variable domain and light chain variable domain, respectively;
ix) the nucleotide sequence of SEQ ID NO: 177 and the nucleotide sequence of SEQ ID NO: 178, wherein the nucleotide sequence encodes heavy chain variable domain and light chain variable domain, respectively;
x) the nucleotide sequence of SEQ ID NO: 197 and the nucleotide sequence of SEQ ID NO: 198, wherein the nucleotide sequence encodes heavy chain variable domain and light chain variable domain, respectively; and
xi) the nucleotide sequence of SEQ ID NO: 217 and the nucleotide sequence of SEQ ID NO: 218, wherein the nucleotide sequence encodes heavy chain variable domain and light chain variable domain, respectively.

8. The nucleic acid molecule according to claim 5, wherein the nucleic acid molecule comprises a nucleotide sequence selected from the group consisting of the nucleotide sequence of SEQ ID NO: 19, the nucleotide sequence of SEQ ID NO: 39, the nucleotide sequence of SEQ ID NO: 59, the nucleotide sequence of SEQ ID NO: 79, the nucleotide sequence of SEQ ID NO: 99, the nucleotide sequence of SEQ ID NO: 119, the nucleotide sequence of SEQ ID NO: 139, the nucleotide sequence of SEQ ID NO: 159, the nucleotide sequence of SEQ ID NO: 179, the nucleotide sequence of SEQ ID NO: 199, and the nucleotide sequence of SEQ ID NO: 219, wherein the nucleotide sequence encodes scFv.

9. A CD30-specific chimeric antigen receptor, comprising:
an anti-CD30 antibody or an antigen binding fragment thereof;
a transmembrane domain; and
an intracellular signaling domain,
wherein the anti-CD30 antibody or the antigen binding fragment thereof comprises a heavy chain variable region domain and a light chain variable region domain, wherein:
i) the heavy-chain variable region domain comprises HCDR1, HCDR2, and HCDR3, wherein the HCDR1, HCDR2, and HCDR3 comprise the amino acid sequence of SEQ ID NO: 1, 2, and 3, respectively; and the light-chain variable region domain comprises LCDR1, LCDR2, and LCDR3, wherein the LCDR1, LCDR2, and LCDR3 comprise the amino acid sequence of SEQ ID NO: 4, 5, and 6, respectively;
ii) the heavy-chain variable region domain comprises HCDR1, HCDR2, and HCDR3, wherein the HCDR1, HCDR2, and HCDR3 comprise the amino acid sequence of SEQ ID NO: 21, 22, and 23, respectively; and the light-chain variable region domain comprises LCDR1, LCDR2, and LCDR3, wherein the LCDR1, LCDR2, and LCDR3 comprise the amino acid sequence of SEQ ID NO: 24, 25, and 26, respectively;
iii) the heavy-chain variable region domain comprises HCDR1, HCDR2, and HCDR3, wherein the HCDR1, HCDR2, and HCDR3 comprise the amino acid sequence of SEQ ID NO: 41, 42, and 43, respectively; and the light-chain variable region domain comprises LCDR1, LCDR2, and LCDR3, wherein the LCDR1, LCDR2, and LCDR3 comprise the amino acid sequence of SEQ ID NO: 44, 45, and 46, respectively;
iv) the heavy-chain variable region domain comprises HCDR1, HCDR2, and HCDR3, wherein the HCDR1, HCDR2, and HCDR3 comprise the amino acid sequence of SEQ ID NO: 61, 62, and 63, respectively; and the light-chain variable region domain comprises LCDR1, LCDR2, and LCDR3, wherein the LCDR1, LCDR2, and LCDR3 comprise the amino acid sequence of SEQ ID NO: 64, 65, and 66, respectively;
v) the heavy-chain variable region domain comprises HCDR1, HCDR2, and HCDR3, wherein the HCDR1, HCDR2, and HCDR3 comprise the amino acid sequence of SEQ ID NO: 81, 82, and 83, respectively; and the light-chain variable region domain comprises LCDR1, LCDR2, and LCDR3, wherein the LCDR1, LCDR2, and LCDR3 comprise the amino acid sequence of SEQ ID NO: 84, 85, and 86, respectively;
vi) the heavy-chain variable region domain comprises HCDR1, HCDR2, and HCDR3, wherein the HCDR1, HCDR2, and HCDR3 comprise the amino acid sequence of SEQ ID NO: 101, 102, and 103, respectively; and the light-chain variable region domain comprises LCDR1, LCDR2, and LCDR3, wherein the LCDR1, LCDR2, and LCDR3 comprise the amino acid sequence of SEQ ID NO: 104, 105, and 106, respectively;
vii) the heavy-chain variable region domain comprises HCDR1, HCDR2, and HCDR3, wherein the HCDR1, HCDR2, and HCDR3 comprise the amino acid sequence of SEQ ID NO: 121, 122, and 123, respectively; and the light-chain variable region domain comprises LCDR1, LCDR2, and LCDR3, wherein the LCDR1, LCDR2, and LCDR3 comprise the amino acid sequence of SEQ ID NO: 124, 125, and 126, respectively;
viii) the heavy-chain variable region domain comprises HCDR1, HCDR2, and HCDR3, wherein the HCDR1, HCDR2, and HCDR3 comprise the amino acid sequence of SEQ ID NO: 141, 142, and 143, respectively; and the light-chain variable region domain comprises LCDR1, LCDR2, and LCDR3, wherein the LCDR1, LCDR2, and LCDR3 comprise the amino acid sequence of SEQ ID NO: 144, 145, and 146, respectively;
ix) the heavy-chain variable region domain comprises HCDR1, HCDR2, and HCDR3, wherein the HCDR1, HCDR2, and HCDR3 comprise the amino acid sequence of SEQ ID NO: 161, 162, and 163, respectively; and the light-chain variable region domain comprises LCDR1, LCDR2, and LCDR3, wherein the LCDR1, LCDR2, and LCDR3 comprise the amino acid sequence of SEQ ID NO: 164, 165, and 166, respectively;
x) the heavy-chain variable region domain comprises HCDR1, HCDR2, and HCDR3, wherein the HCDR1, HCDR2, and HCDR3 comprise the amino acid sequence of SEQ ID NO: 181, 182, and 183, respectively; and the light-chain variable region domain comprises LCDR1, LCDR2, and LCDR3, wherein the LCDR1, LCDR2, and LCDR3 comprise the amino acid sequence of SEQ ID NO: 184, 185, and 186, respectively; or
xi) the heavy-chain variable region domain comprises HCDR1, HCDR2, and HCDR3, wherein the HCDR1, HCDR2, and HCDR3 comprise the amino acid sequence of SEQ ID NO: 201, 202, and 203, respectively; and the light-chain variable region domain comprises LCDR1, LCDR2, and LCDR3, wherein the LCDR1, LCDR2, and LCDR3 comprise the amino acid sequence of SEQ ID NO: 204, 205, and 206, respectively.

10. The CD30-specific chimeric antigen receptor of claim 9, wherein the anti-CD30 antibody or the antigen binding fragment thereof comprises a heavy chain variable region domain and a light chain variable region domain, wherein:
i) the heavy-chain variable region domain comprises the amino acid sequence of SEQ ID NO: 7; and the light-chain variable region domain comprises the amino acid sequence of SEQ ID NO: 8;
ii) the heavy-chain variable region domain comprises the amino acid sequence of SEQ ID NO: 27; and the light-chain variable region domain comprises the amino acid sequence of SEQ ID NO: 28;
iii) the heavy-chain variable region domain comprises the amino acid sequence of SEQ ID NO: 47; and the light-chain variable region domain comprises the amino acid sequence of SEQ ID NO: 48;
iv) the heavy-chain variable region domain comprises the amino acid sequence of SEQ ID NO: 67; and the light-chain variable region domain comprises the amino acid sequence of SEQ ID NO: 68;
v) the heavy-chain variable region domain comprises the amino acid sequence of SEQ ID NO: 87; and the light-chain variable region domain comprises the amino acid sequence of SEQ ID NO: 88;
vi) the heavy-chain variable region domain comprises the amino acid sequence of SEQ ID NO: 107; and the light-chain variable region domain comprises the amino acid sequence of SEQ ID NO: 108;
vii) the heavy-chain variable region domain comprises the amino acid sequence of SEQ ID NO: 127; and the light-chain variable region domain comprises the amino acid sequence of SEQ ID NO: 128;
viii) the heavy-chain variable region domain comprises the amino acid sequence of SEQ ID NO: 147; and the light-chain variable region domain comprises the amino acid sequence of SEQ ID NO: 148;
ix) the heavy-chain variable region domain comprises the amino acid sequence of SEQ ID NO: 167; and the light-chain variable region domain comprises the amino acid sequence of SEQ ID NO: 168;
x) the heavy-chain variable region domain comprises the amino acid sequence of SEQ ID NO: 187; and the light-chain variable region domain comprises the amino acid sequence of SEQ ID NO: 188; or
xi) the heavy-chain variable region domain comprises the amino acid sequence of SEQ ID NO: 207; and the light-chain variable region domain comprises the amino acid sequence of SEQ ID NO: 208.

11. The CD30-specific chimeric antigen receptor of claim 9, wherein the anti-CD30 antibody or the antigen binding fragment thereof comprises scFv, wherein the scFv comprises an amino acid sequence selected from the group consisting of the amino acid sequence of SEQ ID NO: 9, the amino acid sequence of SEQ ID NO: 29, the amino acid sequence of SEQ ID NO: 49, the amino acid sequence of SEQ ID NO: 69, the amino acid sequence of SEQ ID NO: 89, the amino acid sequence of SEQ ID NO: 109, the amino acid sequence of SEQ ID NO: 129, the amino acid sequence of SEQ ID NO: 149, the amino acid sequence of SEQ ID NO: 169, the amino acid sequence of SEQ ID NO: 189, and the amino acid sequence of SEQ ID NO: 209.

12. The CD30-specific chimeric antigen receptor of claim 9, wherein the CD30-specific chimeric antigen receptor comprises scFv CAR, wherein the scFc CAR comprises an amino acid sequence selected from the group consisting of the amino acid sequence of SEQ ID NO:10, the amino acid sequence of SEQ ID NO: 30, the amino acid sequence of SEQ ID NO: 50, the amino acid sequence of SEQ ID NO: 70, the amino acid sequence of SEQ ID NO: 90, the amino acid sequence of SEQ ID NO: 110, the amino acid sequence of SEQ ID NO: 130, the amino acid sequence of SEQ ID NO: 150, the amino acid sequence of SEQ ID NO: 170, the amino acid sequence of SEQ ID NO: 190, and the amino acid sequence of SEQ ID NO: 210.

13. The CD30-specific chimeric antigen receptor of claim 9, wherein the transmembrane domain is a protein selected from the consisting of T-cell receptor, CD27, CD28, CD3 epsilon, CD45, CD4, CD5, CD8, CD9, CD16, CD22, CD33, CD37, CD64, CD80, CD86, CD134, CD137, and CD154.

14. The CD30-specific chimeric antigen receptor of claim 9, wherein the signaling domain comprises a functional signaling domain from at least one of 4-1BB, CD28, OX40, and CD3 zeta.

15. A nucleic acid molecule encoding the CD30-specific chimeric antigen receptor of claim 9.

16. An effector cell expressing the CD30-specific chimeric antigen receptor of claim 9.

17. A pharmaceutical composition comprising the anti-CD30 antibody or antigen-binding fragment of claim 1.

18. A method for treating a disease related with a CD30-expressing cell, the method comprising:
a step of administering the pharmaceutical composition of claim 17 to a subject in need thereof.
